(19) United States
(12) Patent Application Publication
(10) Pub. No.: US 2025/0277045 A1
(43) Pub. Date: Sep. 4, 2025

(54) DEVELOPMENT AND APPLICATION OF T-CELL ENGAGER THERAPEUTIC AGENT

(71) Applicant: KEYMED BIOSCIENCES CO.,LTD, Chengdu (CN)

(72) Inventors: Bo CHEN, Chengdu (CN); Gang XU, Chengdu (CN); Ying WANG, Chengdu (CN)

(21) Appl. No.: 18/265,117

(22) PCT Filed: Dec. 2, 2021
(86) PCT No.: PCT/CN2021/135121

§ 371 Date: Jun. 2, 2023

(30) Foreign Application Priority Data
Dec. 2, 2020 (CN) 202011402180.8

(51) Int. Cl. C07K 16/28; A61K 39/00; A61P 35/00

(52) U.S. Cl. CPC C07K 16/2878; A61P 35/00; C07K 16/2809; A61K 2039/505; C07K 2317/24; C07K 2317/31; C07K 2317/92

(57) ABSTRACT

The present disclosure relates to the development and application of a T-cell engager therapeutic agent. A T-cell engager is a T-cell bispecific antibody, and the bispecific antibody contains a first binding domain bound to BCMA on the surface of a target cell and a second binding domain bound to CD3 on the surface of a T cell. The bispecific antibody has good tolerance, can effectively block the binding of the BCMA to a receptor APRIL, and has no adverse reaction, such as weight loss.

### CROSS REFERENCE TO THE RELATED APPLICATIONS

This application is a National Stage application under 35 U.S.C. § 371 of International Application No. PCT/CN2021/135121, filed on Dec. 2, 2021, which is based upon and claims priority to Chinese Patent Application No. 202011402180.8, filed on Dec. 2, 2020, the entire contents of which are incorporated herein by reference.

### SEQUENCE LISTING

This application contains a Sequence Listing that has been submitted electronically as an ASCII text file named “51426-0007US1_UPDATEDSL_ST25.txt.” The ASCII text file, created on Aug. 21, 2024, is 149,177 bytes in size. The material in the ASCII text file is hereby incorporated by reference in its entirety.

### TECHNICAL FIELD

The present disclosure relates to the development and application of a T-cell engager therapeutic agent, particularly a bispecific antibody bound to BCMA and CD3, and applications thereof.

### BACKGROUND ART

B-cell maturation antigen (BCMA), as a member of the tumor necrosis factor receptor superfamily, is widely expressed on the surface of myeloma cells, only expressed on plasma cells in normal tissues, but not on naive B cells, CD34+ hematopoietic stem cells, and other normal tissues. BCMA-deficient mice have normal B cell development and humoral immune response. In multiple myeloma patient samples, after the ligand APRIL (A Proliferation Inducing Ligand) binds to BCMA, it may stimulate myeloma cell growth and proliferation, and prevent tumor cell apoptosis simultaneously. Since the expression of BCMA in malignant plasma cells is significantly higher than that in normal plasma cells, BCMA has become an important target for immunotherapy.

In hematological malignancies, multiple myeloma is the second most common disease, frequently occurring in the elderly. Accumulation of malignantly proliferating plasma cells in the patient's bone marrow may lead to bone marrow failure and loss of immune function, and is often accompanied by multiple osteolytic lesions, hypercalcemia, anemia, and renal damage simultaneously. Although new drugs, including proteasome inhibitors, immunomodulators, and targeted antibodies, have significantly prolonged patient survival in recent years, nearly all patients relapse. New therapies that are being developed such as CAR-T cells (chimeric antigen receptor T-cells) have a complicated preparation process, high treatment cost, and high recurrence rate; antibody drug conjugates (ADC) are limited to low doses due to the safety of conjugated toxins; although BCMA bispecific antibodies previously developed are clinically shown to be quite effective, increased doses are accompanied with increased side effects, which may lead to a strong cytokine storm syndrome and endanger patient life. Thus, there remains a need in the art to develop new, safer, and more effective drugs.

A T-cell bispecific antibody (or referred to as T-cell engager) is a specially constructed antibody molecule that recognizes a target cell surface antigen (antigen arm) at one end and binds to a T-cell CD3 receptor (CD3 arm) at the other end. Using antibodies that target the ε chain of CD3, the CD3 on T cells is aggregated in a manner like TCR/peptide/HLA, thereby activating T cells and killing tumors. The BCMA×CD3 bispecific molecule is a T-cell TCB antibody that targets BCMA expressed on myeloma cells and the CD3ε chain (CD3e) present on T cells. The action mechanism of the BCMA×CD3 bispecific molecule involves binding to both BCMA+ myeloma cells and CD3+ T cells, resulting in T cell activation and T cell-mediated cell killing.

During a normal immune response, TCR binds with low affinity (about 1-100 μM) to an exogenous peptide-human leukocyte antigen complex (HLLA) on infected or mutated cells, transduces an activation signal into the nucleus via CD3, activates the expression of transcription factors and their downstream proteins (cytokines, granzymes, perforin, etc.), wherein the signal intensity produced by the TCR complex will determine the fate of T cells. The ε, γ, δ, and ζ subunits of the signaling complex associate with each other to form CD3ε-γ heterodimers, CD3ε-δ heterodimers, and CD3ζ-ζ homodimers. The early developed CD3 bispecific antibodies were mostly based on a few murine antibodies such as OKT3, L2K, UCHT1, and TR66, and have high affinity. In clinical studies, it was found that excessively strong affinity of CD3 antibody would lead to excessive activation of T cells and release of a large number of cytokines, forming cytokine storm syndrome; at the same time, high affinity also leads to the enrichment of bispecific antibodies in secondary lymphoid organs, reducing the exposure to tumor tissue. The binding ability of the Fc portion of the antibody to the Fcγ receptor is another important factor affecting drug safety. Since the Fcγ receptor is expressed in various normal tissues, after bispecific antibody binds to the Fcγ receptor on cell membrane through Fc, it may result in cross-linking activation of CD3 receptor bound at the other end due to Fcγ receptor aggregation, thus resulting in severe off-target toxicity, for example early marketed catumaxomab triggered rapid cytokine release due to Fc fragment binding to Fcy receptors expressed by liver Kupffer cells. By using a human IgG2 subtype or IgG4 subtype which has a weak Fc γ receptor binding ability, or further performing amino acid substitutions at the corresponding position in CH2, for example, Armour et al. substituted positions 233-236 (EU sequence numbering) of IgG1 and IgG4 with the corresponding sequence of IgG2, reduced binding to Fc γ receptors (Armour K L et al., Recombinant human IgG molecules lacking Fc gamma receptor I binding and monocyte triggering activities, Eur J Immunol. 1999 August; 29(8):2613-24), Newman et al. introduced Ser228Pro and Leu235Glu mutations in IgG4 to stabilize the IgG4 structure while reducing binding to Fc γ receptors (Newman R. et al., Modification of the Fc region of a primatized IgG antibody to human CD4 retains its ability to modulate CD4 receptors but does not deplete CD4(+) T cells in chimpanzees, Clin Immunol. 2001 February; 98(2):164-74), Idusogie et al. found that alanine substitution at positions Asp270, Lys322, Pro329, or Pro331 significantly reduced the binding ability of IgG1 to complement component C1q (Idusogie E E et al., Mapping of the C1q Binding Site on Rituxan, a Chimeric Antibody with a Human IgG1 Fc, J Immunol Apr. 15, 2000, 164 (8) 4178-4184), etc.

### SUMMARY OF THE INVENTION

In order to solve the problems in the prior art, the present disclosure provides a novel BCMA antibody having a high affinity to the BCMA protein. Also, the present disclosure provides novel BCMA-CD3 κλ bispecific antibodies formed by BCMA antibodies and CD3 antibodies. By adopting a full-length IgG configuration, introducing charge, and optimizing affinity, BCMA-CD3 κλ bispecific antibody allows the κλ bispecific antibody preferentially to localize in BCMA+ tumor tissues, can recruit activated T cells at a low concentration, and thus causes effective killing to target cells, but does not activate T cells in the absence of target cells. At the same time, κλ bispecific antibodies do not bind to receptors such as FcγRI, FcγRIIA, FcγRIIIA, etc. thereby reducing the risk of cytokine storm occurring. It is proved in a mouse model with reconstituted human immune system that, this novel BCMA-CD3 κλ bispecific antibody can effectively inhibit tumor growth, and the efficacy was superior to the same class of antibodies.

In one aspect, the present disclosure provides a BCMA-binding antibody or antigen-binding portion thereof.

In one aspect, the present disclosure provides a bispecific antibody or antigen-binding portion thereof.

In one aspect, the present disclosure provides a nucleic acid encoding bispecific antibodies or antigen-binding portions thereof as previously described.

In one aspect, the present disclosure provides a vector comprising a nucleic acid as previously described.

In one aspect, the present disclosure provides a cell comprising a nucleic acid or vector as previously described.

The antibody or antigen-binding portion thereof according to the foregoing aspects, wherein the antibody or antigen-binding portion thereof as previously described is humanized.

In one aspect, the present disclosure provides a pharmaceutical composition or kit comprising an antibody or antigen-binding portion thereof, or nucleic acid encoding the same as previously described, and a pharmaceutically acceptable carrier.

In one aspect, the present disclosure provides antibody-drug conjugates comprising the foregoing antibodies or antigen-binding portions thereof as previously described, bispecific or multispecific molecules covalently attached to a therapeutic moiety.

In one aspect, the present disclosure provides a method of treating a disorder associated with BCMA, comprising the steps of administering to a mammal a therapeutically effective amount of an antibody or antigen-binding fragment thereof, nucleic acid, vector, cell, and/or pharmaceutical composition as previously described.

In one aspect, the present disclosure provides a use of the foregoing antibodies or antigen-binding fragments thereof, nucleic acids, vectors, cells, and/or pharmaceutical compositions in the manufacture of a drug or kit for treating a BCMA-associated disorder in a mammal.

The antibodies of the present disclosure can be used in a variety of applications, including the detection of BCMA proteins, diagnosis, treatment, or prevention of diseases associated with BCMA.

### BRIEF DESCRIPTION OF THE DRAWINGS

FIG. 1 shows SDS-PAGE results of human, cynomolgus monkey BCMA-mFc bivalent recombinant protein and BCMA-hFc kih monovalent recombinant protein.

FIG. 2 shows that, both human and cynomolgus monkey BCMA stably transfected cells are detected by flow cytometry, both human BCMA stably transfected cells (CHO-hBCMA-2C2) and cynomolgus monkey BCMA stably transfected cells (CHO-cynoBCMA-1A2) express high levels of BCMA.

FIG. 3 shows SDS-PAGE results for the CD3εγ-Fc recombinant protein.

FIG. 4 shows binding of humanized CD3 antibody to CD3εγ-Fc recombinant protein.

FIG. 5 shows binding of humanized CD3 antibodies to Jurkat cells.

FIG. 6 shows binding of BCMA×CD3 κλ bispecific antibodies to BCMA stably transfected cells.

FIG. 7 shows high-affinity binding of BCMA×CD3 κλ bispecific antibodies to BCMA+ tumor cells.

FIG. 8 shows binding of BCMA×CD3 κλ bispecific antibodies to Jurkat cells.

FIG. 9 shows binding of BCMA×CD3 κλ bispecific antibodies to human peripheral blood T cells.

FIG. 10 shows killing of NCI-H929 cells and activation of T cells by the BCMA×CD3 κλ bispecific antibody, wherein FIG. 10A shows killing of NCI-H929 cells, and FIG. 10B shows activation of T cells.

FIG. 11 shows killing of RPMI-8226 cells and activation of T cells by the BCMA×CD3 κλ bispecific antibody, wherein FIG. 11A shows killing of RPMI-8226 cells, and FIG. 11B shows activation of T cells.

FIG. 12 shows activation of the T cell signaling pathway by the BCMA×CD3 κλ bispecific antibody.

FIG. 13 shows activation of peripheral blood T cells by the BCMA×CD3 κλ bispecific antibody.

FIG. 14 shows binding of BCMA×CD3 κλ bispecific antibodies to activated FcγR receptors.

FIG. 15 shows blocking of BCMA binding to the receptor APRIL by a BCMA×CD3 κλ bispecific antibody.

FIG. 16 shows inhibitory effect of BCMA×CD3 κλ bispecific antibodies on the subcutaneous NCI-H929 xenograft tumor model in immunodeficient mice.

### DETAILED DESCRIPTION OF EMBODIMENTS

### I. Definitions

In the present disclosure, the scientific and technical terms used herein have the meanings commonly understood by those skilled in the art, unless otherwise specified. Also, as used herein, protein and nucleic acid chemistry, molecular biology, cell and tissue culture, microbiology, immunology, and laboratory procedures used herein are terms and routine procedures widely used in the corresponding fields. Meanwhile, to better understand the present disclosure, definitions and explanations of related terms are provided below.

As used herein, the term “BCMA” may refer to the concept of BCMA itself and any variant, isotype, and paralog thereof, which are present together in animals and preferably in humans.

The term “human BCMA” refers to BCMA of human origin and may preferably have, but is not limited to, the amino acid sequence of Genbank accession number AB052772.1.

The terms “anti-BCMA antibody” and “BCMA-binding antibody” refer to an antibody that is capable of binding to BCMA with sufficient affinity such that the antibody is useful as a diagnostic and/or therapeutic agent in targeting BCMA. In one embodiment, the degree of binding of an anti-BCMA antibody to an unrelated non-BCMA protein is less than about 10% of the binding of the antibody to BCMA, as measured, for example, by a radioimmunoassay (RIA). In certain embodiments, a BCMA-binding antibody has a dissociation constant (K_{d}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M). In certain embodiments, anti-BCMA antibodies bind BCMA epitopes conserved among BCMA from different species.

“CD3” refers to any native CD3 from any vertebrate source, including mammals, such as primates (e.g. humans), non-human primates (e.g. cynomolgus monkey), and rodents (e.g. mice and rats), unless otherwise specified. The term encompasses “full-length” unprocessed CD3 as well as any form of CD3 derived from processing in cells. The term also encompasses naturally occurring variants of CD3, such as splicing variants or allelic variants. In one embodiment, the CD3 is human CD3, in particular, the a subunit (CD3ε) of human CD3. The amino acid sequence of human CD3ε is shown under UniProt (www.uniprot.org) accession number P07766 (version 144), or NCBI (www.ncbi.nlm.nih.gov/) RefSeq NP_000724.1. The amino acid sequence of cynomolgus monkey CD3ε is shown under NCBI GenBank no. BAB71849.1.

The term “cell surface” is used in accordance with its normal meaning in the art and thus includes cell exterior accessible by binding to proteins and other molecules.

As used herein, unless otherwise specified, the term “about” or “approximately” means within plus or minus 10% of a given value or range. Where an integer is required, the term refers to an integer rounding up or down to the nearest integer within plus or minus 10% of the given value or range.

The phrase “substantially identical” with respect to polypeptide chain sequences of an antibody is understood to mean an antibody that exhibits at least 60%, 65%, 70%, 75%, 80%, 85%, 90%, 95%, 96%, 97%, 98%, 99% or more sequence identity to a reference polypeptide chain sequence. With respect to a nucleic acid sequence, the term is understood to mean a nucleotide sequence that exhibits at least greater than 60%, 65%, 70%, 75%, 80%, 85%, 90%, 95%, 96%, 97%, 98%, 99% or more sequence identity to a reference nucleic acid sequence.

Sequence “similarity” or “identity” has well-known meanings in the field, and the percentage of sequence identity between two nucleic acid or polypeptide molecules or regions can be calculated using published techniques. Sequence identity can be measured along the full-length polynucleotide or polypeptide, or along a region of the molecule. Although there are many methods of measuring the identity between two polynucleotides or polypeptides, the term “identity” is well known to technicians (Carrillo, H. & Lipman, D. SIAM J Applied Math 48: 1073 (1988)).

A “substituted” variant is a variant in which at least one amino acid residue in the native sequence has been removed and a different amino acid is inserted at its same position. The substitution may be single, wherein only one amino acid in the molecule is substituted; or maybe multiple amino acids are substituted, wherein two or more amino acids are substituted in the same molecule. Multiple substitutions may be at consecutive sites. Likewise, an amino acid may be substituted with multiple residues, wherein such variants include both substitutions and insertions. An “insertion” variant is a variant in which one or more amino acids are inserted immediately adjacent to an amino acid at a particular position in a native sequence. The immediately adjacent amino acids is meant attached to the α-carboxyl or α-amine functional groups of the amino acid. A “deletion” variant is a variant in which one or more amino acids in the native amino acid sequence have been removed. Typically, deletion variants have one or two amino acids deleted in a particular region of the molecule.

With respect to the variable domains of antibodies, the term “variable” refers to certain portions of related molecules that differ widely in sequence among antibodies and are used for specific recognition and binding of a particular antibody to its specific target. However, the variability is not uniformly distributed throughout the variable domain of the antibody. The variability is concentrated in what is known as the complementarity determining regions (CDRs; i.e. CDR1, CDR2, and CDR3) or three segments of the hypervariable region, all of which are located within the variable domains of the light chains and heavy chains. The highly conserved portions within the variable domains are referred to as framework (FR) regions or framework sequences. Each variable domain of a native heavy chain and light chain comprises four FR regions, which predominantly adopt a β-folded configuration, they are linked by three CDRs, the CDRs form loops, the loops are linked to the β-folded configuration and in some cases the loops form part of the β-folded configuration. The CDRs of each chain are typically linked together in close proximity by FR regions and are helpful for the formation of target binding sites (epitopes or determinants) of antibodies by use of CDR from other chains. As used herein, the numbering of amino acid residue of immunoglobulin is performed in accordance with the amino acid residue numbering system of immunoglobulin edited by Kabat et al., unless otherwise indicated. One CDR may have the ability to specifically bind to an associated epitope.

As used herein, an “antibody fragment” or “antigen-binding fragment” of an antibody refers to any portion of a full-length antibody that is less than full-length, but comprises at least a portion of the variable region (e.g. one or more CDR and/or one or more antibody binding sites) of the antibody that binds antigen, and thus the “antibody fragment” or “antigen-binding fragment” retains binding specificity as well as at least a portion of the specific binding capacity of the full-length antibody. Therefore, an antigen-binding fragment refers to an antibody fragment that comprises an antigen-binding portion that binds to the same antigen as the antibody from which the antibody fragment is derived. Antibody fragments include antibody derivatives produced by enzymatic treatment of full-length antibodies, as well as synthetically produced derivatives, e.g. recombinantly produced derivatives. Antibodies include antibody fragments. Examples of antibody fragments include, but are not limited to, Fab, Fab′, F(ab′)₂, single chain Fv (scFv), Fv, dsFv, diabodies, Fd and Fd′ fragments, and other fragments, including modified fragments. The fragments may comprise multiple chains linked together, for example by disulfide bonds and/or by peptide linkers. Antibody fragments generally comprise at least or about 50 amino acids, and typically at least or about 200 amino acids. An antigen-binding fragment includes any antibody fragment that, when the antibody fragment is inserted into an antibody framework (e.g. by displacement of the corresponding region), it results in an antibody that immunospecifically binds (i.e. exhibits a Ka of at least or at least about 10⁷-10⁹M⁻¹) to an antigen. A “functional fragment” or “analog of an anti-BCMA antibody” is a fragment or analog that prevents or substantially reduces the binding ability of the receptor to a ligand or initiate signal transduction. As used herein, functional fragments generally have the same meaning as “antibody fragments”, and in the case of antibodies, fragments that prevent or substantially reduce the binding ability of the receptor to a ligand or initiate signal transduction, e.g. Fv, Fab, F(ab′)₂, and the like. A “Fv” fragment consists of a dimer (V_{H}-V_{L} dimer) formed by a variable domain of a heavy chain and a variable domain of a light chain in a non-covalent association. In this configuration, the three CDRs of each variable domain interact with each other to determine a target binding site on the surface of the V_{H}-V_{L} dimer, as is the case with intact antibodies. The six CDRs jointly confer target binding specificity to the intact antibody. However, even a single variable domain (or half of an Fv comprising only three target-specific CDRs) may still have the ability to recognize and bind targets.

As used herein, the term “bispecific antibody (BsAb)” refers to antibodies and/or antigen-binding molecules capable of specifically binding to two different antigene determinants. Generally, a bispecific antibody and/or antigen-binding molecule comprises two antigen-binding sites, each of which is specific for a different antigene determinant. In certain embodiments, the bispecific antibody and/or antigen-binding molecule is capable of binding two antigene determinants simultaneously, particularly two antigene determinants expressed on two different cells.

As used herein, “monoclonal antibody” refers to a population of identical antibodies, meaning that each individual antibody molecule in the population of monoclonal antibodies is identical to another antibody molecule. This property is in contrast to the property of a polyclonal population of antibodies, the polyclonal population of antibodies comprises antibodies having a plurality of different sequences. Monoclonal antibodies can be prepared by many well-known methods (Smith et al. (2004) J. Clin. Pathol. 57, 912-917; and Nelson et al., J Clin Pathol (2000), 53, 111-117). For example, monoclonal antibodies can be made by immortalizing B cells, for example, by fusion with myeloma cells to produce hybridoma cell lines, or by infecting B cells with a virus such as EBV. Recombinant techniques can also be used to prepare antibodies from clonal populations of host cells in vitro by transforming host cells with plasmids carrying artificial sequences encoding the nucleotides of the antibodies.

As used herein, the term “hybridoma” or “hybridoma cell” refers to a cell or cell line (typically a myeloma or lymphoma cell) resulting from the fusion of antibody-producing lymphocytes and non-antibody-producing cancer cells. As is known to those skilled in the art, hybridomas can be propagated and continuously supplied to produce a particular monoclonal antibody. Methods for producing hybridomas are known in the art. When referring to the term “hybridoma” or “hybridoma cell”, it also includes subclones and progeny cells of hybridomas.

As used herein, a full-length antibody is an antibody having two full-length heavy chains (e.g. VH-CH1-CH2-CH3 or VH-CH1-CH2-CH3-CH4), two full-length light chains (VL-CL) and a hinge region, e.g. an antibody naturally produced by an antibody-secreting B cell as well as a synthetically produced antibody having the same domain.

The term “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

A “humanized” antibody refers to a form of non-human (e.g. mouse) antibody that is a chimeric immunoglobulin, immunoglobulin chain or fragment thereof (e.g. Fv, Fab, Fab′, F(ab′)₂ or other antigen-binding subsequences of an antibody) that contains a minimal sequence derived from non-human immunoglobulin. Preferably, the humanized antibody is a human immunoglobulin (recipient antibody) in which residues of the complementarity-determining region (CDR) of the recipient antibody are replaced by CDR residues from a non-human species (donor antibody) such as mouse, rat, or rabbit having the desired specificity, affinity, and capacity.

Furthermore, in humanization it is also possible to mutate amino acid residues within the CDR1, CDR2, and/or CDR3 regions of VH and/or VL, thereby improving one or more binding properties (e.g. affinity) of the antibody. Mutations can be introduced, e.g. by PCR-mediated mutagenesis, and their effect on antibody binding or other functional properties can be assessed using the in vitro or in vivo assays described herein. Typically, conservative mutations are introduced. Such mutations may be amino acid substitutions, additions, or deletions. In addition, mutations within CDR typically do not exceed one or two. Thus, the humanized antibodies of the present disclosure also encompass antibodies comprising one or two amino acid mutations within CDR.

As used herein, the term “CDR” refers to a complementarity-determining region, as is known that, each heavy chain and light chain of an antibody molecule has three CDRs. CDR is also known as a hypervariable region and is present in the variable region of each of the heavy chain and light chains of an antibody, and has sites with very high variability in the primary structure of CDR. In the present specification, the heavy chain CDR of is represented by CDR1, CDR2, and CDR3 from the amino terminus of the amino-terminal sequence of the heavy chain, and the light chain CDR is represented by CDR1, CDR2, CDR3 from the amino terminus of the amino-terminal sequence of the light chain. These sites are adjacent to each other in the tertiary structure and determine the specificity of the antigen to which the antibody binds.

As used herein, the term “epitope” refers to any antigene determinant on an antigen bound to the paratope of an antibody. Epitope determinants usually comprise chemically active surface types of molecules, such as amino acids or sugar side chains, and usually have specific three-dimensional structural characteristics as well as specific charge characteristics.

As used herein, “specific binding” or “immunospecifically binding” with respect to an antibody or antigen-binding fragment thereof is used interchangeably herein and refers to the ability of an antibody or antigen-binding fragment to form one or more non-covalent bonds with the same antigen through non-covalent interactions between the antibody and the antibody binding site of the antigen. The antigen may be an isolated antigen or present in a tumor cell. Generally, an antibody that immunospecifically binds (or specifically binds) to an antigen binds the antigen with an affinity constant Ka of about or 1×10⁷M⁻¹ or 1×10⁸M⁻¹ or more (or a dissociation constant (Kd) of 1×10⁻⁷M or 1×10⁻⁸M or less). Affinity constants can be determined by standard kinetic methods for antibody reactions, e.g. immunoassays, surface plasmon resonance (SPR) (Rich and Myszka (2000) Curr. Opin. Biotechnol 11:54; Englebienne (1998) Analyst. 123:1599), isothermal titration calorimetry (ITC), or other kinetic interaction assays known in the art; see also U.S. Pat. No. 7,229,619 which describes an exemplary SPR and ITC method for calculating the binding affinity of an antibody). Instruments and methods for detecting and monitoring the rate of binding in real-time are known and commercially available (see, BiaCore 2000, Biacore AB, Upsala, Sweden and GE Healthcare Life Sciences; Malmqvist (2000) Biochem. Soc. Trans. 27:335).

As used herein, the terms “polynucleotide” and “nucleic acid molecule” refer to an oligomer or polymer comprising at least two linked nucleotides or nucleotide derivatives, including deoxyribonucleic acid (DNA) and ribonucleic acid (RNA), typically linked together by phosphodiester bonds. As used herein, the term “nucleic acid molecule” is intended to include DNA molecules and RNA molecules. The nucleic acid molecule may be single-stranded or double-stranded and may be cDNA.

As used herein, an isolated nucleic acid molecule is a nucleic acid molecule isolated from other nucleic acid molecules present in the natural source of the nucleic acid molecule. An “isolated” nucleic acid molecule, such as a cDNA molecule, can be substantially free of other cellular material or culture medium when prepared by recombinant techniques, or substantially free of chemical precursors or other chemical ingredients when chemically synthesized. Exemplary isolated nucleic acid molecules provided herein include isolated nucleic acid molecules encoding a provided antibody or antigen-binding fragment.

As used herein, “operably linked” with respect to a nucleic acid sequence, region, element or domain means that the nucleic acid regions are functionally related to one another. For example, a promoter may be operably linked to a nucleic acid encoding a polypeptide such that the promoter regulates or mediates transcription of the nucleic acid.

Also provided are “conservative sequence modifications” with respect to the sequences set forth in the sequence listings described herein, i.e. nucleotide sequence and amino acid sequence modifications that do not eliminate binding of an antibody encoded by a nucleic acid or containing an amino acid sequence to an antigen. These conservative sequence modifications include conservative nucleotide substitutions and amino acid substitutions, and nucleotide additions and amino acid additions, and nucleotide deletions and amino acid deletions. For example, modifications can be introduced into the sequence listings described herein by standard techniques known in the art, such as site-directed mutagenesis and PCR-mediated mutagenesis. Conservative sequence modifications include conservative amino acid substitutions, wherein an amino acid residue is replaced with an amino acid residue having a similar side chain. Families of amino acid residues having similar side chains have been defined in the art. These families include amino acids with basic side chains (e.g. lysine, arginine, histidine), amino acids with acidic side chains (e.g. aspartic acid, glutamic acid), amino acids with uncharged polar side chains (e.g. glycine, asparagine, glutamine, serine, threonine, tyrosine, cysteine, tryptophan), amino acids with nonpolar side chains (e.g. alanine, valine, leucine, isoleucine, proline, phenylalanine, methionine), amino acids with β-branched side chains (e.g. threonine, valine, isoleucine) and amino acids with aromatic side chains (e.g. tyrosine, phenylalanine, tryptophan, histidine). Thus, a predicted nonessential amino acid residue in an anti-BCMA antibody is preferably replaced with another amino acid residue from the same side chain family. Methods for identifying conservative substitutions of nucleotides and amino acids that do not eliminate antigen binding are well known in the art (For example, see Brummell et al., Biochem. 32: 1180-1187 (1993); Kobayashi et al., Protein Eng. 12(10): 879-884 (1999); Burks et al., Proc. Natl. Acad. Sci. USA 94: 412-417 (1997)).

Alternatively, in another embodiment, mutations can be randomly introduced along all or a portion of the anti-BCMA antibody coding sequence, e.g. by saturation mutagenesis, and the resulting modified anti-BCMA antibodies can be screened for improved binding activity.

As used herein, “expression” refers to a process of producing a polypeptide by transcription and translation of a polynucleotide. The expression level of a polypeptide can be assessed using any method known in the art, including, for example, methods of determining the amount of polypeptide produced from a host cell. Such methods may include, but are not limited to, quantitation of polypeptides in cell lysates by ELISA, gel electrophoresis followed by Coomassie blue staining, Lowry protein assay, and Bradford protein assay.

As used herein, a “host cell” is a cell for receiving, maintaining, replicating, and amplifying a vector. Host cells may also be used to express a polypeptide encoded by the vector. As the host cell divides, the nucleic acid contained in the vector replicates, thereby amplifying the nucleic acids. The host cell may be a eukaryotic cell or a prokaryotic cell. Suitable host cells include, but are not limited to, CHO cells, various COS cells, HeLa cells, and HEK cells such as HEK 293 cells.

As used herein, a “vector” is a replicable nucleic acid from which one or more heterologous proteins can be expressed when the vector is transformed into an appropriate host cell. With respect to vectors, they include vectors into which a nucleic acid encoding a polypeptide or fragment thereof may be introduced, typically by restriction enzyme digestion and ligation, with respect to vectors, they also include vectors comprising a nucleic acid encoding a polypeptide. Vectors are used to introduce a nucleic acid encoding a polypeptide into a host cell, to amplify the nucleic acid, or to express/display the polypeptide encoded by the nucleic acid. The vectors usually remain episomal, but can be designed such that the gene or part thereof is integrated into the chromosome of the genome. Also contemplated are vectors for artificial chromosomes, such as yeast artificial chromosome vectors and mammalian artificial chromosomes. The selection and use of such vehicles are well-known to those skilled in the art.

As used herein, a vector also includes a “virus vector” or a “viral vector”. A viral vector is an engineered virus operably linked to an exogenous gene to transfer the exogenous gene (as a vehicle or shuttle) into a cell.

As used herein, an “expression vector” includes a vector capable of expressing a DNA, the DNA is operably linked to regulatory sequences, such as a promoter region, capable of affecting the expression of such a DNA fragment. Such additional fragments may include promoter and terminator sequences and optionally may include one or more origins of replication, one or more selectable markers, enhancers, polyadenylation signals, and the like. Expression vectors are typically derived from plasmid or viral DNA, or may contain elements of both. Thus, an expression vector refers to a recombinant DNA or RNA construct, such as a plasmid, phage, recombinant virus, or other vector. When introduced into an appropriate host cell, it results in the expression of a cloned DNA. Suitable expression vectors are well known to those skilled in the art and include expression vectors that are replicable in eukaryotic and/or prokaryotic cells and expression vectors that remain episomal or that are integrated into the host cell genome.

As used herein, the “treatment” of an individual with a disease or disease condition indicates that the symptoms of the individual are partially or completely relieved, or remain unchanged after treatments. Therefore, treatment includes prevention, treatment, and/or cure. Prevention refers to preventing the underlying disease and/or preventing the worsening of symptoms or the progression of the disease. Treatment also includes any pharmaceutical use of any antibody or antigen-binding fragment thereof provided and the compositions provided herein.

As used herein, “therapeutic effect” refers to an effect resulting from the treatment of a subject that alters, typically ameliorates, or ameliorates a symptom of a disease or condition, or cures a disease or condition.

As used herein, a “therapeutically effective amount” or “therapeutically effective dose” refers to an amount of a substance, compound, material, or composition comprising a compound that is at least sufficient to produce a therapeutic effect after administration to a subject. Thus, it is an amount necessary to prevent, cure, ameliorate, block, or partially block the symptoms of a disease or disorder.

As used herein, a “prophylactically effective amount” or “prophylactically effective dose” refers to an amount of a substance, compound, material, or composition comprising a compound that, when administered to a subject, has the desired prophylactic effect, e.g. prevents or delays the occurrence or recurrence of a disease or condition, reduces the likelihood of the occurrence or recurrence of a disease or condition. A fully prophylactically effective dose does not need to occur by administration of one dose, and may occur only after administration of a series of doses. Thus, a prophylactically effective amount can be administered in one or more administrations.

As used herein, the term “patient” refers to a mammal, such as a human.

### II. Detailed Description of Specific Embodiments

In one aspect, the present disclosure provides a bispecific antibody or antigen-binding portion thereof, comprising:
(a) a first antigen-binding portion or an antigen-binding fragment thereof bound to a BCMA antigen on the surface of a target cell, the first antigen-binding portion comprises a first heavy chain and a first light chain, the first antigen-binding portion comprises a first binding domain that binds to the first antigen, wherein the first binding domain comprises a heavy chain CDR selected from the amino acid sequences of SEQ ID NOs: 12, 13, 14, 26, 27, 28, 36, 37, 43, 44, 50, 55, 56, 57, 65, 66, 71, 72, 73 147, and 186, or any variant thereof, and/or a light chain CDR selected from the amino acid sequences of SEQ ID NOs: 17, 18, 19, 22, 23, 31, 32, 33, 40, 47, 60, 61, 62, 76, 77, 78, 83, 84, 87, 88, 89, 92 and 144 or any variant; and
(b) a second antigen-binding portion or antigen-binding fragment thereof bound to a CD3 antigen on the surface of a T cell, the second antigen-binding portion comprises a second heavy chain and a second light chain, the second antigen-binding portion comprises a second binding domain that binds to a second antigen.

The antibody or antigen-binding portion thereof according to the preceding aspect, the first binding domain comprises a heavy chain CDR1 selected from the amino acid sequences of SEQ ID NOs: 12, 26, 55, 65, 71 or any variant thereof, a heavy chain CDR2 selected from the amino acid sequences of SEQ ID NOs: 13, 27, 36, 43, 50, 56, 66, 72, 147, 186 or any variant thereof, a heavy chain CDR3 selected from the amino acid sequences of SEQ ID NOs: 14, 28, 37, 44, 57, 73 or any variant thereof; and/or the first binding domain comprises a light chain CDR1 selected from the amino acid sequences of SEQ ID NOs: 17, 22, 31, 47, 60, 76, 83, 87, 92, 144 or any variant thereof, a light chain CDR2 selected from the amino acid sequences of SEQ ID NOs: 18, 23, 32, 40, 61, 77, 84, 88 or any variant thereof, a light chain CDR3 selected from the amino acid sequences of SEQ ID NOs: 19, 33, 62, 78, 89 or any variant thereof.

The antibody or antigen-binding portion thereof according to any of the preceding aspects, the heavy chain CDR1, CDR2, and CDR3 sequences of the first binding domain are selected from: the first heavy chain CDR1, CDR2, and CDR3 sequences of the amino acid sequences of SEQ ID NOs: 12, 13 and 14, the first heavy chain CDR1, CDR2, and CDR3 sequences of the amino acid sequences of SEQ ID NOs: 12, 186 and 14, the first heavy chain CDR1, CDR2, and CDR3 sequences of the amino acid sequences of SEQ ID NOs: 26, 27 and 28, the first heavy chain CDR1, CDR2, and CDR3 sequences of the amino acid sequences of SEQ ID NOs: 12, 36 and 37, the first heavy chain CDR1, CDR2, and CDR3 sequences of the amino acid sequences of SEQ ID NOs: 12, 43 and 44, the first heavy chain CDR1, CDR2, and CDR3 sequences of the amino acid sequences of SEQ ID NOs: 12, 50 and 14, the first heavy chain CDR1, CDR2, and CDR3 sequences of the amino acid sequences of SEQ ID NOs: 55, 56 and 57, the first heavy chain CDR1, CDR2, and CDR3 sequences of the amino acid sequences of SEQ ID NOs: 65, 66 and 14, the first heavy chain CDR1, CDR2, and CDR3 sequences of the amino acid sequences of SEQ ID NOs: 71, 72 and 73; the first heavy chain CDR1, CDR2, and CDR3 sequences of the amino acid sequences of SEQ ID NOs: 71, 147 and 73; and the light chain CDR1, CDR2, and CDR3 sequences of the first binding domain are selected from: the sequences of the first light chain CDR1, CDR2 and CDR3 of the amino acid sequences of SEQ ID NOs: 17, 18 and 19, the sequences of the first light chain CDR1, CDR2 and CDR3 of the amino acid sequences of SEQ ID NOs: 22, 23 and 19, the sequences of the first light chain CDR1, CDR2 and CDR3 of the amino acid sequences of SEQ ID NOs: 31, 32 and 33, the sequences of the first light chain CDR1, CDR2 and CDR3 of the amino acid sequences of SEQ ID NOs: 17, 40 and 19, the sequences of the first light chain CDR1, CDR2 and CDR3 of the amino acid sequences of SEQ ID NOs: 47, 18 and 19, the sequences of the first light chain CDR1, CDR2 and CDR3 of the amino acid sequences of SEQ ID NOs: 60, 61 and 62, the sequences of the first light chain CDR1, CDR2 and CDR3 of the amino acid sequences of SEQ ID NOs: 76, 77 and 78, and the sequences of the first light chain CDR1, CDR2 and CDR3 of the amino acid sequences of SEQ ID NOs: 83, 84 and 19, the first light chain CDR1, CDR2, and CDR3 sequences of the amino acid sequences of SEQ ID NOs: 87, 88 and 89, the first light chain CDR1, CDR2, and CDR3 sequences of the amino acid sequences of SEQ ID NOs: 92, 23 and 19, the first light chain CDR1, CDR2, and CDR3 sequences of the amino acid sequences of SEQ ID NOs: 144, 77 and 78.

In some embodiments, the first binding domain comprises the first heavy chain CDR1, CDR2, and CDR3 sequences of the amino acid sequences of SEQ ID NOs: 12, 13 and 14 and the first light chain CDR1, CDR2, and CDR3 sequences of the amino acid sequences of SEQ ID NOs: 17, 18 and 19.

In some embodiments, the first binding domain comprises the first heavy chain CDR1, CDR2, and CDR3 sequences of the amino acid sequences of SEQ ID NOs: 12, 13 and 14 and the first light chain CDR1, CDR2, and CDR3 sequences of the amino acid sequences of SEQ ID NOs: 22, 23 and 19.

In some embodiments, the first binding domain comprises the first heavy chain CDR1, CDR2, and CDR3 sequences of the amino acid sequences of SEQ ID NOs: 26, 27 and 28 and the first light chain CDR1, CDR2, and CDR3 sequences of the amino acid sequences of SEQ ID NOs: 31, 32 and 33.

In some embodiments, the first binding domain comprises the first heavy chain CDR1, CDR2, and CDR3 sequences of the amino acid sequences of SEQ ID NOs: 12, 36 and 37 and the first light chain CDR1, CDR2, and CDR3 sequences of the amino acid sequences of SEQ ID NOs: 17, 40 and 19.

In some embodiments, the first binding domain comprises the first heavy chain CDR1, CDR2, and CDR3 sequences of the amino acid sequences of SEQ ID NOs: 12, 43 and 44 and the first light chain CDR1, CDR2, and CDR3 sequences of the amino acid sequences of SEQ ID NOs: 47, 18 and 19.

In some embodiments, the first binding domain comprises the first heavy chain CDR1, CDR2, and CDR3 sequences of the amino acid sequences of SEQ ID NOs: 12, 50 and 14 and the first light chain CDR1, CDR2, and CDR3 sequences of the amino acid sequences of SEQ ID NOs: 17, 40 and 19.

In some embodiments, the first binding domain comprises the first heavy chain CDR1, CDR2, and CDR3 sequences of the amino acid sequences of SEQ ID NOs: 55, 56 and 57 and the first light chain CDR1, CDR2, and CDR3 sequences of the amino acid sequences of SEQ ID NOs: 60, 61 and 62.

In some embodiments, the first binding domain comprises the first heavy chain CDR1, CDR2, and CDR3 sequences of the amino acid sequences of SEQ ID NOs: 12, 13 and 14 and the first light chain CDR1, CDR2, and CDR3 sequences of the amino acid sequences of SEQ ID NOs: 17, 40 and 19.

In some embodiments, the first binding domain comprises the first heavy chain CDR1, CDR2, and CDR3 sequences of the amino acid sequences of SEQ ID NOs: 65, 66 and 14 and the first light chain CDR1, CDR2, and CDR3 sequences of the amino acid sequences of SEQ ID NOs: 17, 40 and 19.

In some embodiments, the first binding domain comprises the first heavy chain CDR1, CDR2, and CDR3 sequences of the amino acid sequences of SEQ ID NOs: 71, 72 and 73 and the first light chain CDR1, CDR2, and CDR3 sequences of the amino acid sequences of SEQ ID NOs: 76, 77 and 78.

In some embodiments, the first binding domain comprises the first heavy chain CDR1, CDR2, and CDR3 sequences of the amino acid sequences of SEQ ID NOs: 12, 13 and 14 and the first light chain CDR1, CDR2, and CDR3 sequences of the amino acid sequences of SEQ ID NOs: 83, 84 and 19.

In some embodiments, the first binding domain comprises the first heavy chain CDR1, CDR2, and CDR3 sequences of the amino acid sequences of SEQ ID NOs: 12, 13, and 14 and the first light chain CDR1, CDR2, and CDR3 sequences of the amino acid sequences of SEQ ID NOs: 87, 88 and 89.

In some embodiments, the first binding domain comprises the first heavy chain CDR1, CDR2, and CDR3 sequences of the amino acid sequences of SEQ ID NOs: 12, 13 and 14 and the first light chain CDR1, CDR2, and CDR3 sequences of the amino acid sequences of SEQ ID NOs: 92, 23 and 19.

In some embodiments, the first binding domain comprises the first heavy chain CDR1, CDR2, and CDR3 sequences of the amino acid sequences of SEQ ID NOs: 71, 147 and 73 and the first light chain CDR1, CDR2, and CDR3 sequences of the amino acid sequences of SEQ ID NOs: 144, 77 and 78.

In some embodiments, the first binding domain comprises first heavy chain CDR1, CDR2, and CDR3 sequences of the amino acid sequences of SEQ ID NOs: 12, 186 and 14 and first light chain CDR1, CDR2, and CDR3 sequences of the amino acid sequences of SEQ ID NOs: 17, 40 and 19.

In some embodiments, the first binding domain comprises first heavy chain CDR1, CDR2, and CDR3 sequences of the amino acid sequences of SEQ ID NOs: 12, 186 and 14 and first light chain CDR1, CDR2, and CDR3 sequences of the amino acid sequences of SEQ ID NOs: 22, 23 and 19.

The antibody or antigen-binding portion thereof according to any of the preceding aspects, the first binding domain comprises a first heavy chain variable region selected from the amino acid sequences of SEQ ID NOs: 10, 24, 34, 41, 48, 53, 63, 69, 79, 145, 150, 154, 158, 162, 166, 170 and 187, or any variant thereof, and/or comprises a first light chain variable region selected from the amino acid sequences of SEQ ID NOs: 15, 20, 29, 38, 45, 51, 58, 67, 74, 81, 85, 90, 142, 148, 152, 156, 160, 164, 168, and 188, or any variant thereof.

In some embodiments, the first binding domain comprises a first heavy chain variable region of the amino acid sequence of SEQ ID NO: 10 or any variant thereof, and a first light chain variable region of the amino acid sequence of SEQ ID NO: 15 or any variant thereof.

In some embodiments, the first binding domain comprises a first heavy chain variable region of the amino acid sequence of SEQ ID NO: 10 or any variant thereof, and a first light chain variable region of the amino acid sequence of SEQ ID NO: 20 or any variant thereof.

In some embodiments, the first binding domain comprises a first heavy chain variable region of the amino acid sequence of SEQ ID NO: 24 or any variant thereof, and a first light chain variable region of the amino acid sequence of SEQ ID NO: 29 or any variant thereof.

In some embodiments, the first binding domain comprises a first heavy chain variable region of the amino acid sequence of SEQ ID NO: 34 or any variant thereof, and a first light chain variable region of the amino acid sequence of SEQ ID NO: 38 or any variant thereof.

In some embodiments, the first binding domain comprises a first heavy chain variable region of the amino acid sequence of SEQ ID NO: 41 or any variant thereof, and a first light chain variable region of the amino acid sequence of SEQ ID NO: 45 or any variant thereof.

In some embodiments, the first binding domain comprises a first heavy chain variable region of the amino acid sequence of SEQ ID NO: 48 or any variant thereof, and a first light chain variable region of the amino acid sequence of SEQ ID NO: 51 or any variant thereof.

In some embodiments, the first binding domain comprises a first heavy chain variable region of the amino acid sequence of SEQ ID NO: 53 or any variant thereof, and a first light chain variable region of the amino acid sequence of SEQ ID NO: 58 or any variant thereof.

In some embodiments, the first binding domain comprises a first heavy chain variable region of the amino acid sequence of SEQ ID NO: 10 or any variant thereof, and a first light chain variable region of the amino acid sequence of SEQ ID NO: 38 or any variant thereof.

In some embodiments, the first binding domain comprises a first heavy chain variable region of the amino acid sequence of SEQ ID NO: 63 or any variant thereof, and a first light chain variable region of the amino acid sequence of SEQ ID NO: 67 or any variant thereof.

In some embodiments, the first binding domain comprises a first heavy chain variable region of the amino acid sequence of SEQ ID NO: 69 or any variant thereof, and a first light chain variable region of the amino acid sequence of SEQ ID NO: 74 or any variant thereof.

In some embodiments, the first binding domain comprises a first heavy chain variable region of the amino acid sequence of SEQ ID NO: 79 or any variant thereof, and a first light chain variable region of the amino acid sequence of SEQ ID NO: 81 or any variant thereof.

In some embodiments, the first binding domain comprises a first heavy chain variable region of the amino acid sequence of SEQ ID NO: 10 or any variant thereof, and a first light chain variable region of the amino acid sequence of SEQ ID NO: 85 or any variant thereof.

In some embodiments, the first binding domain comprises a first heavy chain variable region of the amino acid sequence of SEQ ID NO: 10 or any variant thereof, and a first light chain variable region of the amino acid sequence of SEQ ID NO: 90 or any variant thereof.

In some embodiments, the first binding domain comprises a first heavy chain variable region of the amino acid sequence of SEQ ID NO: 145 or any variant thereof, and a first light chain variable region of the amino acid sequence of SEQ ID NO: 142 or any variant thereof.

In some embodiments, the first binding domain comprises a first heavy chain variable region of the amino acid sequence of SEQ ID NO: 150 or any variant thereof, and a first light chain variable region of the amino acid sequence of SEQ ID NO: 148 or any variant thereof.

In some embodiments, the first binding domain comprises a first heavy chain variable region of the amino acid sequence of SEQ ID NO: 154 or any variant thereof, and a first light chain variable region of the amino acid sequence of SEQ ID NO: 152 or any variant thereof.

In some embodiments, the first binding domain comprises a first heavy chain variable region of the amino acid sequence of SEQ ID NO: 158 or any variant thereof, and a first light chain variable region of the amino acid sequence of SEQ ID NO: 156 or any variant thereof.

In some embodiments, the first binding domain comprises a first heavy chain variable region of the amino acid sequence of SEQ ID NO: 162 or any variant thereof, and a first light chain variable region of the amino acid sequence of SEQ ID NO: 160 or any variant thereof.

In some embodiments, the first binding domain comprises a first heavy chain variable region of the amino acid sequence of SEQ ID NO: 166 or any variant thereof, and a first light chain variable region of the amino acid sequence of SEQ ID NO: 164 or any variant thereof.

In some embodiments, the first binding domain comprises a first heavy chain variable region of the amino acid sequence of SEQ ID NO: 170 or any variant thereof, and a first light chain variable region of the amino acid sequence of SEQ ID NO: 168 or any variant thereof.

In some embodiments, the first binding domain comprises a first heavy chain variable region of the amino acid sequence of SEQ ID NO: 187 or any variant thereof, and a first light chain variable region of the amino acid sequence of SEQ ID NO: 188 or any variant thereof.

The antibody or antigen-binding portion thereof according to any of the preceding aspects, the second binding domain comprises a heavy chain CDR selected from the amino acid sequences of SEQ ID NOs: 114, 115, 116, 119, 122, 125, 128, 131, 134, 135 or any variant thereof; and/or a light chain CDR selected from the amino acid sequences of SEQ ID NOs: 95, 96, 97, 102, 103, 108, 111 or any variant thereof.

In some embodiments, the second binding domain comprises a second heavy chain CDR1 selected from the amino acid sequences of SEQ ID NOs: 114, 119, 134 or any variant thereof, a second heavy chain CDR2 selected from the amino acid sequences of SEQ ID NOs: 115,135 or any variant thereof, a second heavy chain CDR3 selected from the amino acid sequences of SEQ ID NOs: 116, 122, 125, 128, 131 or any variant thereof; and/or a second light chain CDR1 selected from the amino acid sequences of SEQ ID NOs: 95, 102 or any variant thereof, a second light chain CDR2 selected from the amino acid sequences of SEQ ID NOs: 96, 103, 111 or any variant thereof, a second light chain CDR3 selected from the amino acid sequences of SEQ ID NOs: 91, 108 or any variant thereof.

In some embodiments, the heavy chain CDR1, CDR2, and CDR3 sequences of the second binding domain are selected from: second heavy chain CDR1, CDR2, and CDR3 sequences of the amino acid sequences of SEQ ID NOs: 114, 115 and 116, second heavy chain CDR1, CDR2, and CDR3 sequences of the amino acid sequences of SEQ ID NOs: 119, 115 and 116, second heavy chain CDR1, CDR2, and CDR3 sequences of the amino acid sequences of SEQ ID NOs: 119, 115 and 122, second heavy chain CDR1, CDR2, and CDR3 sequences of the amino acid sequences of SEQ ID NOs: 119, 115 and 125, second heavy chain CDR1, CDR2, and CDR3 sequences of the amino acid sequences of SEQ ID NOs: 119, 115 and 128, second heavy chain CDR1, CDR2, and CDR3 sequences of the amino acid sequences of SEQ ID NOs: 119, 115 and 131, second heavy chain CDR1, CDR2, and CDR3 sequences of the amino acid sequences of SEQ ID NOs: 134, 135 and 116; and the light chain CDR1, CDR2, and CDR3 sequences of the second binding domain are selected from: second light chain CDR1, CDR2, and CDR3 sequences of the amino acid sequences of SEQ ID NOs: 95, 96 and 97, second light chain CDR1, CDR2, and CDR3 sequences of the amino acid sequences of SEQ ID NOs: 102, 103 and 97, second light chain CDR1, CDR2, and CDR3 sequences of the amino acid sequences of SEQ ID NOs: 95, 96 and 108, second light chain CDR1, CDR2, and CDR3 sequences of the amino acid sequences of SEQ ID NOs: 95, 111 and 108.

In some embodiments, the second binding domain comprises the second heavy chain CDR1, CDR2, and CDR3 sequences of the amino acid sequences of SEQ ID NOs: 114, 115 and 116, and the second light chain CDR1, CDR2, and CDR3 sequences of the amino acid sequences of SEQ ID NOs: 95, 96 and 97.

In some embodiments, the second binding domain comprises the second heavy chain CDR1, CDR2, and CDR3 sequences of the amino acid sequences of SEQ ID NOs: 114, 115 and 116, and the second light chain CDR1, CDR2, and CDR3 sequences of the amino acid sequences of SEQ ID NOs: 102, 103 and 97.

In some embodiments, the second binding domain comprises the second heavy chain CDR1, CDR2, and CDR3 sequences of the amino acid sequences of SEQ ID NOs: 114, 115 and 116, and the second light chain CDR1, CDR2, and CDR3 sequences of the amino acid sequences of SEQ ID NOs: 95, 96 and 108.

In some embodiments, the second binding domain comprises the second heavy chain CDR1, CDR2, and CDR3 sequences of the amino acid sequences of SEQ ID NOs: 114, 115 and 116, and the second light chain CDR1, CDR2, and CDR3 sequences of the amino acid sequences of SEQ ID NOs: 95, 111 and 108.

In some embodiments, the second binding domain comprises a second heavy chain variable region selected from the amino acid sequences of SEQ ID NOs: 112, 117, 120, 123, 126, 129, 132, 136, 138, 140, or any variant thereof, and/or comprises a second light chain variable region selected from the amino acid sequences of SEQ ID NOs: 93, 98, 100, 104, 106, 109 or any variant thereof.

In some embodiments, the second binding domain comprises a second heavy chain variable region of the amino acid sequence of SEQ ID NO: 136 or any variant thereof, and a second light chain variable region of the amino acid sequence of SEQ ID NO: 93 or any variant thereof.

In some embodiments, the second binding domain comprises a second heavy chain variable region of the amino acid sequence of SEQ ID NO: 138 or any variant thereof, and a second light chain variable region of the amino acid sequence of SEQ ID NO: 93 or any variant thereof.

In some embodiments, the second binding domain comprises a second heavy chain variable region of the amino acid sequence of SEQ ID NO: 138 or any variant thereof, and a second light chain variable region of the amino acid sequence of SEQ ID NO: 98 or any variant thereof.

In some embodiments, the second binding domain comprises a second heavy chain variable region of the amino acid sequence of SEQ ID NO: 138 or any variant thereof, and a second light chain variable region of the amino acid sequence of SEQ ID NO: 100 or any variant thereof.

In some embodiments, the second binding domain comprises a second heavy chain variable region of the amino acid sequence of SEQ ID NO: 112 or any variant thereof, and a second light chain variable region of the amino acid sequence of SEQ ID NO: 106 or any variant thereof.

In some embodiments, the second binding domain comprises a second heavy chain variable region of the amino acid sequence of SEQ ID NO: 136 or any variant thereof, and a second light chain variable region of the amino acid sequence of SEQ ID NO: 106 or any variant thereof.

In some embodiments, the second binding domain comprises a second heavy chain variable region of the amino acid sequence of SEQ ID NO: 138 or any variant thereof, and a second light chain variable region of the amino acid sequence of SEQ ID NO: 106 or any variant thereof.

The antibody or antigen-binding portion thereof according to any of the preceding aspects, the first light chain of the first antigen-binding portion is a κ light chain and the second light chain of the second antigen-binding portion is a λ light chain.

In some embodiments, the second light chain variable region of the second antigen-binding portion has a Gln₄₀Glu mutation (Vλ_{CD3}:Gln₄₀Glu); the second heavy chain variable region of the second antigen-binding portion has a Gln₉Lys mutation (VH_{CD3}:Gln₃₉Lys).

In some embodiments, the first light chain variable region of the first antigen-binding portion has a Gln₄₂Lys mutation (Vκ_{BCMA}:Gln₄₂Lys) In some embodiments, the first heavy chain variable region of the first antigen-binding portion has a Gln₃₉Glu mutation (VH_{BCMA}:Gln₃₉Glu).

In some embodiments, the Fc portion of the first antigen-binding portion and the second antigen-binding portion of the bispecific antibody adopts a knob-into-hole structure. In some preferred embodiments, the Fc portion adopts a knob-into-hole structure of human IgG4.

In some embodiments, the first heavy chain comprises a heavy chain selected from SEQ ID NOs: 174, 178, or any variant thereof, the first light chain comprises a light chain selected from SEQ ID Nos: 172, 176, or any variant thereof.

In some preferred embodiments, the first heavy chain comprises a heavy chain selected from SEQ ID NO: 174 or any variant thereof, the first light chain comprises a light chain selected from SEQ ID NO: 172 or any variant thereof.

In some preferred embodiments, the first heavy chain comprises a heavy chain selected from SEQ ID NO: 174 or any variant thereof, the first light chain comprises a light chain selected from SEQ ID NO: 176 or any variant thereof.

In some preferred embodiments, the first heavy chain comprises a heavy chain selected from SEQ ID NO: 178 or any variant thereof, the first light chain comprises a light chain selected from SEQ ID NO: 172 or any variant thereof.

In some preferred embodiments, the first heavy chain comprises a heavy chain selected from SEQ ID NO: 178 or any variant thereof, the first light chain comprises a light chain selected from SEQ ID NO: 176 or any variant thereof.

In some embodiments, the second heavy chain comprises a heavy chain of SEQ ID NO: 182 or any variant thereof, the second light chain comprises a light chain of SEQ ID NO: 180 or any variant thereof.

In some preferred embodiments, the first binding domain of the bispecific antibody comprises a first heavy chain variable region of the amino acid sequence of SEQ ID NO: 145 or any variant thereof, and a first light chain variable region of the amino acid sequence of SEQ ID NO: 142 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof.

In some preferred embodiments, the first binding domain of the bispecific antibody comprises a first heavy chain variable region of the amino acid sequence of SEQ ID NO: 150 or any variant thereof, and a first light chain variable region of the amino acid sequence of SEQ ID NO: 148 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof.

In some preferred embodiments, the first binding domain of the bispecific antibody comprises a first heavy chain variable region of the amino acid sequence of SEQ ID NO: 154 or any variant thereof, and a first light chain variable region of the amino acid sequence of SEQ ID NO: 152 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof.

In some preferred embodiments, the first binding domain of the bispecific antibody comprises a first heavy chain variable region of the amino acid sequence of SEQ ID NO: 158 or any variant thereof, and a first light chain variable region of the amino acid sequence of SEQ ID NO: 156 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof.

In some preferred embodiments, the first binding domain of the bispecific antibody comprises a first heavy chain variable region of the amino acid sequence of SEQ ID NO: 162 or any variant thereof, and a first light chain variable region of the amino acid sequence of SEQ ID NO: 160 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof.

In some preferred embodiments, the first binding domain of the bispecific antibody comprises a first heavy chain variable region of the amino acid sequence of SEQ ID NO: 166 or any variant thereof, and a first light chain variable region of the amino acid sequence of SEQ ID NO: 164 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof.

In some preferred embodiments, the first binding domain of the bispecific antibody comprises a first heavy chain variable region of the amino acid sequence of SEQ ID NO: 170 or any variant thereof, and a first light chain variable region of the amino acid sequence of SEQ ID NO: 168 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof; In a specific embodiment, the first heavy chain comprises SEQ ID NO: 174, the first light chain of the bispecific antibody comprises SEQ ID NO: 172; the second heavy chain comprises SEQ ID NO: 182, the second light chain comprises SEQ ID NO: 180.

In a specific embodiment, the first heavy chain comprises SEQ ID NO: 174, the first light chain of the bispecific antibody comprises SEQ ID NO: 176; the second heavy chain comprises SEQ ID NO: 182, the second light chain comprises SEQ ID NO: 180.

In a specific embodiment, the first heavy chain comprises SEQ ID NO: 178, the first light chain of the bispecific antibody comprises SEQ ID NO: 172; the second heavy chain comprises SEQ ID NO: 182, the second light chain comprises SEQ ID NO: 180.

In a specific embodiment, the first heavy chain comprises SEQ ID NO: 178, the first light chain of the bispecific antibody comprises SEQ ID NO: 176; the second heavy chain comprises SEQ ID NO: 182, the second light chain comprises SEQ ID NO: 180.

In one aspect, the present disclosure provides a BCMA-binding antibody or antigen-binding portion thereof, the antibody comprises a first antigen-binding portion as previously described.

In some embodiments, the BCMA-binding antibody or antigen-binding portion thereof has at least greater than 60%, 65%, 70%, 75%, 80%, 85%, 90%, 95%, 96%, 97%, 98%, 99% or more sequence identity to the antibody or antigen-binding portion thereof of any of the preceding aspects.

In one aspect, the present disclosure provides a nucleic acid encoding the antibody or antigen-binding portion thereof according to any of the preceding aspects, or a nucleic acid molecule having at least greater than 60%, 65%, 70%, 75%, 80%, 85%, 90%, 95%, 96%, 97%, 98%, 99% or more sequence identity thereto.

In some embodiments, the nucleic acid encoding the first heavy chain variable region of the first antigen-binding portion is selected from the nucleotide sequences of SEQ ID NOs: 11, 25, 35, 42, 49, 54, 64, 70, 80, 146, 151, 155, 159, 163, 167, 171, and 189 or any variant thereof; and/or the nucleic acid encoding the first light chain variable region of the first antigen-binding portion is selected from the nucleotide sequences of SEQ ID NOs: 16, 21, 30, 39, 46, 52, 59, 68, 75, 82, 86, 91, 143, 149, 153, 157161, 165, 169, and 190 or any variant thereof.

In some preferred embodiments, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 16.

In some preferred embodiments, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 21.

In some preferred embodiments, the nucleic acid encoding the first heavy chain variable region of the first antigen-binding portion is the nucleotide sequence of SEQ ID NO: 25; and the nucleic acid encoding the first light chain variable region is the nucleotide sequence of SEQ ID NO: 30.

In some preferred embodiments, the nucleic acid encoding the first heavy chain variable region of the first antigen-binding portion is the nucleotide sequence of SEQ ID NO: 35; and the nucleic acid encoding the first light chain variable region is the nucleotide sequence of SEQ ID NO: 39.

In some preferred embodiments, the nucleic acid encoding the first heavy chain variable region of the first antigen-binding portion is the nucleotide sequence of SEQ ID NO: 42; and the nucleic acid encoding the first light chain variable region is the nucleotide sequence of SEQ ID NO: 46.

In some preferred embodiments, the nucleic acid encoding the first heavy chain variable region of the first antigen-binding portion is the nucleotide sequence of SEQ ID NO: 49; and the nucleic acid encoding the first light chain variable region is the nucleotide sequence of SEQ ID NO: 52.

In some preferred embodiments, the nucleic acid encoding the first heavy chain variable region of the first antigen-binding portion is the nucleotide sequence of SEQ ID NO: 54; and the nucleic acid encoding the first light chain variable region is the nucleotide sequence of SEQ ID NO: 59.

In some preferred embodiments, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 39.

In some preferred embodiments, the nucleic acid encoding the first heavy chain variable region of the first antigen-binding portion is the nucleotide sequence of SEQ ID NO: 64; and the nucleic acid encoding the first light chain variable region is the nucleotide sequence of SEQ ID NO: 68.

In some preferred embodiments, the nucleic acid encoding the first heavy chain variable region of the first antigen-binding portion is the nucleotide sequence of SEQ ID NO: 70; and the nucleic acid encoding the first light chain variable region is the nucleotide sequence of SEQ ID NO: 75.

In some preferred embodiments, the nucleic acid encoding the first heavy chain variable region of the first antigen-binding portion is the nucleotide sequence of SEQ ID NO: 80; and the nucleic acid encoding the first light chain variable region is the nucleotide sequence of SEQ ID NO: 82.

In some preferred embodiments, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 86.

In some preferred embodiments, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 91.

In some preferred embodiments, the nucleic acid encoding the first heavy chain variable region of the first antigen-binding portion is the nucleotide sequence of SEQ ID NO: 146; and the nucleic acid encoding the first light chain variable region is the nucleotide sequence of SEQ ID NO: 143.

In some preferred embodiments, the nucleic acid encoding the first heavy chain variable region of the first antigen-binding portion is the nucleotide sequence of SEQ ID NO: 151; and the nucleic acid encoding the first light chain variable region is the nucleotide sequence of SEQ ID NO: 149.

In some preferred embodiments, the nucleic acid encoding the first heavy chain variable region of the first antigen-binding portion is the nucleotide sequence of SEQ ID NO: 155; and the nucleic acid encoding the first light chain variable region is the nucleotide sequence of SEQ ID NO: 153.

In some preferred embodiments, the nucleic acid encoding the first heavy chain variable region of the first antigen-binding portion is the nucleotide sequence of SEQ ID NO: 159; and the nucleic acid encoding the first light chain variable region is the nucleotide sequence of SEQ ID NO: 157.

In some preferred embodiments, the nucleic acid encoding the first heavy chain variable region of the first antigen-binding portion is the nucleotide sequence of SEQ ID NO: 163; and the nucleic acid encoding the first light chain variable region is the nucleotide sequence of SEQ ID NO: 161.

In some preferred embodiments, the nucleic acid encoding the first heavy chain variable region of the first antigen-binding portion is the nucleotide sequence of SEQ ID NO: 167; and the nucleic acid encoding the first light chain variable region is the nucleotide sequence of SEQ ID NO: 165.

In some preferred embodiments, the nucleic acid encoding the first heavy chain variable region of the first antigen-binding portion is the nucleotide sequence of SEQ ID NO: 171; and the nucleic acid encoding the first light chain variable region is the nucleotide sequence of SEQ ID NO: 169.

In some preferred embodiments, the nucleic acid encoding the first heavy chain variable region of the first antigen-binding portion is the nucleotide sequence of SEQ ID NO: 189; and the nucleic acid encoding the first light chain variable region is the nucleotide sequence of SEQ ID NO: 190.

In some embodiments, the nucleic acid encoding the first heavy chain of the first antigen-binding portion is selected from the nucleotide sequences of SEQ ID NOs: 175, 179 or any variant thereof; and/or the nucleic acid encoding the first light chain of the first antigen-binding portion is selected from the nucleotide sequences of SEQ ID NOs: 173, 177 or any variant thereof.

In some preferred embodiments, the nucleic acid encoding the first heavy chain of the first antigen-binding portion is the nucleotide sequence of SEQ ID NO: 175, and the nucleic acid encoding the first light chain of the first antigen-binding portion is the nucleotide sequence of SEQ ID NO: 173.

In some preferred embodiments, the nucleic acid encoding the first heavy chain of the first antigen-binding portion is the nucleotide sequence of SEQ ID NO: 175, and the nucleic acid encoding the first light chain of the first antigen-binding portion is the nucleotide sequence of SEQ ID NO: 177.

In some preferred embodiments, the nucleic acid encoding the first heavy chain of the first antigen-binding portion is the nucleotide sequence of SEQ ID NO: 179 and the nucleic acid encoding the first light chain of the first antigen-binding portion is the nucleotide sequence of SEQ ID NO: 173.

In some preferred embodiments, the nucleic acid encoding the first heavy chain of the first antigen-binding portion is the nucleotide sequence of SEQ ID NO: 179 and the nucleic acid encoding the first light chain of the first antigen-binding portion is the nucleotide sequence of SEQ ID NO: 177.

In some embodiments, the nucleic acid encoding the second heavy chain variable region of the second antigen-binding portion is selected from the nucleotide sequences of SEQ ID NOs: 113, 118, 121, 124, 127, 130, 133, 137, 139, 141 or any variant thereof; and/or the nucleic acid encoding the second light chain variable region of the second antigen-binding portion is selected from the nucleotide sequences of SEQ ID NOs: 94, 99, 101, 105, 107, 110 or any variant thereof.

In some preferred embodiments, the nucleic acid encoding the second heavy chain variable region of the second antigen-binding portion is the nucleotide sequence of SEQ ID NO: 137; and the nucleic acid encoding the second light chain variable region is the nucleotide sequence of SEQ ID NO: 94.

In some preferred embodiments, the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 94.

In some preferred embodiments, the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 99.

In some preferred embodiments, the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 101.

In some preferred embodiments, the nucleic acid encoding the second heavy chain variable region of the second antigen-binding portion is the nucleotide sequence of SEQ ID NO: 113; and the nucleic acid encoding the second light chain variable region is the nucleotide sequence of SEQ ID NO: 107.

In some preferred embodiments, the nucleic acid encoding the second heavy chain variable region of the second antigen-binding portion is the nucleotide sequence of SEQ ID NO: 137; and the nucleic acid encoding the second light chain variable region is the nucleotide sequence of SEQ ID NO: 107.

In some preferred embodiments, the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107.

In some embodiments, the nucleic acid encoding the second heavy chain of the second antigen-binding portion is the nucleotide sequence of SEQ ID NO: 183 or any variant thereof; and/or the nucleic acid encoding the second light chain of the second antigen-binding portion is the nucleotide sequence of SEQ ID NO: 181 or any variant thereof.

In some preferred embodiments, the nucleic acid encoding the first heavy chain variable region of the first antigen-binding portion of the bispecific antibody is the nucleotide sequence of SEQ ID NO: 146; and the nucleic acid encoding the first light chain variable region is the nucleotide sequence of SEQ ID NO: 143; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107.

In some preferred embodiments, the nucleic acid encoding the first heavy chain variable region of the first antigen-binding portion of the bispecific antibody is the nucleotide sequence of SEQ ID NO: 151; and the nucleic acid encoding the first light chain variable region is the nucleotide sequence of SEQ ID NO: 149; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107.

In some preferred embodiments, the nucleic acid encoding the first heavy chain variable region of the first antigen-binding portion of the bispecific antibody is the nucleotide sequence of SEQ ID NO: 155; and the nucleic acid encoding the first light chain variable region is the nucleotide sequence of SEQ ID NO: 153; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107.

In some preferred embodiments, the nucleic acid encoding the first heavy chain variable region of the first antigen-binding portion of the bispecific antibody is the nucleotide sequence of SEQ ID NO: 159; and the nucleic acid encoding the first light chain variable region is the nucleotide sequence of SEQ ID NO: 157; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107.

In some preferred embodiments, the nucleic acid encoding the first heavy chain variable region of the first antigen-binding portion of the bispecific antibody is the nucleotide sequence of SEQ ID NO: 163; and the nucleic acid encoding the first light chain variable region is the nucleotide sequence of SEQ ID NO: 161; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107.

In some preferred embodiments, the nucleic acid encoding the first heavy chain variable region of the first antigen-binding portion of the bispecific antibody is the nucleotide sequence of SEQ ID NO: 167; and the nucleic acid encoding the first light chain variable region is the nucleotide sequence of SEQ ID NO: 165; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107.

In some preferred embodiments, the nucleic acid encoding the first heavy chain variable region of the first antigen-binding portion of the bispecific antibody is the nucleotide sequence of SEQ ID NO: 171; and the nucleic acid encoding the first light chain variable region is the nucleotide sequence of SEQ ID NO: 169; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107.

In some preferred embodiments, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181.

In some preferred embodiments, the nucleic acid encoding the first heavy chain of the first antigen-binding portion of the bispecific antibody is the nucleotide sequence of SEQ ID NO: 175, and the nucleic acid encoding the first light chain of the first antigen-binding portion is the nucleotide sequence of SEQ ID NO: 173, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181.

In some preferred embodiments, the nucleic acid encoding the first heavy chain of the first antigen-binding portion of the bispecific antibody is the nucleotide sequence of SEQ ID NO: 175, and the nucleic acid encoding the first light chain of the first antigen-binding portion is the nucleotide sequence of SEQ ID NO: 177, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181.

In some preferred embodiments, the nucleic acid encoding the first heavy chain of the first antigen-binding portion of the bispecific antibody is the nucleotide sequence of SEQ ID NO: 179, and the nucleic acid encoding the first light chain of the first antigen-binding portion is the nucleotide sequence of SEQ ID NO: 173, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181.

In some preferred embodiments, the nucleic acid encoding the first heavy chain of the first antigen-binding portion of the bispecific antibody is the nucleotide sequence of SEQ ID NO: 179, and the nucleic acid encoding the first light chain of the first antigen-binding portion is the nucleotide sequence of SEQ ID NO: 177, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181.

In one aspect, the present disclosure provides a BCMA-binding antibody or antigen-binding portion thereof, the antibody comprises a first antigen-binding portion as previously described.

In one aspect, the present disclosure provides a vector comprising the foregoing nucleic acids.

In one aspect, the present disclosure provides a cell comprising the foregoing nucleic acids or vector.

In one aspect, the present disclosure provides compositions comprising the foregoing bispecific antibodies or antigen-binding portions thereof, nucleic acids, vectors, and/or cells.

In one aspect, the present disclosure provides an antibody-drug conjugate comprising the foregoing bispecific antibody or antigen-binding portion thereof covalently attached to a therapeutic moiety.

Preferably, the therapeutic moiety is selected from a cytotoxic moiety, a chemotherapeutic agent, a cytokine, an immunosuppressant, an immunostimulant, a lytic peptide, or a radioisotope.

The antibodies of the present disclosure are useful as therapeutic or diagnostic tools in a variety of diseases in which BCMA is adversely expressed or found.

In one embodiment of a disease associated with BCMA, the expression of BCMA in cells of the diseased tissue or organ is increased, as compared to the state in a healthy tissue or organ. An increase means an increase of at least 10%, in particular at least 20%, at least 50%, at least 100%, at least 200%, at least 500%, at least 1000%, at least 10000%, or even more. In one embodiment, expression is found only in diseased tissues, whereas expression in corresponding healthy tissues is suppressed. According to the present disclosure, diseases associated with BCMA include B-cell diseases, e.g. B-cell disorders such as plasma cell disorders and/or autoimmune diseases.

In some embodiments, the disease condition is cancer. In some embodiments, the cancer is a B-cell related cancer selected from the group consisting of multiple myeloma, malignant plasmacytoma, Hodgkin lymphoma, nodular lymphocyte predominant Hodgkin lymphoma, kahler's disease and myeloid leukemia, plasma cell leukemia, plasmacytoma, B-cell prolymphocytic leukemia, hairy cell leukemia, B-cell non-Hodgkin lymphoma (NHL), acute myelogenous leukemia (AML), chronic lymphocytic leukemia (CLL), acute lymphocytic leukemia (ALL), chronic myelogenous leukemia (CML), follicular lymphoma, burkitt lymphoma, marginal zone lymphoma, mantle cell lymphoma, large cell lymphoma, precursor B-lymphocyte lymphoma, myeloid leukemia, waldenstrom's macroglobulinaemia, diffuse large B cell lymphoma, follicular lymphoma, marginal zone lymphoma, mucosa-associated lymphoid tissue lymphoma, small cell lymphocytic lymphoma, mantle cell lymphoma, burkitt lymphoma, primary mediastinal (thymic) large B cell lymphoma, lymphoplasmacytic lymphoma, waldenstrom's macroglobulinaemia, nodal marginal zone B cell lymphoma, splenic marginal zone lymphoma, intravascular large B-cell lymphoma, primary effusion lymphoma, lymphomatoid granulomatosis, large B-cell lymphoma rich in T cells/histiocytes, primary central nervous system lymphoma, primary cutaneous diffuse large B-cell lymphoma (leg type), EBV-positive diffuse large B-cell lymphoma of the elderly, diffuse large B-cell lymphoma associated with inflammation, intravascular large B-cell lymphoma, ALK-positive large B-cell lymphoma, plasmablastic lymphoma, large B-cell lymphoma arising in HHV8-associated multicenter Castleman's disease, unclassified B-cell lymphoma with intermediate features between diffuse large B-cell lymphoma and Burkitt's lymphoma, unclassified B-cell lymphoma with intermediate features between diffuse large B-cell lymphoma and classic Hodgkin lymphoma, and other B-cell-related lymphomas.

In plasma cell disorders, one plasma cell clone reproduces uncontrollably. As a result, this clone produces a large number of single (monoclonal) antibodies called M-proteins. In some cases, for example, a subject with monoclonal gammopathy, the antibody produced is incomplete, consisting only of light chains or heavy chains. These abnormal plasma cells and the produced antibodies thereof are generally limited to one type. Preferably, the plasma cell disorder is selected from multiple myeloma, plasmacytoma, plasma cell leukemia, macroglobulinaemia, amyloidosis, waldenstrom's macroglobulinaemia, solitary plasmacytoma of the bone, extramedullary plasmacytoma, osteosclerotic myeloma, heavy chain disease, monoclonal gammopathy of indetermined significance, and multiple myeloma of stasis type.

In some embodiments, the disease condition is an autoimmune disorder, such as systemic lupus erythematosus or rheumatoid arthritis. In some embodiments, the therapeutic agent comprises an antibody that specifically binds to an activated T-cell antigen.

In one embodiment, the therapeutic agent comprises an antibody that specifically binds to CD3, particularly CD3ε.

Methods of treating diseases and conditions using bispecific antibodies of the present disclosure include the steps of administering to a mammal a therapeutically effective amount of an antibody or antigen-binding fragment thereof or a nucleic acid molecule or vector or cell or pharmaceutical composition of any of the preceding aspects.

In some embodiments, the present disclosure provides a method of treating or preventing a cancer disease comprising administering to a patient an antibody capable of binding to BCMA, wherein the antibody is administered to provide a serum level of at least 40 μg/ml. In various embodiments, the antibody is administered to provide a serum level of at least 50 μg/ml, at least 150 μg/ml, at least 300 μg/ml, at least 400 μg/ml, or at least 500 μg/ml. In various embodiments, the antibody is administered to provide a serum level of no more than 800 μg/ml, 700 μg/ml, 600 μg/ml, 550 μg/ml, or 500 μg/ml. In one embodiment, the serum level provided is from 40 μg/ml to 700 μg/ml, preferably from 40 μg/ml to 600 μg/ml, preferably from 50 μg/ml to 500 μg/ml, such as from 150 μg/ml to 500 μg/ml or from 300 μg/ml to 500 μg/ml. The term “serum level” as used in the present description means the concentration of the discussed substance in serum. In one embodiment, serum levels are provided for at least 7 days or at least 14 days. In one embodiment, the method comprises administering a dose of the antibody of at least 300 mg/m², such as at least 600 mg/m², and preferably at most 1500 mg/m², at most 1200 mg/m², or at most 1000 mg/m².

In some embodiments, the disclosure provides a method of treating or preventing a cancer disease, comprising administering to a patient an antibody capable of binding to BCMA, wherein the antibody is administered at a dose of at least 300 mg/m², such as at least 600 mg/m², and preferably at most 1500 mg/m², at most 1200 mg/m², or at most 1000 mg/m².

In some embodiments, the disclosure provides a method of treating or preventing a cancer disease comprising administering to a patient an antibody capable of binding to BCMA, wherein at least 50%, preferably 60%, 70%, 80%, or 90% of the cancer cells of the patient are positive for BCMA and/or at least 40%, preferably 50% or 60% of the cancer cells of the patient are positive for surface expression of BCMA. In this aspect, the present disclosure also provides a method of treating or preventing a cancer disease, the method comprises: a. identifying patients showing at least 50%, preferably 60%, 70%, 80%, or 90% of BCMA-positive cancer cells and/or at least 40%, preferably 50% or 60% of cancer cells that are positive for surface expression of BCMA; and b. administering to the patient an antibody capable of binding to BCMA. In one embodiment, at least 95% or at least 98% of the cancer cells of the patient are BCMA-positive. In one embodiment, at least 70%, at least 80%, or at least 90% of the cancer cells of the patient are positive for surface expression of BCMA.

In one embodiment of the methods of any of the aspects described herein, the therapeutic outcome of the cancer disease is the achievement of stable disease conditions. In one embodiment, stable disease conditions are achieved for at least 2 months, at least 3 months, or at least 6 months.

In some embodiments, the present disclosure provides methods of achieving stable disease in a cancer patient comprising administering to a patient an antibody capable of binding to BCMA. In one embodiment, stable disease is achieved for at least 2 months, at least 3 months, or at least 6 months.

In one embodiment of the method of any of the aspects described herein, the antibody is administered in a single dose or multiple doses.

In some embodiments, the present disclosure provides a method of treating or preventing a cancer disease comprising administering to a patient an antibody capable of binding to BCMA, wherein the antibody is administered in multiple doses.

If the antibody is administered in multiple doses according to the present disclosure, the antibody is preferably administered in at least 3 doses, at least 4 doses, at least 5 doses, at least 6 doses, at least 7 doses, at least 8 doses, at least 9 doses, or at least 10 doses, and preferably at most 30 doses, 25 doses, 20 doses, 15 doses, or 10 doses. Preferably, the dose of antibody is administered at intervals of at least 7 days, at least 10 days, at least 14 days, or at least 20 days. The dose of antibody is preferably administered at intervals of from 7 to 30 days, from 10 to 20 days, and preferably about 14 days.

In one embodiment, the antibody is administered so as to provide a serum level of at least 40 μg/ml.

In various embodiments, the antibody is administered so as to provide a serum level of at least 50 μg/ml, at least 150 μg/ml, at least 300 μg/ml, at least 400 μg/ml, or at least 500 μg/ml. In various embodiments, the antibody is administered so as to provide a serum level of no more than 800 μg/ml, 700 μg/ml, 600 μg/ml, 550 μg/ml, or 500 μg/ml. In one embodiment, the serum level provided is from 40 μg/ml to 700 μg/ml, preferably from 40 μg/ml to 600 μg/ml, preferably from 50 μg/ml to 500 μg/ml, such as from 150 μg/ml to 500 μg/ml or from 300 μg/ml to 500 μg/ml. In one embodiment, serum levels are provided for at least 7 days or at least 14 days. In one embodiment, the method comprises administering a dose of at least 300 mg/m², such as at least 600 mg/m² and preferably at most 1500 mg/m², at most 1200 mg/m², or at most 1000 mg/m² of the antibody.

Use of an antibody or antigen-binding fragment thereof or a nucleic acid molecule or a vector or a cell or a pharmaceutical composition of any of the preceding aspects in the manufacture of a drug for treating a BCMA-related disorder in a mammal.

According to any of the preceding aspects, optionally, the antibody is conjugated to other drugs, such as a labeled or cytotoxic conjugate.

In one aspect, the disclosure also includes kits, e.g. kits comprising an antibody, fragment thereof, homologue, derivative, etc. of the disclosure, e.g. a labeled or cytotoxic conjugate, as well as instructions for use of the antibody, a conjugate that kills a particular type of cell, etc. The instructions can include directions for using the antibody, conjugate, etc. in vitro, in vivo, or ex vivo. The antibodies may be in liquid form or solid form, usually lyophilized. The kit may contain other suitable reagents, such as buffers, reconstitution solutions, and other necessary ingredients for the intended use. Combinations of reagents packaged in predetermined amounts with instructions for their use, e.g. for therapeutic use or for conducting diagnostic assays, are contemplated. When the antibody is labeled, e.g. with an enzyme, then the kit can include a substrate and cofactors required for the enzyme (e.g. a substrate precursor that provides a detectable chromophore or fluorophore).

In addition, other additives such as stabilizers, buffers (e.g. blocking buffers or lysis buffers), and the like may also be included. The relative amounts of the various reagents can be varied to provide a concentrate of reagent solution, thereby providing user flexibility, space savings, reagent savings, etc. These reagents may also be provided in dry powder form, usually in lyophilized form, including excipients which provide a reagent solution having the appropriate concentration when dissolved.

Use of an antibody or functional fragment thereof or a nucleic acid molecule or a vector or a cell or a pharmaceutical composition or kit according to any of the preceding aspects for the preparation of a reagent for inhibiting BCMA binding.

In addition, the antibodies of the present disclosure can be used in immunoassays, purification methods, and other methods using immunoglobulins or fragments thereof. Such uses are well-known in the art.

Accordingly, the present disclosure also provides compositions comprising the anti-BCMA antibodies of the present disclosure or fragments thereof, the antibody is conveniently combined with a pharmaceutically acceptable carrier, diluent, or excipient, which belongs to conventional practices in the art.

As used in the present disclosure, the term “pharmaceutical composition” refers to formulations of various prepared materials. Formulations containing a therapeutically effective amount of multivalent antibodies are in a sterile liquid solution, liquid suspension, or lyophilized form, optionally containing stabilizers or excipients.

The antibodies of the present disclosure can be used as a composition administered alone or can be used in combination with other active agents.

In some embodiments, the humanized antibodies of the disclosure are conjugated to a therapeutic moiety (i.e. a drug). The therapeutic moiety can be, for example, a cytotoxin, a chemotherapeutic agent, a cytokine, an immunosuppressant, an immunostimulant, a lytic peptide, or a radioisotope. Such conjugates are referred to herein as “antibody-drug conjugates” or “ADC”.

In some embodiments, the antibody is conjugated to a cytotoxic moiety. The cytotoxic moiety may for example be selected from the following: paclitaxel; cytochalasin B; gramicidin D; ethidium bromide; emetine; mitomycin; etoposide; teniposide; vincristine; vinblastine; colchicine; doxorubicin; daunorubicin; dihydroxy anthracenedione; tubulin inhibitors such as maytansine or analogs or derivatives thereof; antimitotic agents such as monomethyl auristatin E or F or analogues or derivatives thereof; dolastatin 10 or 15 or an analogue thereof; irinotecan or an analog thereof; mitoxantrone; mithramycin; actinomycin D; 1-dehydrotestosterone; glucocorticoids; procaine; tetracaine; lidocaine; propranolol; puromycin; calicheamicin or an analogue or derivative thereof; antimetabolites such as methotrexate, 6 mercaptopurine, 6 thioguanine, cytarabine, fludarabine, 5 fluorouracil, decadiazine, hydroxyurea, asparaginase, gemcitabine or cladribine; alkylating agents such as mechlorethamine, thiopurine, chlorambucil, melphalan, carmustine (BSNU), lomustine (CCNU), cyclophosphamide, busulfan, dibromomannitol, streptozotocin, dacarbazine (DTIC), procarbazine, mitomycin C; platinum derivatives such as cisplatin or carboplatin; duocarmycin A, duocarmycin SA, rachelmycin (CC-1065) or analogs or derivatives thereof; antibiotics such as actinomycin, bleomycin, daunorubicin, doxorubicin, idarubicin, mithramycin, mitomycin, mitoxantrone, plicomycin, anthramycin (AMC); pyrrolo [2,1-c][1,4]-benzodiazepine (PDB); diphtheria toxin and related molecules such as diphtheria A chain and active fragments thereof and hybrid molecules, ricin toxin such as ricin A or deglycosylated ricin A chain toxin, cholera toxin, Shiga-like toxin such as SLT I, SLT II, SLT IIV, LT toxin, C3 toxin, Shiga toxin, pertussis toxin, tetanus toxin, soybean Bowman-Birk protease inhibitor, pseudomonas exotoxin, azlocillin, saporin, modeccin, gelsolin, abrin A chain, modeccin A chain, α-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitomycin, restrictocin, phenomycin, and enomycin toxins; ribonuclease (RNase); DNase I, staphylococcal endotoxin A; pokeweed antiviral protein; diphtheria toxin and pseudomonas endotoxin.

In some embodiments, the antibody is conjugated to auristatin or a peptide analog, derivative, or prodrug thereof. It has been shown that auristatin interferes with microtubule dynamics, GTP hydrolysis, and nuclear and cell division and has anti-cancer and anti-fungal activity. For example, auristatin E can be reacted with p-acetylbenzoic acid or benzoylpentanoic acid to produce AEB and AEVB, respectively. Other typical auristatin derivatives include AFP, MMAF (monomethyl auristatin F), and MMAE (monomethyl auristatin E). Suitable auristatins and analogs, derivatives, and prodrugs of auristatins, as well as suitable linkers for conjugating auristatins to Ab, are described, for example, in U.S. Pat. Nos. 5,635,483, 5,780,588, and 6,214,345, and International Patent Application Publication Nos. WO02088172, WO2004010957, WO2005081711, WO2005084390, WO2006132670, WO03026577, WO200700860, WO207011968 and WO205082023.

In some embodiments, the antibody is conjugated to a pyrrolo [2,1-c][1,4]-benzodiazepine (PDB) or a peptide analog, derivative, or prodrug thereof. Suitable PDB and PDB derivatives and related art are described, for example, in Hartley J. A. et al., Cancer Res 2010; 70(17):6849-6858; Antonow D. et al., Cancer J 2008; 14(3):154-169; Howard P. W. et al., Bioorg Med Chem Lett 2009; 19:6463-6466 and Sagnou et al., Bioorg Med Chem Lett 2000; 10(18):2083-2086.

In some embodiments, the antibody is conjugated to a cytotoxic moiety selected from anthracyclines, maytansines, calicheamicins, duocarmycins, rachelmycin (CC-1065), dolastatin 10, dolastatin 15, irinotecan, monomethyl auristatin E, monomethyl auristatin F, PDB, or any analog, derivative or prodrug thereof.

In some embodiments, the antibody is conjugated to anthracycline or an analog, derivative, or prodrug thereof. In some embodiments, the antibody is conjugated to maytansine or an analog, derivative, or prodrug thereof. In some embodiments, the antibody is conjugated to a calicheamicin or an analog, derivative, or prodrug thereof. In some embodiments, the antibody is conjugated to a duocarmycin or an analog, derivative, or prodrug thereof. In some embodiments, the antibody is conjugated to rachelmycin (CC-1065) or an analog, derivative, or prodrug thereof. In some embodiments, the antibody is conjugated to dolastatin 10 or an analog, derivative, or prodrug thereof.

In some embodiments, the antibody is conjugated to dolastatin 15 or an analog, derivative, or prodrug thereof. In some embodiments, the antibody is conjugated to monomethyl auristatin E or an analog, derivative, or prodrug thereof. In some embodiments, the antibody is conjugated to monomethyl auristatin F or an analog, derivative, or prodrug thereof. In some embodiments, the antibody is conjugated to a pyrrolo [2,1-c][1,4]-benzodiazepine or an analog, derivative, or prodrug thereof. In some embodiments, the antibody is conjugated to irinotecan or an analog, derivative, or prodrug thereof.

In some embodiments, the antibody is conjugated to a cytokine (e.g. IL-2, IL-4, IL-6, IL-7, IL-10, IL-12, IL-13, IL-15, IL-18, IL-23, IL-24, IL-27, IL-28a, IL-28b, IL-29, KGF, IFNα, IFNβ, IFNγ, GM-CSF, CD40L, Flt3 ligand, stem cell factor, ancestim, and TNFα).

In some embodiments, the antibody is conjugated to a radioisotope or a chelate containing a radioisotope. For example, the antibody can be conjugated to a chelator linker (e.g. DOTA, DTPA, or thirace) that allows the antibody to complex with the radioisotope. The antibody may also or alternatively comprise or be conjugated to one or more radiolabeled amino acids or other radiolabeled molecules. Non-limiting examples of radioisotopes include ³H, ¹⁴C, ¹⁵N, ³⁵S, ⁹⁰Y, ⁹⁹Tc, ¹²⁵I, ¹³¹I, ¹⁸⁶Re, ²¹³Bi, ²²⁵Ac and ²²⁷Th. For therapeutic purposes, radioisotopes emitting β or α particle radiation may be used, such as ¹³¹I, ⁹⁰Y, ²¹¹At, ²¹²Bi, ⁶⁷Cu, ¹⁸⁶Re, ¹⁸⁸Re, and ²¹²Pb.

Techniques for coupling molecules to antibodies are well-known in the art. Typically, the nucleic acid molecule is covalently linked to a lysine or cysteine on the antibody via an N-hydroxysuccinimide ester or maleimide functional group, respectively. Conjugation methods using engineered cysteines or incorporating unnatural amino acids have been reported to improve the homogeneity of conjugates. In particular, those skilled in the art may also predict that reactive endogenous glutamine-engineered Fc-containing polypeptides are generated by use of acyl donor glutamine-containing tags (e.g. Gin peptide-containing tags or Q-tags) or by polypeptide engineering (e.g. by amino acid deletions, insertions, substitutions, or mutations in the polypeptide). Transglutaminase can then be covalently crosslinked with amine donors (e.g., small molecules containing or linked to reactive amines) to form stable and homogeneous groups of engineered polypeptides containing Fc, wherein amine donors are specifically coupled to polypeptides containing Fc through glutamine tags containing acyl donor or accessible/exposure/reactive endogenous glutamine sites (WO2012059882).

It should be understood that the therapeutic agents according to the above embodiments will be administered together with suitable pharmaceutically acceptable carriers, excipients, and other reagents incorporated into the formulation to provide improved transfer, delivery, tolerability, etc. A large number of suitable formulations can be found in all pharmacopoeias known to pharmaceutical chemists: Remington's Pharmaceutical Sciences (15^{th} edition, Mack Publishing Company, Easton, Pa. (1975)), in particular Chapter 87 of Blaug, Seymour therein. These formulations include, for example, powders, pastes, ointments, gels, waxes, oils, lipids, lipid-containing (cationic or anionic) carriers (e.g. Lipofectin™), DNA conjugates, anhydrous syrups, oil-in-water and water-in-oil emulsions, emulsion polyethylene glycols (polyethylene glycols of various molecular weights), semi-solid gels, and semi-solid mixtures containing polyethylene glycol.

Any of the foregoing mixtures may be suitable for treatments or therapies of the present disclosure, provided that the active ingredients in the formulation are not inactivated by the formulation and that the formulation is physiologically compatible and tolerates the routes of administration.

In one embodiment, the antibody can be used as a therapeutic agent. Such reagents will generally be used to treat, alleviate and/or prevent a disease or pathology associated with aberrant BCMA expression, activity, and/or signaling in a subject. Treatment regimens can be performed using standard methods by identifying a subject, e.g. a human patient, having (or at risk of or developing) a disease or disorder associated with aberrant BCMA expression, activity, and/or signaling, e.g. a BCMA-related disorder. An antibody preparation, preferably one preparation with high specificity and affinity for its target antigen, is administered to a subject and will generally have an effect due to its binding to the target. The administered antibody can eliminate or inhibit or interfere with the expression, activity, and/or signaling function of the target (e.g. BCMA). The administered antibody can eliminate or inhibit or interfere with the binding of the target (e.g. BCMA) to the endogenous ligand to which it naturally binds. For example, an antibody binds to a target, and modulates, blocks, inhibits, reduces, antagonizes, neutralizes, and/or otherwise interferes with BCMA expression, activity, and/or signaling. In some embodiments, in order to treat a disease or disorder associated with aberrant BCMA expression, an antibody having a heavy chain and light chain CDR can be administered to a subject.

In another embodiment, antibodies directed against BCMA can be used in methods known in the art and related to localization and/or quantitation of BCMA (e.g. for determining BCMA and/or levels of BCMA in an appropriate physiological sample, for diagnostic methods, for protein imaging, etc.).

In a given embodiment, an antibody comprising an antibody-derived antigen-binding domain specific for BCMA, or a derivative, fragment, analog, or homolog thereof, is used as a pharmaceutically active compound (hereinafter “therapeutic agent”).

In another embodiment, BCMA polypeptides can be isolated using antibodies specific for BCMA by standard techniques such as immunoaffinity, chromatography, or immunoprecipitation.

Antibodies (or fragments thereof) directed against BCMA proteins can be used to detect proteins in biological samples. In some embodiments, BCMA can be detected in a biological sample as part of a clinical testing procedure, for example, to determine the efficacy of a given treatment regimen.

Detection may be facilitated by coupling (i.e. physically linking) the antibody to a detectable substance. Examples of detectable substances include various enzymes, prosthetic groups, fluorescent materials, luminescent materials, bioluminescent materials, and radioactive materials.

Examples of suitable enzymes include horseradish peroxidase, alkaline phosphatase, β-galactosidase or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazine aminofluorescein, dansyl chloride, or phycoerythrin; one example of a luminescent material includes luminol; examples of bioluminescent materials include luciferase, luciferin, and aequorin, and examples of suitable radioactive materials include ¹²⁵I, ¹³¹I, ³⁵S or ³H.

In another embodiment, antibodies according to the present disclosure can be used as reagents for detecting the presence of BCMA or protein fragments thereof in a sample. In some embodiments, the antibody comprises a detectable label. The antibody is a polyclonal antibody, or more preferably a monoclonal antibody. Whole antibodies or fragments thereof (e.g. Fab, scFv, or F(ab′)₂) are used.

The term “labeling” with respect to an antibody is intended to encompass direct labeling of the antibody by coupling (i.e. physically linking) a detectable substance to the antibody, as well as indirect labeling of the antibody by reaction with another reagent that is directly labeled. Examples of indirect labeling include the detection of the first antibody using a fluorescently labeled second antibody, and end-labeling of the antibody with biotin so as to enable detection with fluorescently labeled streptavidin. The term “biological sample” is intended to include tissues, cells, and biological fluids isolated from a subject, as well as tissues, cells, and fluids present in a subject.

Thus, the term “biological sample” as used includes blood and fractions or components of blood, including serum, plasma, or lymph. In other words, the detection method of the above embodiments can be used to detect the analyte mRNA, protein, or genomic DNA in a biological sample in vitro as well as in vivo. For example, analyte mRNA in vitro detection techniques include Norhtern hybridization and in situ hybridization. In vitro techniques for the detection of analyte proteins include enzyme-linked immunosorbent assays (ELISA), western blots, immunoprecipitations, and immunofluorescence. Analyte genomic DNA in vitro detection techniques including Southern hybridization. Procedures for performing immunoassays are described, for example, in “ELISA: Theory and Practice: Methods in Molecular Biology”, vol. 42, J. R. Crowther (ed.) Human Press, Totowa, N. J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. In addition, in vivo techniques for the detection of an analyte protein include introducing into a subject a labeled anti-analyte protein antibody. For example, an antibody can be labeled with a radiolabel, and the presence and location of the radiolabel in the subject can then be detected by standard imaging techniques.

The antibodies and derivatives, fragments, analogs, and homologs thereof described herein can be incorporated into pharmaceutical compositions suitable for administration. The principles and considerations involved in preparing such compositions, as well as guidance in selecting components, are well known in the art, for example, see Remington's Pharmaceutical Sciences: The Science And Practice Of Pharmacy 19^{th} edition (Alfonso R. Gennaro et al. eds.) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, vol. 4), 1991, M. Dekker, New York.

Such compositions typically comprise an antibody and a pharmaceutically acceptable carrier. When antibody fragments are used, minimal inhibitory fragments that specifically bind to the target protein binding domain may be preferred. For example, based on the variable region sequence of an antibody, peptide molecules can be designed so as to retain the ability to bind to a target protein sequence. Such peptides can be chemically synthesized and/or produced by recombinant DNA techniques (see, e.g. Marasco et al. Proc. Natl. Acad. Sci. USA, 90: 7889-7893 (1993)).

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable pharmaceutically acceptable carriers are described in the latest edition of Remington's Pharmaceutical Sciences, a standard reference text in the art, which is incorporated herein by reference. Preferred examples of such carriers or diluents include, but are not limited to, water, saline, ringer's solution, dextrose solution, and 5% human serum albumin. Liposomes and non-aqueous vehicles such as fixed oils can also be used. It is well-known in the art that such media and reagents are used as pharmaceutically active substances. Except insofar as any conventional media or reagent is incompatible with the antibody, its use in the compositions is contemplated.

The pharmaceutical compositions of the above embodiments are formulated to be compatible with their intended routes of administration. Examples of routes of administration include parenteral, e.g. intravenous, intradermal, subcutaneous, oral (e.g. inhalation), transdermal (i.e. topical), transmucosal, and rectal administration. Solutions or suspensions for parenteral, intradermal, or subcutaneous administration may include the following components: sterile diluents for injection such as water, saline solutions, fixed oils, polyethylene glycols, glycerol, propylene glycol or other synthetic solvents; antibacterial agents such as benzyl alcohol or methyl 4-hydroxybenzoate; antioxidants such as ascorbic acid or sodium bisulfite; chelators such as ethylene diamine tetraacetic acid (EDTA); buffers, such as acetates, citrates or phosphates, and reagents to adjust the osmotic pressure, such as sodium chloride or dextrose. The pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injection use include sterile aqueous solutions (where water is soluble) or dispersions and sterile powders for the extemporaneous preparation of sterile injectable solutions or dispersion. For intravenous administration, suitable pharmaceutically acceptable carriers include physiological saline, bacteriostatic water, Cremophor EL™ (BASF, Parsippany, N.J.), or phosphate-buffered saline (PBS). In all cases, the composition must be sterile and should have a fluidity to the extent that is easy for injection. It must be stable under the conditions of manufacture and storage, and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), and suitable mixtures thereof. The proper fluidity can be maintained, for example, by use of a coating such as lecithin, by the maintenance of the required particle size in the case of dispersion, and by use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyols (such as mannitol, and sorbitol), and sodium chloride in the composition. Prolonged absorption of injectable compositions can be brought about by including in the composition a reagent that delays absorption, for example, aluminum monostearate and gelatin.

If needed, sterile injectable solutions can be prepared by incorporating the antibody in a required amount in an appropriate solvent with one or a combination of ingredients enumerated above (as required) followed by filtered sterilization. Generally, dispersions are prepared by incorporating the antibody into a sterile vehicle that contains a basic dispersion medium and the required other ingredients from those enumerated above. In the case of sterile powders for the preparation of sterile injectable solutions, preparation methods are vacuum drying and freeze-drying that obtain a powder, the powder includes the active ingredient and any additional desired ingredient from a previously sterile-filtered solution of such ingredients.

For inhaled administration, the compound is delivered in the form of an aerosol spray from a pressurized container or distributor or sprayer containing suitable propellants such as carbon dioxide. Systemic administration may also be performed by transmucosal or transdermal ways. For transmucosal or transdermal administration, a penetrant suitable for a permeation barrier should be used in the preparation. Such penetrants are generally known in the art, and include, for example, transmucosal administration, detergents, bile salts, and fusidic acid derivatives. Transmucosal administration can be accomplished by use of nasal sprays or suppositories. For transdermal administration, one or more of the antibodies can be formulated into ointments, salves, gels, or creams as generally known in the art.

The compounds may also be prepared in a form of suppositories (e.g. with conventional suppository bases such as cocoa butter or other glycerides) or retention enemas for rectal delivery.

In one embodiment, the antibodies can be prepared with carriers that prevent their rapid elimination from the body, such as sustained/controlled release formulations, including implants and microencapsulated delivery systems. Biodegradable, biocompatible polymers can be used, such as ethylene-vinyl acetate, polyanhydrides, polyglycolic acid, collagen, polyorthoesters, and polylactic acid. Methods for preparing such formulations will be apparent to those skilled in the art.

It is especially advantageous to formulate parenteral compositions in dosage unit form for ease of administration and uniformity of dosage. The dosage unit form, as used herein, refers to physically discrete units suited as unitary dosages for the subject to be treated; each unit contains a predetermined quantity of one or more antibodies calculated to produce the desired therapeutic effect in association with the required pharmaceutical carrier. The specification for the dosage unit forms of the above embodiments is dictated by and directly dependent on the unique characteristics of antibodies and the specific therapeutic effects to be achieved, and the limitations inherent in the art of formulating such antibodies for treating individuals.

The pharmaceutical compositions can be presented in a container, pack, or dispenser together with instructions for administration.

The formulations described herein may also contain more than one antibody depending on the particular situation to be treated, preferably those which have complementary activities but do not adversely affect each other. Alternatively, or additionally, the composition may, for example, comprise a reagent that enhances its function, such as a cytotoxic reagent, a cytokine, a chemotherapeutic agent, or a growth inhibitor. Such molecules are suitably present in combination in amounts effective for the intended purpose. For example, they may also be present in combination in a kit or in combination for use.

In one embodiment, one or more antibodies can be administered in a combination therapy, i.e. in combination with other reagents such as therapeutic agents that can be used to treat pathological conditions or disorders, such as various forms of cancer, autoimmune disorders, and inflammatory diseases. The term “in combination” means herein that the reagents are administered substantially synchronously, simultaneously, or sequentially. If administered sequentially, the first of the two compounds is still preferably detected at an effective concentration at the treatment site when the second compound is initially administered. In one instance, “in combination” can also include both an antibody of the present disclosure and another therapeutic agent in a kit.

For example, a combination therapy can comprise coformulation and/or coadministration of one or more antibodies described herein with one or more additional therapeutic agents (e.g. one or more cytokine and growth factor inhibitors, immunosuppressants, anti-inflammatory agents, metabolic inhibitors, enzyme inhibitors, and/or cytotoxic or cytostatic agents, as described in more detail below). Such combination therapy may advantageously utilize lower dosages of the administered therapeutic agents, thus avoiding possible toxicities or complications associated with each monotherapy.

In one embodiment, the administration of the anti-BCMA antibody and the administration of the therapeutic agent can reduce the number of B cells in the subject.

In one embodiment, the treatment regimen is effective to reduce cytokine release associated with administration of the T cell activating therapeutic agent in the subject, as compared to a corresponding treatment regimen without administration of an anti-BCMA antibody.

For purposes of clarity and conciseness, features are described herein as part of the same or separate embodiments, however, it will be understood that the scope of the disclosure may include some embodiments having a combination of all or some of the features described.

### EXAMPLES

### Example 1. Antigen Expression and Construction of Stably Transfected Cells

Using human BCMA (Sino biological, HG10620-M) and rhesus monkey BCMA (Sino biological, CG90103-G, identical with the extracellular region sequence of cynomolgus monkey XP_005591343.1) as templates, respectively, human BCMA (Met1-Asn53) or rhesus monkey BCMA (Met1-Asn52) were amplified, fused with mouse IgG2a Fc at C-terminal, and transfected into HEK293E cells to express the bivalent recombinant protein of human or cynomolgus monkey BCMA-mFc (the amino acid sequence shown in SEQ ID NOs. 1-2); or, fused with human IgG Fc (knob), and co-transfected into HEK293E cells with human IgG Fc (hole) by using knob-into-hole technology, the human or cynomolgus monkey BCMA-hFc kih monovalent recombinant protein (amino acid sequence shown in SEQ ID NOs. 3-5) was obtained. FIG. 1 shows SDS-PAGE results for human, cynomolgus monkey BCMA-mFc bivalent recombinant protein and BCMA-hFc kih monovalent recombinant protein.

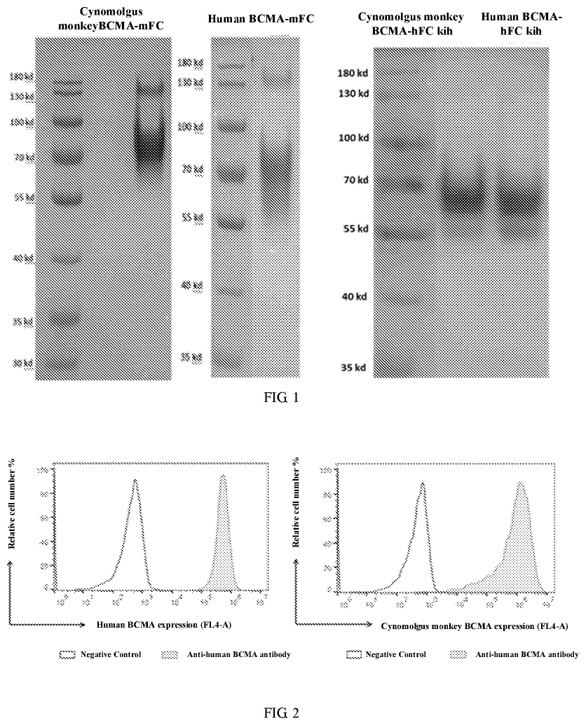

The Lenti vector and packaging plasmid (Genecoepia) containing the full-length sequences of human and cynomolgus monkey BCMA were co-transfected into HEK293T cells. 48 h after transfection, the culture supernatant containing pseudoviruses was collected. 10 μL was used to infect 1×10⁶ CHO cells, and different concentrations of puromycin were added to screen for resistance. Finally, stably transfected cells CHO-hBCMA (clone 2C2) and CHO-cynoBCMA (clone 1A2) highly expressing human or cynomolgus monkey BCMA were isolated. FIG. 2 shows that both human and monkey BCMA stably transfected cells detected by flow cytometry, both human BCMA stably transfected cells (CHO-hBCMA-2C2) and cynomolgus monkey BCMA stably transfected cells (CHO-cynoBCMA-1A2) express high levels of BCMA.

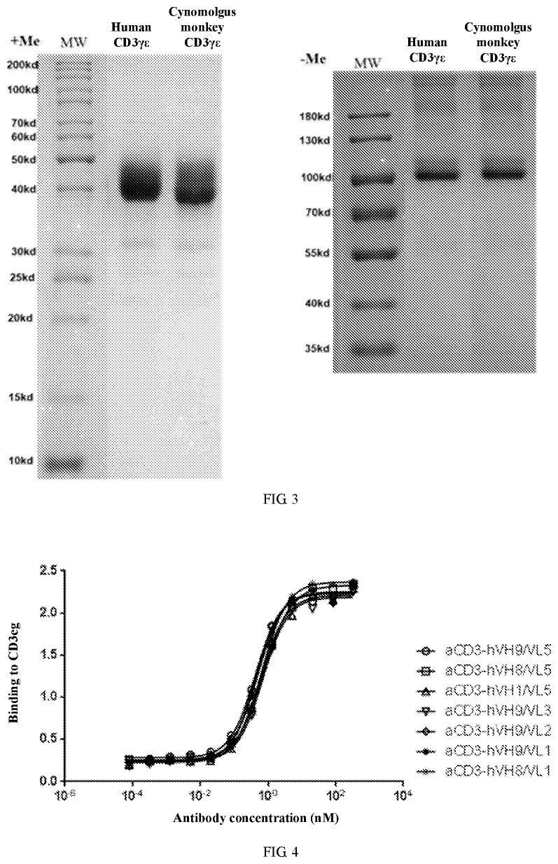

Construction of human or cynomolgus monkey CD3εγ heterodimers: the nucleotide sequences of the extracellular regions of human CD3γ (UniProt P09693, Gln23-Asn116) and CD3ε (UniProt P07766, Gln23-Asp126) are synthesized, fused with human IgG Fc hole or Fc knob respectively at the C-terminus, and to expressed to form a human CD3εγ-Fc heterodimer (the amino acid sequence of human CD3γ IgG Fc (hole) is shown in SEQ ID NO. 6, and the amino acid sequence of human CD3ε IgG Fc (knob) is shown in SEQ ID NO. 7); cynomolgus monkey CD3γ (UniProt Q95LI7, Gln23-Asn110) and CD3e (UniProt Q95LI5, Gln22-Asp117) were also synthesized, C-terminus fused to cynomolgus monkey IgG Fc hole or Fc knob and expressed to form a cynomolgus monkey CD3εγ-Fc heterodimer (the amino acid sequence of cynomolgus monkey CD3γ IgG Fc (hole) is shown in SEQ ID NO. 8 and the amino acid sequence of cynomolgus monkey CD3γ IgG Fc (knob) is shown in SEQ ID NO. 9). Recombinant plasmids expressing CD3γ-Fc and CD3ε-Fc were mixed with 3 mg/mL PEI (Polysciences, #24765-2) and co-transfected into HEK293E cells (culture medium OPM-293 CD03 DPM). After 7 days at 37° C. under 120 rpm 5% CO₂, the culture supernatant was collected and purified by Protein A affinity chromatography to obtain human or cynomolgus monkey CD3εγ-Fc recombinant protein with an SDS-PAGE purity higher than 95% (FIG. 3).

### Example 2. Animal Immunization and Preparation of BCMA Antibodies

Six-week-old female Balb/c mice (Charles River Beijing) were immunized with BCMA-mFc recombinant protein or CHO-BCMA stably transfected cells as immunogens, mixed with an equal volume of immune adjuvant (Titermax, Sigma-Aldrich), respectively, by footpad or subcutaneous immunization. After the primary immunization, the same dose of booster immunization is given every two weeks, and antigen immunization of human and monkey is alternated. After four immunizations, BCMA antibody titers in mouse serum greater than 1:10000 were detected. Three days after tail vein rush immunization, the spleen and part of the peripheral lymph nodes of mice were harvested, and total RNA was extracted. Antibody cDNA was obtained by reverse transcription with IgG-specific primers. The light and heavy chain variable region fragments were amplified respectively, and the phage Fab library was constructed. The number of transformed colonies (pool capacity) was about 1.2×10¹⁰.

### 1. Screening of Phage Antibody Libraries

Using classical panning, after mixing 1 mL BCMA immune library phage with 3.5 mL of 2% BSA/PBS solution, Maxisorp Immunotube (Nunc) coated with human or cynomolgus monkey BCMA recombinant protein was added. After binding for 1 h, multiple washing cycles were performed with PBS-Tween (0.5% v/v) and PBS to remove non-specific or low affinity bound phage. High affinity bound phage was eluted with 100 mM triethylamine and neutralized to infect TG1 cells. After two rounds of screening, a single clone was picked and inoculated into a 96-well U-shaped plate, 1 mM IPTG was added to induce expression, the induced supernatant containing Fab was used for ELISA detection screening, and 265 positive clones were obtained through primary screening.

After re-induction of expression, 10 μg/ml of biotinylated, labeled human BCMA recombinant protein was immobilized on an SA probe for 200 s of binding time and 300 s of dissociation time and tested for affinity to Fab (Octet, Fortebio). Fifty-nine clones with higher affinity were selected to detect the binding to cell membrane surface receptors using human and cynomolgus monkey BCMA-CHO stably transfected cells, wherein, fifty-two clones could cross-recognize human and cynomolgus monkey BCMA-CHO stably transfected cells.

2. Antibody Affinity Assay with Biacore

Partial high-affinity antibody clones were selected, plasmids were extracted and transformed into TG1 cells, expression was induced overnight at 0.1 mM IPTG 30° C., recombinantly expressed Fab was extracted from a bacterial periplasmic cavity, and Fab antibody was purified by Ni-NTA affinity chromatography. The Biacore affinity assay utilized a Protein A chip (GE healthcare), which was set to capture human BCMA-hFc recombinant protein (0.5 μg/mL) at 200KRU and after the baseline was stable, a gradient dilution of antibody Fab (from 10 μg/mL, 2-fold dilutions for 7 gradients) was run through the chip at a flow rate of 30 μL/min for a binding time of 350 s and a dissociation time of 600 s. Kinetic constants were fitted using Biacore T200 evaluation software with a Langmuir 1:1 kinetics model. The results of the BCMA antibody affinity assay are shown in Table 1.

TABLE 1
The affinity of BCMA Antibodies

| Fab | ka (1/Ms) | kd (1/s) | KD (nM) |
|---|---|---|---|
| 3G11 | 3.33E+04 | 4.32E−04 | 13.0 |
| 6G5 | 2.94E+04 | 6.13E−05 | 2.1 |
| 15G9 | 1.03E+06 | 1.26E−02 | 12.2 |
| 15H10 | 3.78E+04 | 6.90E−04 | 18.3 |
| 16B9 | 1.17E+05 | 4.76E−04 | 4.1 |
| 23F8 | 5.77E+04 | 3.22E−04 | 5.6 |
| 23G9 | 4.07E+03 | 3.13E−04 | 76.9 |
| 29A11 | 7.64E+04 | 2.43E−04 | 3.2 |
| 34G2 | 1.42E+05 | 7.05E−04 | 5.0 |
| 38E2 | 9.88E+04 | 7.98E−04 | 8.1 |
| 38D9 | 5.91E+04 | 3.29E−04 | 5.6 |
| 38F9 | 5.81E+03 | 2.00E−03 | 344 |
| 40C6 | 4.21E+04 | 2.71E−04 | 6.4 |

The BCMA antibody variable region sequences are as follows:

TABLE 2
Variable region sequences of BCMA murine antibody

| | HCVR | | HCDR1 | HCDR2 | HCDR3 | LCVR | | LCDR1 | LCDR2 | LCDR3 |
|---|---|---|---|---|---|---|---|---|---|---|
| BCMA | Amino | | Amino | Amino | Amino | Amino | | Amino | Amino | Amino |
| murine | acid | Nucleotide | acid | acid | acid | acid | Nucleotide | acid | acid | acid |
| antibody | sequence | sequence | Sequence | Sequence | Sequence | sequence | sequence | Sequence | Sequence | Sequence |
| 3G11 | 10 | 11 | 12 | 13 | 14 | 15 | 16 | 17 | 18 | 19 |
| 6G5 | 10 | 11 | 12 | 13 | 14 | 20 | 21 | 22 | 23 | 19 |
| 15G9 | 24 | 25 | 26 | 27 | 28 | 29 | 30 | 31 | 32 | 33 |
| 15H10 | 34 | 35 | 12 | 36 | 37 | 38 | 39 | 17 | 40 | 19 |
| 16B9 | 41 | 42 | 12 | 43 | 44 | 45 | 46 | 47 | 18 | 19 |
| 23F8 | 48 | 49 | 12 | 50 | 14 | 51 | 52 | 17 | 40 | 19 |
| 23G9 | 53 | 54 | 55 | 56 | 57 | 58 | 59 | 60 | 61 | 62 |
| 29A11 | 10 | 11 | 12 | 13 | 14 | 38 | 39 | 17 | 40 | 19 |
| 34G2 | 63 | 64 | 65 | 66 | 14 | 67 | 68 | 17 | 40 | 19 |
| 38E2 | 69 | 70 | 71 | 72 | 73 | 74 | 75 | 76 | 77 | 78 |
| 38D9 | 79 | 80 | 12 | 13 | 14 | 81 | 82 | 83 | 84 | 19 |
| 38F9 | 10 | 11 | 12 | 13 | 14 | 85 | 86 | 87 | 88 | 89 |
| 40C6 | 10 | 11 | 12 | 13 | 14 | 90 | 91 | 92 | 23 | 19 |

3G11

The amino acid sequence of the 3011 heavy chain VH is shown in SEQ ID NO. 10, of which the encoding nucleic acid is shown in SEQ ID NO. 11, and the HCDR1, HCDR2, and HCDR3 are shown in SEQ ID NO. 12, 13 and 14, respectively.

<------------FR1------------> CDR1 <-----FR2---->
EIQLVQSGPELKKPGETVKISCKASGYIFTNFGMNWVRQA
CDR2 <-----------FR3----------->
PGKALKWMGWINTYTGEQIYADDFKGRFAFSLETSA
CDR3 <----FR4--->
STAYLQINNLKNEDTATYFCARGEIYYGYDVGFVYWGQGTLVTVSA
Nucleic acid sequence
GAGATCCAGTTGGTGCAGTCTGGACCTGAGCTGAAGAAGCCTGGAGAGAC
AGTCAAGATCTCCTGCAAGGCTTCTGGGTATATCTTCACAAACTTTGGAA
TGAACTGGGTGAGGCAGGCTCCAGGAAAGGCTTTAAAGTGGATGGGCTGG
ATAAACACCTACACTGGAGAACAAATATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGAGGGGAG
ATCTACTATGGTTACGACGTGGGCTTTGTTTACTGGGGCCAAGGGACTCT
GGTCACTGTCTCTGCA

The amino acid sequence of the 3G11 light chain VK is shown in SEQ ID NO. 15, of which the encoding nucleic acid is shown in SEQ ID NO. 16, and the HCDR1, HCDR2, and HCDR3 are shown in SEQ ID NO. 17, 18 and 19, respectively.

<---------FR1---------> CDR1 <-----FR2----->
DIVLTQSPASLTVSLGQRATISCRASKSVSTSGYSYMHWYQQ
CDR2 <----------FR3---------->
KPGQPPKLLIYLASDLESGVSARFSGSGSGTDFT
CDR3 <---FR4-->
LNIHPVEEEDAATYYCQHSRELPWTFGGGTKLEIK
Nucleic acid sequence
GATATTGTGCTAACTCAGTCTCCTGCTTCCTTAACTGTATCTCTGGGGCA
GAGGGCCACCATCTCATGCAGGGCCAGCAAAAGTGTCAGTACATCTGGCT
ATAGTTATATGCACTGGTACCAACAGAAACCAGGACAGCCACCCAAACTC
CTCATCTATCTTGCATCCGACCTAGAATCTGGGGTCTCTGCCAGGTTCAG
TGGCAGTGGGTCTGGGACAGACTTCACCCTCAACATCCATCCTGTGGAGG
AGGAGGATGCTGCAACCTATTACTGTCAACACAGTAGGGAACTTCCGTGG
ACGTTCGGTGGAGGCACCAAGCTGGAAATAAAA
6G5

The amino acid sequence of the 635 heavy chain VH is shown in SEQ ID NO. 10, of which the encoding nucleic acid is shown in SEQ ID NO. 11, and the HCDR1, HCDR2, and HCDR3 are shown in SEQ ID NO. 12, 13 and 14, respectively.

<------------FR1------------> CDR1 <-----FR2----->
EIQLVQSGPELKKPGETVKISCKASGYIFTNFGMNWVRQ
CDR2 <-----------FR3----------->
APGKALKWMGWINTYTGEQIYADDFKGRFAFSLETSA
CDR3 <----FR4--->
STAYLQINNLKNEDTATYFCARGEIYYGYDVGFVYWGQGTLVTVSA
Nucleic acid sequence
GAGATCCAGTTGGTGCAGTCTGGACCTGAGCTGAAGAAGCCTGGAGAGAC
AGTCAAGATCTCCTGCAAGGCTTCTGGGTATATCTTCACAAACTTTGGAA
TGAACTGGGTGAGGCAGGCTCCAGGAAAGGCTTTAAAGTGGATGGGCTGG
ATAAACACCTACACTGGAGAACAAATATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGAGGGGAG
ATCTACTATGGTTACGACGTGGGCTTTGTTTACTGGGGCCAAGGGACTCT
GGTCACTGTCTCTGCA

The amino acid sequence of the 6G5 light chain VK is shown in SEQ ID NO. 20, of which the encoding nucleic acid is shown in SEQ ID NO. 21, and the HCDR1, HCDR2, and HCDR3 are shown in SEQ ID NO. 22, 23 and 19, respectively.

<---------FR1---------> CDR1 <-----FR2----->
DIVLTQSPASLAISLGQRATISCRASKSVTTSGYSYIHWY
CDR2 <----------FR3---------->
QQKPGQPPKLLIYLASDLEAGVPARFSGSGSGTDFT
CDR3 <---FR4-->
LNIHPVEEEDAATYYCQHSRELPWTFGGGTKLELK
Nucleic acid sequence
GACATTGTGCTAACACAGTCTCCTGCTTCCTTAGCTATATCTCTGGGGCA
GAGGGCCACCATCTCATGCAGGGCCAGCAAAAGTGTCACTACATCTGGCT
ATAGTTATATACACTGGTACCAACAGAAACCAGGACAGCCACCCAAACTC
CTCATCTATCTTGCATCCGACCTAGAAGCTGGGGTCCCTGCCAGGTTCAG
TGGCAGTGGGTCTGGGACAGACTTCACCCTCAACATCCATCCTGTGGAGG
AAGAGGATGCTGCAACCTATTACTGTCAGCACAGTAGGGAGCTTCCGTGG
ACGTTCGGTGGAGGCACCAAGCTGGAGCTGAAA
15G9

The amino acid sequence of the 15G9 heavy chain VH is shown in SEQ ID NO. 24, of which the encoding nucleic acid is shown in SEQ ID NO. 25, and the HCDR1, HCDR2, and HCDR3 are shown in SEQ ID NO. 26, 27 and 28, respectively.

<------------FR1------------> CDR1 <-----FR2----->
QVQLQQSGPELVKPGASVNLSCKASGYTFTNYVIHWVKQ
CDR2 <-----------FR3----------->
KPGQGLEWIGYTIPYTDVTKYNEKFKAKATLTSDKSS
CDR3 <---FR4--->
STAFMELSSLTSEDSAVYYCARYDFDGYFDYWGQGTTLTVSS
Nucleic acid sequence
CAGGTCCAGCTGCAACAGTCTGGACCTGAGCTGGTGAAGCCTGGGGCTTC
AGTGAATTTGTCCTGCAAGGCTTCTGGATACACATTCACTAATTATGTTA
TACACTGGGTAAAGCAGAAGCCTGGGCAGGGCCTTGAGTGGATTGGATAT
ACTATCCCTTACACTGATGTTACTAAGTACAATGAGAAATTCAAAGCCAA
GGCCACACTGACTTCAGACAAATCCTCCAGCACAGCCTTCATGGAGCTCA
GCAGCCTGACCTCTGAGGACTCTGCGGTCTATTACTGTGCAAGATATGAT
TTCGACGGCTACTTTGACTACTGGGGCCAAGGCACCACTCTCACAGTCTC
CTCA

The amino acid sequence of the 15G9 light chain VK is shown in SEQ ID NO. 29, of which the encoding nucleic acid is shown in SEQ ID NO. 30, and the HCDR1, HCDR2, and HCDR3 are shown in SEQ ID NO. 31, 32 and 33, respectively.

<---------FR1---------> CDR1 <-----FR2----->
DIVMTQAAFSNPVTLGTSASISCRSSKSLLHSNGITYLYWY
CDR2 <-----------FR3----------->
LQKPGQSPQLLIYQMSNLASGVPDRFSSSGSGTDF
CDR3 <---FR4-->
TLRISRVEAEDVGVYYCAQNLELPWTFGGGTKLELK
Nucleic acid sequence
GATATTGTGATGACTCAGGCTGCATTCTCCAATCCAGTCACTCTTGGAAC
ATCAGCTTCCATCTCCTGCAGGTCTAGTAAGAGTCTCCTACATAGTAATG
GCATCACTTATTTGTATTGGTATCTGCAGAAGCCAGGCCAGTCTCCTCAG
CTCCTGATTTATCAGATGTCCAACCTTGCCTCAGGAGTCCCAGACAGGTT
CAGTAGCAGTGGGTCAGGAACTGATTTCACACTGAGAATCAGCAGAGTGG
AGGCTGAGGATGTGGGTGTTTATTACTGTGCTCAAAATCTAGAACTTCCG
TGGACGTTCGGTGGAGGCACCAAGCTGGAGCTGAAA
15H10

The amino acid sequence of the 15H10 heavy chain VH is shown in SEQ ID NO. 34, of which the encoding nucleic acid is shown in SEQ ID NO. 35, and the HCDR1, HCDR2, and HCDR3 are shown in SEQ ID NO. 12, 36 and 37, respectively.

<------------FR1------------> CDR1 <-----FR2----->
EVQLQQSGPELKKPGETVKISCKASGYTFTNFGMNWVRQTPG
CDR2 <-----------FR3----------->
KALKWMGWINTYTGDLIYGDDFKGRFAFSLETSA
CDR3 <---FR4--->
STAYLQINNLKNEDTATYFCARGEIYYGYDVGFAYWGQGTLVTVSA
Nucleic acid sequence
GAGGTCCAGCTGCAGCAGTCTGGACCTGAGCTGAAGAAGCCTGGAGAGAC
AGTCAAGATCTCCTGCAAGGCTTCTGGGTATACCTTCACAAACTTTGGAA
TGAACTGGGTGAGGCAGACTCCAGGAAAGGCTTTAAAGTGGATGGGCTGG
ATAAACACCTACACTGGAGACTTAATATATGGTGATGACTTTAAGGGACG
GTTTGCCTTCTCTTTGGAAACCTCTGCCAGCACTGCCTATTTACAGATCA
ACAACCTCAAAAATGAGGACACGGCTACATATTTCTGTGCAAGAGGGGAG
ATCTACTATGGTTACGACGTGGGGTTTGCTTACTGGGGCCAAGGGACTCT
GGTCACTGTCTCTGCA

The amino acid sequence of the 15H10 light chain VK is shown in SEQ ID NO. 38, of which the encoding nucleic acid is shown in SEQ ID NO. 39, and the HCDR1, HCDR2, and HCDR3 are shown in SEQ ID NO. 17, 40 and 19, respectively.

<---------FR1---------> CDR1 <-----FR2----->
DIVLTQSPASLAVSLGQRATISCRASKSVSTSGYSYMHWY
CDR2 <----------FR3---------->
QQKPGQPPKLLIYLASNLESGVPARFSGSGSGTDFT
CDR3 <---FR4-->
LNIHPVEEEDAATYYCQHSRELPWTFGGGTKLEIK
Nucleic acid sequence
GACATTGTGCTGACACAGTCTCCTGCTTCCTTAGCTGTATCTCTGGGGCA
GAGGGCCACCATCTCATGCAGGGCCAGCAAAAGTGTCAGTACATCTGGCT
ATAGTTATATGCACTGGTACCAACAGAAACCAGGACAGCCACCCAAACTC
CTCATCTATCTTGCATCCAACCTAGAATCTGGGGTCCCTGCCAGGTTCAG
TGGTAGTGGGTCTGGGACAGACTTCACCCTCAACATCCATCCTGTGGAGG
AGGAGGATGCTGCAACCTATTACTGTCAGCACAGTAGGGAGCTTCCGTGG
ACGTTCGGTGGAGGCACCAAGCTGGAAATCAAA
16B9

The amino acid sequence of the 16B9 heavy chain VH is shown in SEQ ID NO. 41, of which the encoding nucleic acid is shown in SEQ ID NO. 42, and the HCDR1, HCDR2, and HCDR3 are shown in SEQ ID NO. 12, 43 and 44, respectively.

<------------FR1------------> CDR1 <-----FR2----->
EIQLVQSGPELKKPGETVKISCKASGYIFTNFGMNWVR
CDR2 <-----------FR3----------->
QTPGKALKWMGWINTYTGETIYADDFKGLFAFSLEVPA
CDR3 <----FR4--->
STAYLQINNLKNEDTATYFCARGEIYYGFDVGFAYWGQGTLVTVSA
Nucleic acid sequence
GAGATCCAGTTGGTGCAGTCTGGACCTGAGCTGAAGAAGCCTGGAGAGAC
AGTCAAGATCTCCTGCAAGGCTTCTGGGTATATTTTCACAAACTTTGGAA
TGAACTGGGTGAGGCAGACTCCAGGAAAGGCTTTAAAGTGGATGGGCTGG
ATAAACACCTACACTGGAGAAACAATATATGCTGATGACTTCAAGGGACT
ATTTGCCTTCTCTTTGGAAGTGCCTGCCAGCACTGCCTATTTGCAGATCA
ACAACCTCAAAAATGAGGACACGGCTACATATTTCTGTGCAAGAGGGGAG
ATCTACTATGGTTTCGACGTGGGATTTGCTTACTGGGGCCAAGGGACTCT
GGTCACTGTCTCTGCA

The amino acid sequence of the 16B9 light chain VK is shown in SEQ ID NO. 45, of which the encoding nucleic acid is shown in SEQ ID NO. 46, and the HCDR1, HCDR2, and HCDR3 are shown in SEQ ID NO. 47, 18 and 19, respectively.

<---------FR1---------> CDR1 <-----FR2----->
DIVLTQSPASLTVSLGQRATISCRASKSVSTSGYSYIHWYQQ
CDR2 <----------FR3---------->
KPGQPPKLLIYLASDLESGVPARFSGSGSGTDFT
CDR3 <---FR4-->
LNIHPVEEEDAATYYCQHSRELPWTFGGGTKLELK
Nucleic acid sequence
GACATTGTGCTAACACAGTCTCCTGCTTCCTTAACTGTATCTCTGGGGCA
GAGGGCCACCATCTCATGCAGGGCCAGCAAAAGTGTCAGTACATCTGGCT
ATAGTTATATACACTGGTACCAACAGAAACCAGGACAGCCACCCAAACTC
CTCATCTATCTTGCATCCGACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAACACAGTAGGGAACTTCCGTGG
ACGTTCGGTGGAGGCACCAAGCTGGAGCTGAAA
23F8

The amino acid sequence of the 23F8 heavy chain VH is shown in SEQ ID NO. 48, of which the encoding nucleic acid is shown in SEQ ID NO. 49, and the HCDR1, HCDR2, and HCDR3 are shown in SEQ ID NO. 12, 50 and 14, respectively.

<------------FR1------------> CDR1 <-----FR2----->
EIQLVQSGPELKKPGETVKISCTASGYTFTNFGMNWVRQ
CDR2 <-----------FR3----------->
APGKALRWMGWINTYTGDQIYADDFKGRFAFSLETSA
CDR3 <----FR4--->
STAYLQINNLKNEDTATYFCARGEIYYGYDVGFVYWGQGTLVTVSA
Nucleic acid sequence
GAGATCCAGTTGGTGCAGTCTGGACCTGAGCTGAAGAAGCCTGGAGAGAC
AGTCAAGATCTCCTGCACGGCTTCTGGGTATACCTTCACAAACTTTGGAA
TGAACTGGGTGAGGCAGGCTCCAGGAAAGGCTTTAAGGTGGATGGGCTGG
ATAAACACCTACACTGGAGACCAAATATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGAGGGGAG
ATCTACTATGGTTACGACGTGGGCTTTGTTTATTGGGGCCAAGGGACTCT
GGTCACTGTCTCTGCA

The amino acid sequence of the 23F8 light chain VK is shown in SEQ ID NO. 51, of which the encoding nucleic acid is shown in SEQ ID NO. 52, and the HCDR1, HCDR2, and HCDR3 are shown in SEQ ID NO. 17, 40 and 19, respectively.

<---------FR1---------> CDR1 <-----FR2----->
DIVLTQSPASLAVSLGQRATISCRASKSVSTSGYSYMHWYQ
CDR2 <----------FR3---------->
QKPGQPPKLLIYLASNLESGVPARFSGSGSGTDFT
CDR3 <---FR4-->
LNIHPVEEEDAATYYCQHSRELPWTFGGGTKLEIK
Nucleic acid sequence
GACATTGTGCTAACACAGTCTCCTGCTTCCTTAGCTGTATCTCTGGGGCA
GAGGGCCACCATCTCATGCAGGGCCAGCAAAAGTGTCAGTACATCTGGCT
ATAGTTATATGCACTGGTACCAACAGAAACCAGGACAGCCACCCAAACTC
CTCATCTATCTTGCATCCAACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAGCACAGTAGGGAGCTTCCGTGG
ACGTTCGGTGGAGGCACCAAGCTGGAAATAAAA
23G9

The amino acid sequence of the 23G9 heavy chain VH is shown in SEQ ID NO. 53, of which the encoding nucleic acid is shown in SEQ ID NO. 54, and the HCDR1, HCDR2, and HCDR3 are shown in SEQ ID NO. 55, 56 and 57, respectively.

<------------FR1------------> CDR1 <-----FR2----->
EVQLQQSGTVLTRPGASVKLSCKASGYSFSSYWMHWLKQ
CDR2 <-----------FR3----------->
RPGQGLEWIGAIYPGNSRTTYNQKFKGEAKLTADTSA
CDR3 <---FR4--->
STAYMDLSSLTNADSAVYFCTREETYWGQGTLVTVSA
Nucleic acid sequence
GAGGTTCAGCTCCAGCAGTCTGGGACTGTGCTGACAAGGCCTGGGGCTTC
CGTGAAGTTGTCCTGCAAGGCTTCTGGCTACAGCTTTTCCAGCTACTGGA
TGCACTGGCTAAAACAGAGGCCTGGACAGGGTCTAGAATGGATTGGTGCT
ATTTATCCTGGAAATAGTCGTACTACCTACAACCAGAAGTTCAAGGGCGA
GGCCAAACTGACTGCGGACACTTCCGCCAGCACTGCCTACATGGACCTCA
GCAGCCTGACAAATGCCGACTCTGCGGTCTATTTCTGTACAAGAGAGGAG
ACTTACTGGGGCCAAGGGACTCTGGTCACTGTCTCTGCA

The amino acid sequence of the 23G9 light chain VK is shown in SEQ ID NO. 58, of which the encoding nucleic acid is shown in SEQ ID NO. 59, and the HCDR1, HCDR2, and HCDR3 are shown in SEQ ID NOs. 60, 61 and 62, respectively.

<---------FR1---------> CDR1 <-----FR2----->
DVVMTQTPLSLSVTIGQPASISCKSSQGLLDSYGVTYLNWL
CDR2 <----------FR3---------->
FQRPGQPPKRLIYLVSELDSGVPDRFTGSGSGTDF
CDR3 <---FR4-->
TLRISRVEAEDLGIYYCWQGSHFPLTFGAGTKLELK
Nucleic acid sequence
GATGTTGTGATGACCCAGACTCCACTCTCTTTGTCGGTTACCATTGGACA
ACCAGCCTCCATCTCTTGCAAGTCAAGTCAGGGCCTCTTAGATAGTTATG
GAGTGACATATTTGAATTGGTTGTTTCAGAGGCCAGGCCAGCCTCCAAAG
CGCCTAATCTATCTAGTGTCTGAACTGGACTCTGGAGTCCCTGACAGGTT
CACTGGCAGTGGATCAGGGACAGATTTCACACTGAGAATCAGCAGAGTGG
AGGCTGAGGATTTGGGAATTTATTATTGCTGGCAAGGTTCACATTTTCCT
CTCACGTTCGGTGCTGGGACCAAGCTGGAGCTGAAA
29A11

The amino acid sequence of the 29A11 heavy chain VH is shown in SEQ ID NO. 10, of which the encoding nucleic acid is shown in SEQ ID NO. 11, and the HCDR1, HCDR2, and HCDR3 are shown in SEQ ID NOs. 12, 13 and 14, respectively.

<------------FR1------------> CDR1 <-----FR2----->
EIQLVQSGPELKKPGETVKISCKASGYIFTNFGMNWVRQAPGKAL
CDR2 <-----------FR3----------->
KWMGWINTYTGEQIYADDFKGRFAFSLETSA
CDR3 <----FR4--->
STAYLQINNLKNEDTATYFCARGEIYYGYDVGFVYWGQGTLVTVSA
Nucleic acid sequence
GAGATCCAGCTGGTGCAGTCTGGACCTGAGCTGAAGAAGCCTGGAGAGAC
AGTCAAGATCTCCTGCAAGGCTTCTGGGTATATCTTCACAAACTTTGGAA
TGAACTGGGTGAGGCAGGCTCCAGGAAAGGCTTTAAAGTGGATGGGCTGG
ATAAACACCTACACTGGAGAACAAATATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGAGGGGAG
ATCTACTATGGTTACGACGTGGGCTTTGTTTACTGGGGCCAAGGGACTCT
GGTCACTGTCTCTGCA

The amino acid sequence of the 29A11 light chain VK is shown in SEQ ID NO. 38, of which the encoding nucleic acid is shown in SEQ ID NO. 39, and the HCDR1, HCDR2, and HCDR3 are shown in SEQ ID NOs. 17, 40 and 19, respectively.

<---------FR1---------> CDR1 <-----FR2----->
DIVLTQSPASLAVSLGQRATISCRASKSVSTSGYSYMHWY
CDR2 <----------FR3---------->
QQKPGQPPKLLIYLASNLESGVPARFSGSGSGTDFT
CDR3 <---FR4-->
LNIHPVEEEDAATYYCQHSRELPWTFGGGTKLEIK
Nucleic acid sequence
GATATTGTGCTAACTCAGTCTCCTGCTTCCTTAGCCGTATCTCTGGGGCA
GAGGGCCACCATCTCATGCAGGGCCAGCAAAAGTGTCAGTACATCTGGCT
ATAGTTATATGCACTGGTACCAACAGAAACCAGGACAGCCACCCAAACTC
CTCATCTATCTTGCATCCAACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAGCACAGTAGGGAGCTTCCGTGG
ACGTTCGGTGGAGGCACCAAGCTGGAAATCAAA
34G2

The amino acid sequence of the 34G2 heavy chain VH is shown in SEQ ID NO. 63, of which the encoding nucleic acid is shown in SEQ ID NO. 64, and the HCDR1, HCDR2, and HCDR3 are shown in SEQ ID NOs. 65, 66 and 14, respectively.

<------------FR1------------> CDR1 <-----FR2----->
EVQLQQSGPELRKPGETVKISCKASGYTFTNYGMNWVKQTPGKGL
CDR2 <-----------FR3----------->
KWMGWINTYTGERIYADDFKGRFAFSLETSA
CDR3 <----FR3--->
STAYLQINNLKNEDTATYFCARGEIYYGYDVGFVYWGQGTLVTVSA
Nucleic acid sequence
GAGGTCCAGTTGCAGCAGTCTGGACCTGAGCTGAGGAAGCCTGGAGAGAC
AGTCAAGATCTCCTGCAAGGCTTCTGGGTATACCTTCACAAACTATGGAA
TGAACTGGGTGAAGCAGACTCCAGGAAAGGGTTTAAAGTGGATGGGCTGG
ATAAACACCTACACTGGAGAACGAATATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGAGGGGAG
ATCTACTATGGTTACGACGTGGGCTTTGTTTACTGGGGCCAAGGGACTCT
GGTCACTGTCTCTGCA

The amino acid sequence of the 34G2 light chain VK is shown in SEQ ID NO. 67, of which the encoding nucleic acid is shown in SEQ ID NO. 68, and the HCDR1, HCDR2, and HCDR3 are shown in SEQ ID NOs. 17, 40 and 19, respectively.

<---------FR1---------> CDR1 <-----FR2----->
DIVLTQSPASLVVSLGQRATISCRASKSVSTSGYSYMHWYQ
CDR2 <----------FR3---------->
QKPGQPPKLLIYLASNLESGVPARFSGSGSGTDFT
CDR3 <---FR4-->
LNIHPVEEEDAATYYCQHSRELPWTFGGGTRLEIK
Nucleic acid sequence
GACATTGTGCTGACACAGTCTCCTGCTTCCTTAGTTGTATCTCTGGGGCA
GAGGGCCACCATCTCATGCAGGGCCAGCAAAAGTGTCAGTACATCTGGCT
ATAGTTATATGCACTGGTACCAACAGAAACCAGGACAGCCACCCAAACTC
CTCATCTATCTTGCATCCAACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAGCACAGTAGGGAGCTTCCGTGG
ACGTTCGGTGGAGGCACCAGACTGGAAATAAAA
38E2

The amino acid sequence of the 38E2 heavy chain VH is shown in SEQ ID NO. 69, of which the encoding nucleic acid is shown in SEQ ID NO. 70, and the HCDR1, HCDR2, and HCDR3 are shown in SEQ ID NOs. 71, 72 and 73, respectively.

<------------FR1------------> CDR1 <-----FR2----->
QVQLQQSGAELVRPGTSVKMSCKAAGYTFTNYWIGWVKQ
CDR2 <-----------FR3----------->
RPGHGLEWIGDIYPGSGDTNYNGKFKGKATLAADISS
CDR3 <----FR4--->
NAAYMQLSSLTSEDSAIYYCARSYYRNDAASFAYWGQGTLVTVSA
Nucleic acid sequence
CAGGTCCAGCTGCAGCAGTCTGGAGCTGAGCTGGTAAGGCCTGGGACTTC
AGTGAAGATGTCCTGCAAGGCTGCTGGATACACCTTCACTAACTACTGGA
TAGGTTGGGTAAAGCAGAGGCCTGGACATGGCCTTGAGTGGATTGGAGAT
ATTTACCCTGGAAGTGGTGATACTAATTACAATGGGAAGTTCAAGGGCAA
GGCCACACTGGCTGCAGACATTTCCTCCAACGCAGCCTACATGCAGCTCA
GCAGCCTGACATCTGAGGACTCTGCCATCTATTACTGTGCAAGAAGCTAC
TATAGGAACGACGCGGCCTCATTTGCTTACTGGGGCCAAGGGACTCTGGT
CACTGTCTCTGCA

The amino acid sequence of the 38E2 light chain VK is shown in SEQ ID NO. 74, of which the encoding nucleic acid is shown in SEQ ID NO. 75, and the HCDR1, HCDR2, and HCDR3 are shown in SEQ ID NOs. 76, 77 and 78, respectively.

<---------FR1---------> CDR1<-----FR2----->
DIVMTQSHKFMSTSVGDRVNITCKASQDVDTAVAWYQQKP
CDR2 <--------FR3-------->
SQSPKLLIYWTSTRHTGVPDRFTGSGSGTDFTLTIS
CDR3 <---FR4-->
NVQSEDLADYFCQQFSSYPLTFGAGTKLELK
Nucleic acid sequence
GACATTGTGATGACCCAGTCTCACAAATTCATGTCCACATCAGTAGGAGA
CAGGGTCAACATCACCTGCAAGGCCAGTCAGGATGTGGATACTGCTGTAG
CCTGGTATCAACAGAAACCAAGTCAATCTCCTAAACTACTGATTTACTGG
ACATCCACCCGGCACACTGGAGTCCCTGATCGCTTCACAGGCAGTGGATC
TGGGACAGATTTCACTCTCACCATTAGCAATGTGCAGTCTGAAGACTTGG
CAGATTATTTCTGTCAACAATTTAGTAGCTATCCTCTCACGTTCGGTGCT
GGGACCAAGCTGGAGCTGAAA
38D9

The amino acid sequence of the 38D9 heavy chain VH is shown in SEQ ID NO. 79, of which the encoding nucleic acid is shown in SEQ ID NO. 80, and the HCDR1, HCDR2, and HCDR3 are shown in SEQ ID NOs. 12, 13 and 14, respectively.

<------------FR1------------> CDR1 <-----FR2----->
EVQLQQSGPELKKPGETVKISCKASGYIFTNFGMNWVRQ
CDR2<-----------FR3----------->
APGKALKWMGWINTYTGEQIYADDFKGRFAFSLETSA
CDR3 <----FR4--->
STAYLQINNLKNEDTATYFCARGEIYYGYDVGFVYWGQGTLVTVSA
Nucleic acid sequence
GAGGTCCAGTTGCAGCAGTCTGGACCTGAGCTGAAGAAGCCTGGAGAGAC
AGTCAAGATCTCCTGCAAGGCTTCTGGGTATATCTTCACGAACTTTGGAA
TGAACTGGGTGAGGCAGGCTCCAGGAAAGGCTTTAAAGTGGATGGGCTGG
ATAAACACCTACACTGGAGAACAAATATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGAGGGGAG
ATCTACTATGGTTACGACGTGGGCTTTGTTTACTGGGGCCAAGGGACTCT
GGTCACTGTCTCTGCA

The amino acid sequence of the 38D9 light chain VK is shown in SEQ ID NO. 81, of which the encoding nucleic acid is shown in SEQ ID NO. 82, and the HCDR1, HCDR2, and HCDR3 are shown in SEQ ID NOs. 83, 84 and 19, respectively.

<---------FR1---------> CDR1 <-----FR2----->
DIVLTQSPASLTVSLGQRATISCGASKSVSTSGYSYMHWYQ
CDR2 <----------FR3---------->
QKPGQPPKLLIYLGSDLESGVPARFSGSGSGTDFT
CDR3 <---FR4-->
LNIHPVEEEDAATYYCQHSRELPWTFGGGTKLEIK
Nucleic acid sequence
GACATTGTGCTGACACAGTCTCCTGCTTCCTTAACTGTATCTCTGGGGCA
GAGGGCCACCATCTCATGCGGGGCCAGCAAAAGTGTCAGTACATCTGGCT
ATAGTTATATGCACTGGTACCAACAGAAACCAGGACAGCCACCCAAACTC
CTCATCTATCTTGGATCCGACCTAGAATCTGGGGTCCCTGCCAGGTTCAG
TGGCAGTGGGTCTGGGACAGACTTCACCCTCAACATCCATCCTGTGGAGG
AGGAGGATGCTGCAACCTATTACTGTCAACACAGTAGGGAACTTCCGTGG
ACGTTCGGTGGAGGCACCAAGCTGGAAATCAAA
38F9

The amino acid sequence of the 38F9 heavy chain VH is shown in SEQ ID NO. 10, of which the encoding nucleic acid is shown in SEQ ID NO. 11, and the HCDR1, HCDR2, and HCDR3 are shown in SEQ ID NOs. 12, 13 and 14, respectively.

<------------FR1------------> CDR1 <-----FR2---->
EIQLVQSGPELKKPGETVKISCKASGYIFTNFGMNWVRQ
CDR2 <-----------FR3----------->
APGKALKWMGWINTYTGEQIYADDFKGRFAFSLETSA
CDR3 <----FR4--->
STAYLQINNLKNEDTATYFCARGEIYYGYDVGFVYWGQGTLVTVSA
Nucleic acid sequence
GAGATCCAGTTGGTGCAGTCTGGACCTGAGCTGAAGAAGCCTGGAGAGAC
AGTCAAGATCTCCTGCAAGGCTTCTGGGTATATCTTCACAAACTTTGGAA
TGAACTGGGTGAGGCAGGCTCCAGGAAAGGCTTTAAAGTGGATGGGCTGG
ATAAACACCTACACTGGAGAACAAATATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGAGGGGAG
ATCTACTATGGTTACGACGTGGGCTTTGTTTACTGGGGCCAAGGGACTCT
GGTCACTGTCTCTGCA

The amino acid sequence of the 38F9 light chain VK is shown in SEQ ID NO. 85, of which the encoding nucleic acid is shown in SEQ ID NO. 86, and the HCDR1, HCDR2, and HCDR3 are shown in SEQ ID NOs. 87, 88 and 89, respectively.

<---------FR1---------> CDR1 <-----FR2----->
NIVLTQSPASLAVSLGQRATISCRASESVDSSGNSFMHWYQQ
CDR2 <------------FR3------------>
KPGQPPKLLIYLESNLESGVPARFSGSGSRTDFT
CDR3 <---FR4-->
LTIEPVEADDAATYYCQQSNEDPWTFGGGTKLEIK
Nucleic acid sequence
AACATTGTGCTGACCCAATCTCCAGCTTCTTTGGCTGTGTCTCTAGGGCA
GAGGGCCACCATATCCTGCAGAGCCAGTGAAAGTGTTGATAGTTCTGGCA
ATAGTTTTATGCACTGGTACCAGCAGAAACCAGGACAGCCACCCAAACTC
CTCATCTATCTTGAATCCAACCTAGAATCTGGGGTCCCTGCCAGGTTCAG
TGGCAGTGGGTCTAGGACAGACTTCACCCTCACCATTGAACCTGTGGAGG
CTGATGATGCTGCAACCTATTACTGTCAGCAAAGTAATGAGGATCCGTGG
ACGTTCGGTGGAGGCACCAAGCTGGAAATCAAA
40C6

The amino acid sequence of the 40C6 heavy chain VH is shown in SEQ ID NO. 10, of which the encoding nucleic acid is shown in SEQ ID NO. 11, and the HCDR1, HCDR2, and HCDR3 are shown in SEQ ID NOs. 12, 13 and 14, respectively.

<------------FR1------------> CDR1 <-----FR2----->
EIQLVQSGPELKKPGETVKISCKASGYIFTNFGMNWVRQ
CDR2 <-----------FR3----------->
APGKALKWMGWINTYTGEQIYADDFKGRFAFSLETSA
CDR3 <---FR4--->
STAYLQINNLKNEDTATYFCARGEIYYGYDVGFVYWGQGTLVTVSA
Nucleic acid sequence
GAGATCCAGTTGGTGCAGTCTGGACCTGAGCTGAAGAAGCCTGGAGAGAC
AGTCAAGATCTCCTGCAAGGCTTCTGGGTATATCTTCACAAACTTTGGAA
TGAACTGGGTGAGGCAGGCTCCAGGAAAGGCTTTAAAGTGGATGGGCTGG
ATAAACACCTACACTGGAGAACAAATATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGAGGGGAG
ATCTACTATGGTTACGACGTGGGCTTTGTTTACTGGGGCCAAGGGACTCT
GGTCACTGTCTCTGCA

The amino acid sequence of 40C6 light chain VK is shown in SEQ ID NO. 90, of which the encoding nucleic acid is shown in SEQ ID NO. 91, and the LCDR1, LCDR2, and LCDR3 are shown in SEQ ID NOs. 92, 23 and 19, respectively.

<---------FR1---------> CDR1 <-----FR2----->
DIVLTQSPASLAISLGQRATISCRASRSVTTSGYSYIHWYQQ
CDR2 <----------FR3---------->
KPGQPPKLLIYLASDLEAGVPARFSGSGSGTDFT
CDR3 <---FR4-->
LNIHPVEEEDAATYYCQHSRELPWTFGGGTKLEIK
Nucleic acid sequence
GACATTGTGCTGACACAGTCTCCTGCTTCCTTAGCTATATCTCTGGGGCA
GAGGGCCACCATCTCATGCAGGGCCAGCAGAAGTGTCACTACATCTGGCT
ATAGTTATATACACTGGTACCAACAGAAACCAGGACAGCCACCCAAACTC
CTCATCTATCTTGCATCCGACCTAGAAGCTGGGGTCCCTGCCAGGTTCAG
TGGCAGTGGGTCTGGGACAGACTTCACCCTCAACATCCATCCTGTGGAGG
AAGAGGATGCTGCAACCTATTACTGTCAGCACAGTAGGGAGCTTCCGTGG
ACGTTCGGTGGAGGCACCAAGCTGGAAATAAAA

### Example 3. Construction of BCMA×CD3 Bispecfc Antibody

### 1. CD3 Humanized Antibody

Murine hybridoma CD3 antibody (EMBO J. 1985.4(2): 337-344; J. Immunol. 1986,137(4): 1097-100; J. Exp. Med. 1991, 174: 319-326; J. Immunol. 1991, 147(9): 3047-52) recognizes the human and cynomolgus monkey CD3 receptor, the sequence of which is as follows:

the anti-CD3 murine monoclonal antibody light chain amino acid sequence is shown in SEQ ID NO. 184:

QAVVTQESALTTSPGETVTLTCRSSTGAVTTSNYANWVQ
QKPDHLFTGLIGGTNKRAPGVPARFSGSLIGDKAALTIT
GAQTEDEAIYFCALWYSNLWVFGGGTKLTVL

the amino acid sequence of the anti-CD3 murine monoclonal antibody heavy chain is shown in SEQ ID NO. 185:

EVQLVESGGGLVQPKGSLKLSCAASGFTFNTYAMNWVRQ
APGKGLEWVARIRSKYNNYATYYADSVKDRFTISRDDSQ
SILYLQMNNLKTEDTAMYYCVRHGNFGNSYVSWFAYWGQ
GTLVTVSS

the anti-CD3 murine monoclonal antibody was humanized, the human germline gene IMGT_hVL7-43 with the highest homology was selected for light chain CDR transplantation and human IGLJ3*02 was selected for FM4; human IMGT hVH3-73 was selected for heavy chain CDR transplantation and human IGHJ4*01 was selected for FM4. The different heavy chain and light chain variants were designed.

The sequence of the light chain variable region of the humanized CD3 antibody is as follows:

TABLE 3
Light chain variable region sequences
of CD3 humanized antibodies

| | SEQ ID NOs: 93-111 | | | | |
|---|---|---|---|---|---|
| | LCVR | | LCDR1 | LCDR2 | LCDR3 |
| CD3 | Amino | | Amino | Amino | Amino |
| humanized | acid | Nucleotide | acid | acid | acid |
| antibody | sequence | sequence | sequence | sequence | sequence |
| hVL1 | 93 | 94 | 95 | 96 | 97 |
| hVL2 | 98 | 99 | 95 | 96 | 97 |
| hVL3 | 100 | 101 | 102 | 103 | 97 |
| hVL4 | 104 | 105 | 102 | 103 | 97 |
| hVL5 | 106 | 107 | 95 | 96 | 108 |
| hVL6 | 109 | 110 | 95 | 111 | 108 |

The amino acid sequence of hVL1 is shown in SEQ ID NO. 93, of which the encoding nucleic acid is shown in SEQ ID NO. 94, and the LCDR1, LCDR2, and LCDR3 are shown in SEQ ID NOs. 95, 96 and 97, respectively.

QAVVTQEPSLTVSPGGTVTLTCRSSTGAVTTSNYANWVQ
QKPGQAPRGLIGGTNKRAPWTPARFSGSLLGGKAALTLS
GAQPEDEAEYYCALWYSNLWVFGGGTKLTVL

The amino acid sequence of hVL2 is shown in SEQ ID NO. 98, of which the encoding nucleic acid is shown in SEQ ID NO. 99, and the LCDR1, LCDR2, and LCDR3 are shown in SEQ ID NOs. 95, 96 and 97, respectively.

QAVVTQEPSLTVSPGGTVTLTCRSSTGAVTTSNYANWVQEKPGQAPRGLI
GGTNKRAPWTPARFSGSLLGGKAALTLSGAQPEDEAEYYCALWYSNLWVF
GGGTKLTVL

The amino acid sequence of hVL3 is shown in SEQ ID NO. 100, of which the encoding nucleic acid is shown in SEQ ID NO. 101, and the LCDR1, LCDR2, and LCDR3 are shown in SEQ ID NOs. 102, 103 and 97, respectively.

EAVVTQEPSLTVSPGGTVTLTCESSDGAVTTSNYANWVQEKPGQAPRGLI
GGTNKEAPWTPARFSGSLLGGKAALTLSGAQPEDEAEYYCALWYSNLWVF
GGGTKLTVL

The amino acid sequence of hVL4 is shown in SEQ ID NO. 104, of which the encoding nucleic acid is shown in SEQ ID NO. 105, and the LCDR1, LCDR2, and LCDR3 are shown in SEQ ID NOs. 102, 103 and 97, respectively.

QAVVTQEPSLTVSPGGTVTLTCESSDGAVTTSNYANWVQEKPGQAPRGLI
GGTNKEAPWTPARFSGSLLGGKAALTLSGAQPEDEAEYYCALWYSNLWVF
GGGTKLTVL

The amino acid sequence of hVL5 is shown in SEQ ID NO. 106, of which the encoding nucleic acid is shown in SEQ ID NO. 107, and the LCDR1, LCDR2, and LCDR3 are shown in SEQ ID NOs. 95, 96 and 108, respectively.

QAVVTQEPSLTVSPGGTVTLTCRSSTGAVTTSNYANWVQEKPGQAPRGLI
GGTNKRAPWTPARFSGSLLGGKAALTITGAQAEDEAEYYCVLWYSNLWVF
GGGTKLTVL

The amino acid sequence of hVL6 is shown in SEQ ID NO. 109, of which the encoding nucleic acid is shown in SEQ ID NO. 110, and the LCDR1, LCDR2, and LCDR3 are shown in SEQ ID NOs. 95, 111 and 108, respectively.

QAVVTQEPSLTVSPGGTVTLTCRSSTGAVTTSNYANWFQEKPGQAPRGLI
YGTNKRAPWTPARFSGSLLGGKAALTLSGAQAEDEAEYYCVLWYSNLWVF
GGGTKLTVL

The sequences of the heavy chain variable region of the humanized CD3 antibody is as follows:

TABLE 4
Heavy chain variable region sequences
of CD3 humanized antibodies

| | SEQ ID NOs: 112-141 | | | | |
|---|---|---|---|---|---|
| | HCVR | | HCDR1 | HCDR2 | HCDR3 |
| CD3 | Amino | | Amino | Amino | Amino |
| humanized | acid | Nucleotide | acid | acid | acid |
| antibody | sequence | sequence | Sequence | Sequence | Sequence |
| hVH1 | 112 | 113 | 114 | 115 | 116 |
| hVH2 | 117 | 118 | 119 | 115 | 116 |
| hVH3 | 120 | 121 | 119 | 115 | 122 |
| hVH4 | 123 | 124 | 119 | 115 | 125 |
| hVH5 | 126 | 127 | 119 | 115 | 128 |
| hVH6 | 129 | 130 | 119 | 115 | 131 |
| hVH7 | 132 | 133 | 134 | 135 | 116 |
| hVH8 | 136 | 137 | 114 | 115 | 116 |
| hVH9 | 138 | 139 | 114 | 115 | 116 |
| hVH10 | 140 | 141 | 114 | 115 | 116 |

The amino acid sequence of hVH1 is shown in SEQ ID NO. 112, of which the encoding nucleic acid is shown in SEQ ID NO. 113, and the HCDR1, HCDR2, and HCDR3 are shown in SEQ ID NOs. 114, 115 and 116, respectively.

EVQLVESGGGLVQPGGSLRLSCAASGFTFNTYAMNWVRQAPGKGLEWVSR
IRSKYNNYATYYADSVKDRFTISRDDSKNTLYLQMNSLKTEDTAVYYCVR
HGNFGNSYVSWFAYWGQGTLVTVSS

The amino acid sequence of hVH2 is shown in SEQ ID NO. 117, of which the encoding nucleic acid is shown in SEQ ID NO. 118, and the HCDR1, HCDR2, and HCDR3 are shown in SEQ ID NOs. 119, 115 and 116, respectively.

EVQLVESGGGLVQPGGSLRLSCAASGFTFSTYAMNWVRKAPGKGLEWVSR
IRSKYNNYATYYADSVKDRFTISRDDSKNTLYLQMNSLRAEDTAVYYCVR
HGNFGNSYVSWFAYWGQGTLVTVSS

The amino acid sequence of hVH3 is shown in SEQ ID NO. 120, of which the encoding nucleic acid is shown in SEQ ID NO. 121, and the HCDR1, HCDR2, and HCDR3 are shown in SEQ ID NOs. 119, 115 and 122, respectively.

EVQLVESGGGLVQPGGSLRLSCAASGFTFSTYAMNWVRKAPGKGLEWVSR
IRSKYNNYATYYADSVKDRFTISRDDSKNTLYLQMNSLRAEDTAVYYCVR
HGNFGESYVSWFAYWGQGTLVTVSS

The amino acid sequence of hVH4 is shown in SEQ ID NO. 123, of which the encoding nucleic acid is shown in SEQ ID NO. 124, and the HCDR1, HCDR2, and HCDR3 are shown in SEQ ID NOs. 119, 115 and 125, respectively.

EVQLVESGGGLVQPGGSLRLSCAASGFTFSTYAMNWVRKAPGKGLEWVSR
IRSKYNNYATYYADSVKDRFTISRDDSKNTLYLQMNSLRAEDTAVYYCVR
HGNFGQSYVSWFAYWGQGTLVTVSS

The amino acid sequence of hVH5 is shown in SEQ ID NO. 126, of which the encoding nucleic acid is shown in SEQ ID NO. 127, and the HCDR1, HCDR2, and HCDR3 are shown in SEQ ID NOs. 119, 115 and 128, respectively.

EVQLVESGGGLVQPGGSLRLSCAASGFTFSTYAMNWVRKAPGKGLEWVSR
IRSKYNNYATYYADSVKDRFTISRDDSKNTLYLQMNSLRAEDTAVYYCVR
HGNFGDSYVSWFAYWGQGTLVTVSS

The amino acid sequence of hVH6 is shown in SEQ ID NO. 129, of which the encoding nucleic acid is shown in SEQ ID NO. 130, and the HCDR1, HCDR2, and HCDR3 are shown in SEQ ID NOs. 119, 115 and 131, respectively.

EVQLVESGGGLVQPGGSLRLSCAASGFTFSTYAMNWVRKAPGKGLEWVSR
IRSKYNNYATYYADSVKDRFTISRDDSKNTLYLQMNSLRAEDTAVYYCVR
HGNFGTSYVSWFAYWGQGTLVTVSS

The amino acid sequence of hVH7 is shown in SEQ ID NO. 132, of which the encoding nucleic acid is shown in SEQ ID NO. 133, and the HCDR1, HCDR2, and HCDR3 are shown in SEQ ID NOs. 134, 135 and 116, respectively.

EVQLVESGGGLVQPGGSLRLSCAASGFTFSDYAMNWVRKAPGKGLEWVSR
IRSKYNNYATYYADSVEDRFTISRDDSKNTLYLQMNSLRAEDTAVYYCVR
HGNFGNSYVSWFAYWGQGTLVTVSS

The amino acid sequence of hVH8 is shown in SEQ ID NO. 136, of which the encoding nucleic acid is shown in SEQ ID NO. 137, and the HCDR1, HCDR2, and HCDR3 are shown in SEQ ID NOs. 114, 115 and 116, respectively.

EVQLVESGGGLVQPGGSLRLSCAASGFTFNTYAMNWVRKAPGKGLEWVGR
IRSKYNNYATYYADSVKDRFTISRDDSKNSLYLQMNSLKTEDTAVYYCAR
HGNFGNSYVSWFAYWGQGTLVTVSS

The amino acid sequence of hVH9 is shown in SEQ ID NO. 138, of which the encoding nucleic acid is shown in SEQ ID NO. 139, and the HCDR1, HCDR2, and HCDR3 are shown in SEQ ID NOs. 114, 115 and 116, respectively.

EVQLVESGGGLVQPGGSLRLSCAASGFTFNTYAMNWVRKAPGKGLEWVGR
IRSKYNNYATYYADSVKDRFTISRDDSKNSLYLQMNSLKTEDTAVYYCVR
HGNFGNSYVSWFAYWGQGTLVTVSS

The amino acid sequence of hVH10 is shown in SEQ ID NO. 140, of which the encoding nucleic acid is shown in SEQ ID NO. 141, and the HCDR1, HCDR2, and HCDR3 are shown in SEQ ID NOs. 114, 115 and 116, respectively.

EVQLVESGGGLVQPGGSLRLSCAASGFTFNTYAMNWVRKAPGKGLEWVAR
IRSKYNNYATYYADSVKDRFTISRDDSKNSLYLQMNSLKTEDTAVYYCVR
HGNFGNSYVSWFAYWGQGTLVTVSS

After the light and heavy chain humanized variants have been respectively subjected to full sequence synthesis, are cloned into the eukaryotic expression vector containing the constant region of the light chain of antibody λ or the constant region CH1-CH3 of the heavy chain of human IgG4, co-transfected into HEK293E cells, and cultured at 37° C. under 120 rpm 5% CO₂ for 5 to 6 days, and then the culture supernatant is collected and purified by Protein A chromatography column.

### 2. ELISA Affinity Assay

Human CD3εγ protein was coated at 4° C. overnight. After blocking with 2% skim milk, different dilutions of CD3 antibody were added to each well and incubated for 1 h; after the secondary antibody was added to the HPR-labeled goat anti-human IgG Fc and color development of TMB solution, the reaction was stopped with concentrated sulfuric acid and the absorbance was read at 450 nm (the results were shown in FIG. 4).

FIG. 4 shows a combination of anti-CD3 humanized antibodies (including aCD3-hVH8/VL1, aCD3-hVH9/VL1, aCD3-hVH9/VL2, aCD3-hVH9/VL3, aCD3-hVH1/VL5, aCD3-hVH8/VL5, aCD3-hVH9/VL5) binding to human CD3εγ protein, the anti-CD3 humanized antibodies bind to CD3εγ recombinant protein with high affinity.

### 3. Jurkat T Cell Affinity Assay

Jurkat cells in the logarithmic growth phase were blocked with 3% BSA for 30 min, added to 96-well U-plate at 5×10⁴ cells per well, centrifuged to discard the supernatant, and added with 50 μL of gradient diluted antibody (antibody concentration: from 30 μg/mL, 3-fold dilution for 5 gradients) per well, and incubated at 4° C. for 1 h. After washing off the primary antibody, a 1:300 dilution of the secondary antibody, Alexa Fluro647-labeled goat anti-human IgG Fc (Jackson ImmunoResearch, 109-606-170) was added and incubated for 45 min at 4° C. After washing, each well was resuspended in 50 μL PBS for FACS (iQue, Intellicyt) assay (results were shown in FIG. 5).

FIG. 5 shows a combination of anti-CD3 humanized antibodies bound to Jurkat cells, wherein the CD3 humanized antibody hVH9/VL5 (aCD3-hVH9/VL5) binds to Jurkat cells with moderate affinity.

### 4. Screening a Combination of BCMA Antibody and CD3 Antibody

BCMA humanized antibodies h38E2, h23F8, h40C6, h38D9, h29A11, h15H10, and h34G2 (Table 5) were obtained by selecting some BCMA clones with higher affinity and performing CDR transplantation with the human germline genes with the highest homology. Referring to Schaefer W et al. (PNAS, 2011), the combination of humanized BCMA antibody and CD3 antibody aCD3-hVH9/VL5 (the amino acid sequence of the light chain variable region is shown in SEQ ID NO. 106, and the amino acid sequence of the heavy chain variable region is shown in SEQ ID NO. 138) was screened: the light chain variable region of the humanized BCMA antibody was cloned into a eukaryotic expression vector containing the κ light chain constant region, and the heavy chain variable region was cloned into a eukaryotic expression vector containing the mutated human IgG4 constant region; the light chain variable region of humanized CD3 antibody was cloned into a eukaryotic expression vector containing λ light chain constant region as well as heavy chain hinge region and constant regions CH2 and CH3, and the heavy chain variable region of CD3 antibody was cloned into a eukaryotic expression vector containing human CH1 constant region. The four plasmids encoding the light and heavy chain genes were mixed in the ratio of 1:1:1:1 and transfected into HEK293E cells. After expression for 5-6 days, the culture supernatant was collected. After one-step purification by Protein A, SEC-HPLC showed monomer content >55% for relative activity evaluation. BCMA-50 (synthesized according to US201213678247) was constructed as a BCMA50-CD3 bispecific antibody with the CD3 humanized antibody as the method described above, or expressing BCMA50×CD3 (BiTE) with reference to the literature.

TABLE 5
Humanized BCMA antibody variable region sequences

| | LCVR | | LCDR1 | LCDR2 | LCDR3 | HCVR | | HCDR1 | HCDR2 | HCDR3 |
|---|---|---|---|---|---|---|---|---|---|---|
| BCMA | Amino | | Amino | Amino | Amino | Amino | | Amino | Amino | Amino |
| humanized | acid | Nucleotide | acid | acid | acid | acid | Nucleotide | acid | acid | acid |
| antibody | sequence | sequence | Sequence | Sequence | Sequence | Sequence | Sequence | Sequence | Sequence | Sequence |
| h38E2 | 142 | 143 | 144 | 77 | 78 | 145 | 146 | 71 | 147 | 73 |
| h23F8 | 148 | 149 | 17 | 40 | 19 | 150 | 151 | 12 | 50 | 14 |
| h40C6 | 152 | 153 | 92 | 23 | 19 | 154 | 155 | 12 | 13 | 14 |
| h38D9 | 156 | 157 | 83 | 84 | 19 | 158 | 159 | 12 | 13 | 14 |
| h29A11 | 160 | 161 | 17 | 40 | 19 | 162 | 163 | 12 | 13 | 14 |
| h15H10 | 164 | 165 | 17 | 40 | 19 | 166 | 167 | 12 | 36 | 37 |
| h34G2 | 168 | 169 | 17 | 40 | 19 | 170 | 171 | 65 | 66 | 14 |

h38E2

The amino acid sequence of the h38E2 light chain VK is shown in SEQ ID NO. 142, of which the encoding nucleic acid is shown in SEQ ID NO. 143, and the LCDR1, LCDR2, and LCDR3 are shown in SEQ ID NOs. 144, 77 and 78, respectively.

DIQMTQSPSSLSASVGDRVTITCQASQDVDTAVAWYQKKPGKAPKLLIY
WTSTRHTGVPSRFSGSGSGTDFTFTISSLQPEDIATYFCQQFSSYPLTF
GQGTKVEIK
Nucleic acid sequence
GACATCCAGATGACACAGAGCCCTAGCAGCCTGTCTGCCAGCGTGGGAG
ACAGAGTGACCATCACCTGTCAGGCCAGCCAGGATGTGGATACAGCCGT
GGCCTGGTATCAGAAGAAGCCTGGCAAGGCCCCTAAGCTGCTGATCTAC
TGGACCAGCACCAGACACACAGGCGTGCCCTCTAGATTCAGCGGCTCTG
GCTCTGGCACCGACTTCACCTTCACAATCAGCAGCCTGCAGCCTGAGGA
TATCGCTACCTACTTCTGCCAGCAGTTCAGCAGCTACCCTCTGACATTC
GGCCAGGGCACCAAGGTGGAAATCAAG

The amino acid sequence of the h38E2 heavy chain VH is shown in SEQ ID NO. 145, of which the encoding nucleic acid is shown in SEQ ID NO. 146, and the HCDR1, HCDR2, and HCDR3 are shown in SEQ ID NOs. 71, 147 and 73, respectively.

QVQLVQSGAEVκKPGSSVκVSCKASGYTFTNYWIGWVREAPGQGLEWMGD
IYPGSGDTNYNGKFQGRVTLTADISSNTAYMELSSLRSEDTAVYYCARSY
YRNDAASFAYWGQGTLVTVSS
Nucleic acid sequence
CAGGTTCAGCTGGTTCAGTCTGGCGCCGAAGTGAAGAAACCTGGCAGCAG
CGTGAAGGTGTCCTGCAAGGCTAGCGGCTACACATTCACCAACTACTGGA
TCGGCTGGGTCCGAGAGGCTCCTGGACAAGGACTTGAGTGGATGGGCGAC
ATCTACCCTGGCAGCGGCGACACAAACTACAACGGCAAGTTCCAGGGCAG
AGTGACCCTGACAGCCGACATCAGCAGCAACACCGCCTACATGGAACTGA
GCAGCCTGAGAAGCGAGGACACCGCCGTGTACTACTGCGCCAGAAGCTAC
TACAGAAACGACGCCGCCAGCTTCGCTTACTGGGGACAGGGAACACTGGT
CACCGTGTCTAGT
h23F8

The amino acid sequence of the h23F8 light chain VK is shown in SEQ ID NO. 148, of which the encoding nucleic acid is shown in SEQ ID NO. 149, and the LCDR1, LCDR2, and LCDR3 are shown in SEQ ID NOs. 17, 40 and 19, respectively.

DIVLTQSPASLAVSPGQRATITCRASKSVSTSGYSYMHWYQKKPGQPPKL
LIYLASNLESGVPARFSGSGSGTDFTLTINPVEANDTANYYCQHSRELPW
TFGQGTKVEIK
Nucleic acid sequence
GACATCGTGCTGACACAGAGCCCTGCTTCTCTGGCTGTGTCTCCTGGCCA
GAGAGCCACCATCACCTGTAGAGCCAGCAAGAGCGTGTCCACCAGCGGCT
ACTCTTACATGCACTGGTATCAGAAGAAGCCCGGCCAGCCTCCTAAGCTG
CTGATCTACCTGGCTAGCAACCTCGAAAGCGGAGTGCCTGCTAGATTTTC
TGGCAGCGGCTCTGGCACCGACTTCACCCTGACAATCAACCCCGTGGAAG
CCAACGACACCGCCAACTACTACTGCCAGCACAGCAGAGAGCTGCCCTGG
ACATTTGGCCAGGGCACCAAGGTGGAAATCAAG

The amino acid sequence of the h23F8 heavy chain VH is shown in SEQ ID NO. 150, of which the encoding nucleic acid is shown in SEQ ID NO. 151, and the HCDR1, HCDR2, and HCDR3 are shown in SEQ ID NOs. 12, 50 and 14, respectively.

QVQLVQSGSELKKPGASVκVSCKASGYTFTNFGMNWVREAPGQGLEWMGW
INTYTGDQIYADDFKGRFAFSLDTSASTAYLQISSLKAEDTAVYFCARGE
IYYGYDVGFVYWGQGTLVTVSS
Nucleic acid sequence
CAGGTTCAGCTGGTGCAGTCTGGCAGCGAGCTGAAGAAACCTGGCGCCTC
TGTGAAGGTGTCCTGCAAGGCTAGCGGCTACACATTCACCAACTTCGGCA
TGAACTGGGTCCGAGAGGCTCCTGGACAGGGACTCGAATGGATGGGCTGG
ATCAACACCTACACCGGCGACCAGATCTACGCCGACGACTTCAAGGGCAG
ATTCGCCTTCAGCCTGGACACCTCTGCCAGCACAGCTTACCTGCAGATCA
GCAGCCTGAAGGCCGAGGATACCGCCGTGTACTTCTGTGCCAGAGGCGAG
ATCTACTACGGCTACGATGTGGGCTTCGTCTACTGGGGACAGGGAACACT
GGTCACCGTTAGCTCT
h40C6

The amino acid sequence of the h40C6 light chain VK is shown in SEQ ID NO. 152, of which the encoding nucleic acid is shown in SEQ ID NO. 153, and the LCDR1, LCDR2, and LCDR3 are shown in SEQ ID NOs. 92, 23 and 19, respectively.

DIVLTQSPASLAVSPGQRATITCRASRSVTTSGYSYIHWYQKKPGQPPKL
LIYLASDLEAGVPARFSGSGSGTDFTLTINPVEANDTANYYCQHSRELPW
TFGQGTKVEIK
Nucleic acid sequence
GACATCGTGCTGACACAGAGCCCTGCTTCTCTGGCTGTGTCTCCTGGCCA
GAGAGCCACCATCACCTGTAGAGCCAGCAGAAGCGTGACCACCAGCGGCT
ACTCTTACATCCACTGGTATCAGAAGAAGCCCGGCCAGCCTCCTAAGCTG
CTGATCTACCTGGCCAGCGATCTGGAAGCTGGCGTGCCAGCTAGATTTTC
TGGCAGCGGCTCTGGCACCGACTTCACCCTGACAATCAACCCCGTGGAAG
CCAACGACACCGCCAACTACTACTGCCAGCACAGCAGAGAGCTGCCCTGG
ACATTTGGCCAGGGCACCAAGGTGGAAATCAAG

The amino acid sequence of the h40C6 heavy chain VH is shown in SEQ ID NO. 154, of which the encoding nucleic acid is shown in SEQ ID NO. 155, and the HCDR1, HCDR2, and HCDR3 are shown in SEQ ID NOs. 12, 13 and 14, respectively.

QVQLVQSGSELKKPGASVκVSCKASGYIFTNFGMNWVREAPGQGLEWMGW
INTYTGEQIYADDFKGRFAFSLDTSASTAYLQISSLKAEDTAVYFCARGE
IYYGYDVGFVYWGQGTLVTVSS
Nucleic acid sequence
CAGGTTCAGCTGGTGCAGTCTGGCAGCGAGCTGAAGAAACCTGGCGCCTC
TGTGAAGGTGTCCTGCAAGGCTAGCGGCTACATCTTCACCAACTTCGGCA
TGAACTGGGTCCGAGAGGCTCCTGGACAGGGACTCGAATGGATGGGCTGG
ATCAACACCTACACCGGCGAGCAGATCTACGCCGATGACTTCAAAGGCAG
ATTCGCGTTCAGCCTGGACACCAGCGCCAGCACAGCTTACCTGCAGATCA
GCTCTCTGAAGGCCGAGGATACCGCCGTGTACTTCTGTGCCAGAGGCGAG
ATCTACTACGGCTACGACGTGGGCTTTGTGTACTGGGGCCAGGGAACACT
GGTCACCGTTAGCTCT
h38D9

The amino acid sequence of the h38D9 light chain VK is shown in SEQ ID NO. 156, of which the encoding nucleic acid is shown in SEQ ID NO. 157, and the LCDR1, LCDR2, and LCDR3 are shown in SEQ ID NOs. 83, 84 and 19, respectively.

DIVLTQSPASLAVSPGQRATITCRASKSVSTSGYSYMHWYQKKPGQPPKL
LIYLGSDLESGVPARFSGSGSGTDFTLTINPVEANDTANYYCOHSRELPW
TFGQGTKVEIK
Nucleic acid sequence
GACATCGTGCTGACACAGAGCCCTGCTTCTCTGGCTGTGTCTCCTGGCCA
GAGAGCCACCATCACCTGTAGAGCCAGCAAGAGCGTGTCCACCAGCGGCT
ACTCTTACATGCACTGGTATCAGAAGAAGCCCGGCCAGCCTCCTAAGCTG
CTGATCTACCTGGGTAGCGACCTCGAAAGCGGAGTGCCTGCTAGATTTTC
TGGCAGCGGCTCTGGCACCGACTTCACCCTGACAATCAACCCCGTGGAAG
CCAACGACACCGCCAACTACTACTGCCAGCACAGCAGAGAGCTGCCCTGG
ACATTTGGCCAGGGCACCAAGGTGGAAATCAAG

The amino acid sequence of the h38D9 heavy chain VH is shown in SEQ ID NO. 158, of which the encoding nucleic acid is shown in SEQ ID NO. 159, and the HCDR1, HCDR2, and HCDR3 are shown in SEQ ID NOs. 12, 13 and 14, respectively.

QVQLVQSGSELKKPGASVκVSCKASGYIFTNFGMNWVREAPGQGLEWMGW
INTYTGEQIYADDFKGRFAFSLDTSASTAYLQISSLKAEDTAVYFCARGE
IYYGYDVGFVYWGQGTLVTVSS
Nucleic acid sequence
CAGGTTCAGCTGGTGCAGTCTGGCAGCGAGCTGAAGAAACCTGGCGCCTC
TGTGAAGGTGTCCTGCAAGGCTAGCGGCTACATCTTCACCAACTTCGGCA
TGAACTGGGTCCGAGAGGCTCCTGGACAGGGACTCGAATGGATGGGCTGG
ATCAACACCTACACCGGCGAGCAGATCTACGCCGATGACTTCAAAGGCAG
ATTCGCGTTCAGCCTGGACACCAGCGCCAGCACAGCTTACCTGCAGATCA
GCTCTCTGAAGGCCGAGGATACCGCCGTGTACTTCTGTGCCAGAGGCGAG
ATCTACTACGGCTACGACGTGGGCTTTGTGTACTGGGGCCAGGGAACACT
GGTCACCGTTAGCTCT
h29A11

The amino acid sequence of the h29A11 light chain VK is shown in SEQ ID NO. 160, of which the encoding nucleic acid is shown in SEQ ID NO. 161, and the LCDR1, LCDR2, and LCDR3 are shown in SEQ ID NOs. 17, 40 and 19, respectively.

DIVLTQSPASLAVSPGQRATITCRASKSVSTSGYSYMHWYQKKPGQPPKL
LIYLASNLESGVPARFSGSGSGTDFTLTINPVEANDTANYYCQHSRELPW
TFGQGTKVEIK
Nucleic acid sequence
GACATCGTGCTGACACAGAGCCCTGCTTCTCTGGCTGTGTCTCCTGGCCA
GAGAGCCACCATCACCTGTAGAGCCAGCAAGAGCGTGTCCACCAGCGGCT
ACTCTTACATGCACTGGTATCAGAAGAAGCCCGGCCAGCCTCCTAAGCTG
CTGATCTACCTGGCTAGCAACCTCGAAAGCGGAGTGCCTGCTAGATTTTC
TGGCAGCGGCTCTGGCACCGACTTCACCCTGACAATCAACCCCGTGGAAG
CCAACGACACCGCCAACTACTACTGCCAGCACAGCAGAGAGCTGCCCTGG
ACATTTGGCCAGGGCACCAAGGTGGAAATCAAG

The amino acid sequence of h29A11 heavy chain VH is shown in SEQ ID NO. 162, of which the encoding nucleic acid is shown in SEQ ID NO. 163, and the HCDR1, HCDR2, and HCDR3 are shown in SEQ ID NOs. 12, 13 and 14, respectively.

QVQLVQSGSELKKPGASVκVSCKASGYIFTNFGMNWVREAPGQGLEWMGW
INTYTGEQIYADDFKGRFAFSLDTSASTAYLQISSLKAEDTAVYFCARGE
IYYGYDVGFVYWGQGTLVTVSS
Nucleic acid sequence
CAGGTTCAGCTGGTGCAGTCTGGCAGCGAGCTGAAGAAACCTGGCGCCTC
TGTGAAGGTGTCCTGCAAGGCTAGCGGCTACATCTTCACCAACTTCGGCA
TGAACTGGGTCCGAGAGGCTCCTGGACAGGGACTCGAATGGATGGGCTGG
ATCAACACCTACACCGGCGAGCAGATCTACGCCGATGACTTCAAAGGCAG
ATTCGCGTTCAGCCTGGACACCAGCGCCAGCACAGCTTACCTGCAGATCA
GCTCTCTGAAGGCCGAGGATACCGCCGTGTACTTCTGTGCCAGAGGCGAG
ATCTACTACGGCTACGACGTGGGCTTTGTGTACTGGGGCCAGGGAACACT
GGTCACCGTTAGCTCT
h15H10

The amino acid sequence of the h15H10 light chain VK is shown in SEQ ID NO. 164, of which the encoding nucleic acid is shown in SEQ ID NO. 165, and the LCDR1, LCDR2, and LCDR3 are shown in SEQ ID NOs. 17, 40 and 19, respectively.

DIVLTQSPASLAVSPGQRATITCRASKSVSTSGYSYMHWYQKKPGQPPKL
LIYLASNLESGVPARFSGSGSGTDFTLTINPVEANDTANYYCOHSRELPW
TFGQGTKVEIK
Nucleic acid sequence
GACATCGTGCTGACACAGAGCCCTGCTTCTCTGGCTGTGTCTCCTGGCCA
GAGAGCCACCATCACCTGTAGAGCCAGCAAGAGCGTGTCCACCAGCGGCT
ACTCTTACATGCACTGGTATCAGAAGAAGCCCGGCCAGCCTCCTAAGCTG
CTGATCTACCTGGCTAGCAACCTCGAAAGCGGAGTGCCTGCTAGATTTTC
TGGCAGCGGCTCTGGCACCGACTTCACCCTGACAATCAACCCCGTGGAAG
CCAACGACACCGCCAACTACTACTGCCAGCACAGCAGAGAGCTGCCCTGG
ACATTTGGCCAGGGCACCAAGGTGGAAATCAAG

The amino acid sequence of the h15H10 heavy chain VH is shown in SEQ ID NO. 166, of which the encoding nucleic acid is shown in SEQ ID NO. 167, and the HCDR1, HCDR2, and HCDR3 are shown in SEQ ID NOs. 12, 36 and 37, respectively.

QVQLVQSGSELKKPGASVκVSCKASGYTFTNFGMNWVREAPGQGLEWMGW
INTYTGDLIYGDDFKGRFAFSLDTSASTAYLQISSLKAEDTAVYFCARGE
IYYGYDVGFAYWGQGTLVTVSS
Nucleic acid sequence
CAGGTTCAGCTGGTGCAGTCTGGCAGCGAGCTGAAGAAACCTGGCGCCTC
TGTGAAGGTGTCCTGCAAGGCTAGCGGCTACACATTCACCAACTTCGGCA
TGAACTGGGTCCGAGAGGCTCCTGGACAGGGACTCGAATGGATGGGCTGG
ATCAACACCTACACCGGCGACCTGATCTACGGCGACGACTTCAAGGGCAG
ATTCGCCTTCAGCCTGGACACCTCTGCCAGCACAGCTTACCTGCAGATCA
GCAGCCTGAAGGCCGAGGATACCGCCGTGTACTTCTGTGCCAGAGGCGAG
ATCTACTACGGCTACGATGTGGGCTTCGCCTACTGGGGACAGGGAACACT
GGTCACCGTTAGCTCT
h34G2

The amino acid sequence of the h34G2 light chain VK is shown in SEQ ID NO. 168, of which the encoding nucleic acid is shown in SEQ ID NO. 169, and the LCDR1, LCDR2, and LCDR3 are shown in SEQ ID NOs. 17, 40 and 19, respectively.

DIVLTQSPASLAVSPGQRATITCRASKSVSTSGYSYMHWYQKKPGQPPKL
LIYLASNLESGVPARFSGSGSGTDFTLTINPVEANDTANYYCQHSRELPW
TFGQGTKVEIK
Nucleic acid sequence
GACATCGTGCTGACACAGAGCCCTGCTTCTCTGGCTGTGTCTCCTGGCCA
GAGAGCCACCATCACCTGTAGAGCCAGCAAGAGCGTGTCCACCAGCGGCT
ACTCTTACATGCACTGGTATCAGAAGAAGCCCGGCCAGCCTCCTAAGCTG
CTGATCTACCTGGCTAGCAACCTCGAAAGCGGAGTGCCTGCTAGATTTTC
TGGCAGCGGCTCTGGCACCGACTTCACCCTGACAATCAACCCCGTGGAAG
CCAACGACACCGCCAACTACTACTGCCAGCACAGCAGAGAGCTGCCCTGG
ACATTTGGCCAGGGCACCAAGGTGGAAATCAAG

The amino acid sequence of the h34G2 heavy chain VH is shown in SEQ ID NO. 170, of which the encoding nucleic acid is shown in SEQ ID NO. 171, and the HCDR1, HCDR2, and HCDR3 are shown in SEQ ID NOs. 65, 66 and 14, respectively.

QVQLVQSGSELKKPGASVκVSCKASGYTFTNYGMNWVREAPGQGLEWMGW
INTYTGERIYADDFKGRFAFSLDTSASTAYLQISSLKAEDTAVYFCARGE
IYYGYDVGFVYWGQGTLVTVSS
Nucleic acid sequence
CAGGTTCAGCTGGTGCAGTCTGGCAGCGAGCTGAAGAAACCTGGCGCCTC
TGTGAAGGTGTCCTGCAAGGCTAGCGGCTACACATTCACCAACTACGGCA
TGAACTGGGTCCGAGAGGCTCCTGGACAGGGACTCGAATGGATGGGCTGG
ATCAACACCTACACCGGCGAAAGGATCTACGCCGACGACTTCAAGGGCAG
ATTCGCCTTCAGCCTGGACACCTCTGCCAGCACAGCTTACCTGCAGATCA
GCAGCCTGAAGGCCGAGGATACCGCCGTGTACTTCTGTGCCAGAGGCGAG
ATCTACTACGGCTACGATGTGGGCTTCGTCTACTGGGGACAGGGAACACT
GGTCACCGTTAGCTCT

### 5. T-Cell-Dependent Cellular Cytotoxity Assay (TDCC)

Freshly isolated PBMC was mixed with RPMI-8226 cells in the logarithmic growth phase (effector/target cell=10:1), and 50 μL of gradient diluted antibody (initial concentration 40 μg/mL, 10-fold dilution for 7 gradients) was added to each well, and cultured at 37° C. in 5% CO₂ for 24 h. After the end of the culture, centrifuging at 1000 rpm for 3 min, the supernatant was taken to detect the release of LDH, transferred 50 μL of the supernatant to a black ELISA plate, added with 50 μL/well of LDH detection substrate, 10 min later, stop the reaction and detect (Biotek Synergy HT), and the killing rate was calculated according to the following formula; after discarding the supernatant, the remaining cells in the wells were washed twice with 4% calf blood, blocked with 100 μg/mL human IgG for 10 min, and T cell early activation marker CD69 and late marker CD25 were added to detect antibodies (CD25-PE, CD4-APC, CD69-FITC, and CD8-APC) and incubating on ice for 20 min. After the incubation, 200 μL of 4% calf blood was added to each well for washing three times, the supernatant was discarded, 60 μL/well propidium iodide (PI) solution (1:200 dilution) was added, incubated on ice for 5 min, and detected by flow cytometer (BD FACS celesta). The formula for calculating the killing rate is as follows:

$$Killing Rate ( \% ) = \frac{Experimental well - Target cell spontaneous}{Target cell maximum release - Target cell spontaneous} \times 100 \%$$

The killing activity of the bispecific antibody combination is shown in Table 6.

TABLE 6
Killing activity of bispecific antibodies

| | SEC- | | Upregulation of CD69 | | Upregulation of CD25 | |
|---|---|---|---|---|---|---|
| | HPLC | | EC₅₀ | | EC₅₀ | |
| Antibodies\Units | Monomer | Killing | (%) | | (%) | |
| (nM) | % | EC₅₀ | CD8⁺T | CD4⁺T | CD8⁺T | CD4⁺T |
| h38E2-CD3 | 56.2 | 13.64 | 2.32(69) | 7.40(55) | 3.65(38) | 3.65(29) |
| h23F8-CD3 | 58.4 | 0.81 | 0.49(59) | 0.44(41) | 0.79(34) | 0.57(25) |
| h40C6-CD3 | 57.3 | 1.12 | 0.21(44) | 0.63(37) | 0.49(28) | 0.45(25) |
| h38D9-CD3 | 55.1 | 1.04 | 0.37(52) | 0.52(57) | 1.75(35) | 0.77(29) |
| h29A11-CD3 | 56.3 | 0.69 | 3.39(58) | 0.37(47) | 3.85(36) | 0.45(33) |
| h15H10-CD3 | 67.6 | 1.83 | 0.94(62) | 3.39(58) | 2.64(41) | 3.85(36) |
| h34G2-CD3 | 64.0 | 1.02 | 0.57(55) | 0.68(42) | 1.28(33) | 1.18(27) |
| BCMA50-CD3 | 40.9 | 3.84 | 6.51(56) | 1.02(34) | 9.86(24) | 7.94(22) |
| BCMA50 × CD3(BiTE) | N.D. | 0.81 | 0.16(65) | 1.90(52) | 0.30(39) | 0.64(27) |
| KLH-CD3 | 85.8 | — | —(11) | —(15) | —(8) | —(10) |

### 6. Construction of BCMA×CD3 κλ Humanized Bispecific Antibody

Clone 29A11 with higher killing efficiency and clone 6G with homologous sequence were selected for humanized sequence optimization. The heavy chain variable region was derived from murine IGHV9-3, and the light chain variable region was derived from murine IGKV3-12. The light and heavy chain CDR grafts were transplanted into human germline genes IGHV7-4-1*02 and IGVK_7_3, respectively. The humanized BCMA antibody sequences were synthesized and designed. Meanwhile, charge variants were introduced in the BCMA antigen arm and the humanized CD3 antibody arm containing the λ light chain (Vκ_{BCMA}: Gln₄₂Lys; VH_{BCMA}: Gln₃₉Glu; Vλ_{CD3}: Gln₄₀Glu; VH_{CD3}: Gln₃₉Lys) to construct a novel BCMA-CD3 κλ humanized bispecific antibody with the native IgG configuration (Table 7). The Fc portion of the bispecific antibody adopts the human IgG4 knob-into-hole structure to achieve heterodimer pairing (Atwell et al. 1997), and through mutations Ser₂₂₈Pro, Leu₂₃₅Glu, and Pro₃₂₉Ala, the hinge region is kept stable and interactions with Fcγ receptors and C1q are reduced.

Plasmids encoding the corresponding antibody fragments were mixed in a ratio of κ light chain:λ light chain:heavy chain 1:heavy chain 2=2:2:1:1 with 3 mg/mL PEI, then co-transfected into CHO-S cells and cultured in 500 mL CD CHO AGT medium (Gibco #12490-001) at 37° C., 5% CO₂ 150 rpm. 4% CHO Feed C+ supplement (Gibco #A25031-05) was added on the 2^{nd}, 4^{th} and 6^{th} day of transient transfection, respectively. When the cell viability decreased to about 85%, the fermentation broth was harvested, filtered, and purified by Protein A affinity chromatography, and the SEC-HPLC monomer content was higher than 92%; through Capto S ImpAct ion exchange chromatography, 50-300 mM NaCl/phosphate pH6.0 gradient elution and combined elution peak, the monomer content was further increased to more than 98-99% (Table 8).

TABLE 7
BCMA × CD3 κλ bispecific antibodies

| Bispecific | | |
|---|---|---|
| antibody | BCMA antibody | CD3 antibody |
| BCMA × CD3 | Light chain 1: the amino acid | Light chain 2: the amino acid |
| κλ003, | sequence thereof is shown in SEQ | sequence thereof is shown in SEQ |
| introduction of | ID NO. 172, and the encoding | ID NO. 180, and the encoding |
| charge variants | nucleic acid thereof is shown in | nucleic acid thereof is shown in |
| in the BCMA and | SEQ ID NO. 173 | SEQ ID NO. 181 |
| CD3 antigen arms: | DIVLTQSPASLAVSPGQRATIT | QAVVTQEPSLTVSPGGTVTLTC |
| Vκ_{BCMA}: Gln₄₂Lys; | CRASKSVSTSGYSYMHWYQK | RSSTGAVTTSNYANWVQEKPG |
| VH_{BCMA}: Gln₃₉Glu; | KPGQPPKLLIYLASNLESGVP | QAPRGLIGGTNKRAPWTPARF |
| Vλ_{CD3}: Gln₄₀Glu; | ARFSGSGSGTDFTLTINPVEAE | SGSLLGGKAALTITGAQAEDE |
| VH_{CD3}: Gln₃₉Lys | DTANYYCQHSRELPWTFGQG | AEYYCVLWYSNLWVFGGGTK |
| | TKVEIKRTVAAPSVFIFPPSDE | LTVLGQPKAAPSVTLFPPSSEE |
| | QLKSGTASVVCLLNNFYPREA | LQANKATLVCLISDFYPGAVTV |
| | KVQWKVDNALQSGNSQESV | AWKADSSPVκAGVETTTPSKQ |
| | TEQDSKDSTYSLSSTLTLSKA | SNNKYAASSYLSLTPEQWKSH |
| | DYEKHKVYACEVTHQGLSSP | RSYSCQVTHEGSTVEKTVAPT |
| | VTKSFNRGEC | ECS |
| | Heavy chain 1: the amino acid | Heavy chain 2: the amino acid |
| | sequence thereof is shown in SEQ | sequence thereof is shown in SEQ |
| | ID NO. 174, and the encoding | ID NO. 182, and the encoding |
| | nucleic acid thereof is shown in | nucleic acid thereof is shown in |
| | SEQ ID NO. 175 | SEQ ID NO. 183 |
| | QVQLVQSGSELKKPGASVKV | EVOLVESGGGLVQPGGSLRLS |
| | SCKASGYIFTNFGMNWVREA | CAASGFTFNTYAMNWVRKAP |
| | PGQGLEWMGWINTYTGEQIY | GKGLEWVGRIRSKYNNYATYY |
| | ADGFTGRFVFSLDTSASTAYL | ADSVKDRFTISRDDSKNSLYLQ |
| | QISSLKAEDTAVYFCARGEIY | MNSLKTEDTAVYYCVRHGNF |
| | YGYDVGFVYWGQGTLVTVSS | GNSYVSWFAYWGQGTLVTVS |
| | ASTKGPSVFPLAPCSRSTSEST | SASTKGPSVFPLAPCSRSTSEST |
| | AALGCLVKDYFPEPVTVSWN | AALGCLVKDYFPEPVTVSWNS |
| | SGALTSGVHTFPAVLQSSGLYS | GALTSGVHTFPAVLQSSGLYSL |
| | LSSVVTVPSSSLGTKTYTCNV | SSVVTVPSSSLGTKTYTCNVD |
| | DHKPSNTKVDKRVESKYGPP | HKPSNTKVDKRVESKYGPPCP |
| | CPPCPAPEFEGGPSVFLFPPKP | PCPAPEFEGGPSVFLFPPKPKD |
| | KDTLMISRTPEVTCVVVDVSQ | TLMISRTPEVTCVVVDVSQEDP |
| | EDPEVQFNWYVDGVEVHNA | EVQFNWYVDGVEVHNAKTKP |
| | KTKPREEQFNSTYRVVSVLTV | REEQFNSTYRVVSVLTVLHQD |
| | LHQDWLNGKEYKCKVSNKG | WLNGKEYKCKVSNKGLASSIE |
| | LASSIEKTISKAKGQPREPQVC | KTISKAKGQPREPQVYTLPPCQ |
| | TLPPSQEEMTKNQVSLSCAVκ | EEMTKNQVSLWCLVκGFYPSD |
| | GFYPSDIAVEWESNGQPENNY | IAVEWESNGQPENNYKTTPPV |
| | KTTPPVLDSDGSFFLVSKLTV | LDSDGSFFLYSKLTVDKSRWQ |
| | DKSRWQEGNVFSCSVMHEAL | EGNVFSCSVMHEALHNHYTQ |
| | HNHYTQKSLSLSLGK | KSLSLSLGK |
| BCMA × CD3 | Light chain 1: the amino acid | Light chain 2: the amino acid |
| κλ004 | sequence thereof is shown in SEQ | sequence thereof is shown in SEQ |
| introduction of | ID NO. 176, and the encoding | ID NO. 180, and the encoding |
| charge variants | nucleic acid thereof is shown in | nucleic acid thereof is shown in |
| in the BCMA and | SEQ ID NO. 177 | SEQ ID NO. 181 |
| CD3 antigen arms: | DIVLTQSPASLAVSPGQRATIT | QAVVTQEPSLTVSPGGTVTLTC |
| Vκ_{BCMA}: Gln₄₂Lys; | CRASKSVTTSGYSYIHWYQK | RSSTGAVTTSNYANWVQEKPG |
| VH_{BCMA}: Gln₃₉Glu; | KPGQPPKLLIYLASDLEAGVP | QAPRGLIGGTNKRAPWTPARF |
| Vλ_{CD3}: Gln₄₀Glu; | ARFSGSGSGTDFTLTINPVEAE | SGSLLGGKAALTITGAQAEDE |
| VH_{CD3}: Gln₃₉Lys | DTANYYCQHSRELPWTFGQG | AEYYCVLWYSNLWVFGGGTK |
| | TKVEIKRTVAAPSVFIFPPSDE | LTVLGQPKAAPSVTLFPPSSEE |
| | QLKSGTASVVCLLNNFYPREA | LQANKATLVCLISDFYPGAVTV |
| | KVQWKVDNALQSGNSQESV | AWKADSSPVKAGVETTTPSKQ |
| | TEQDSKDSTYSLSSTLTLSKA | SNNKYAASSYLSLTPEQWKSH |
| | DYEKHKVYACEVTHQGLSSP | RSYSCQVTHEGSTVEKTVAPT |
| | VTKSFNRGEC | ECS |
| | Heavy chain 1: the amino acid | Heavy chain 2: the amino acid |
| | sequence thereof is shown in SEQ | sequence thereof is shown in SEQ |
| | ID NO. 174, and the encoding | ID NO. 182, and the encoding |
| | nucleic acid thereof is shown in | nucleic acid thereof is shown in |
| | SEQ ID NO. 175 | SEQ ID NO. 183 |
| | QVQLVQSGSELKKPGASVKV | EVOLVESGGGLVQPGGSLRLS |
| | SCKASGYIFTNFGMNWVREA | CAASGFTFNTYAMNWVRKAP |
| | PGQGLEWMGWINTYTGEQIY | GKGLEWVGRIRSKYNNYATYY |
| | ADGFTGRFVFSLDTSASTAYL | ADSVKDRFTISRDDSKNSLYLQ |
| | QISSLKAEDTAVYFCARGEIY | MNSLKTEDTAVYYCVRHGNF |
| | YGYDVGFVYWGQGTLVTVSS | GNSYVSWFAYWGQGTLVTVS |
| | ASTKGPSVFPLAPCSRSTSEST | SASTKGPSVFPLAPCSRSTSEST |
| | AALGCLVKDYFPEPVTVSWN | AALGCLVKDYFPEPVTVSWNS |
| | SGALTSGVHTFPAVLQSSGLYS | GALTSGVHTFPAVLQSSGLYSL |
| | LSSVVTVPSSSLGTKTYTCNV | SSVVTVPSSSLGTKTYTCNVD |
| | DHKPSNTKVDKRVESKYGPP | HKPSNTKVDKRVESKYGPPCP |
| | CPPCPAPEFEGGPSVFLFPPKP | PCPAPEFEGGPSVFLFPPKPKD |
| | KDTLMISRTPEVTCVVVDVSQ | TLMISRTPEVTCVVVDVSQEDP |
| | EDPEVQFNWYVDGVEVHNA | EVQFNWYVDGVEVHNAKTKP |
| | KTKPREEQFNSTYRVVSVLTV | REEQFNSTYRVVSVLTVLHQD |
| | LHQDWLNGKEYKCKVSNKG | WLNGKEYKCKVSNKGLASSIE |
| | LASSIEKTISKAKGQPREPQVC | KTISKAKGQPREPQVYTLPPCQ |
| | TLPPSQEEMTKNQVSLSCAVκ | EEMTKNQVSLWCLVκGFYPSD |
| | GFYPSDIAVEWESNGQPENNY | IAVEWESNGQPENNYKTTPPV |
| | KTTPPVLDSDGSFFLVSKLTV | LDSDGSFFLYSKLTVDKSRWQ |
| | DKSRWQEGNVFSCSV | EGNVFSCSVMHEALHNHYTQ |
| | MHEALHNHYTQKSLSLSLGK | KSLSLSLGK |
| BCMA × CD3 | Light chain 1: the amino acid | Light chain 2: the amino acid |
| κλ005 | sequence thereof is shown in SEQ | sequence thereof is shown in SEQ |
| introduction of | ID NO. 172, and the encoding | ID NO. 180, and the encoding |
| charge variants in | nucleic acid thereof is shown in | nucleic acid thereof is shown in |
| the BCMA and | SEQ ID NO. 173 | SEQ ID NO. 181 |
| CD3 antigen arms: | DIVLTQSPASLAVSPGQRATIT | QAVVTQEPSLTVSPGGTVTLTC |
| Vκ_{BCMA}: Gln₄₂Lys; | CRASKSVSTSGYSYMHWYQK | RSSTGAVTTSNYANWVQEKPG |
| VH_{BCMA}: Gln₃₉Glu; | KPGQPPKLLIYLASNLESGVP | QAPRGLIGGTNKRAPWTPARF |
| Vλ_{CD3}: Gln₄₀Glu; | ARFSGSGSGTDFTLTINPVEAE | SGSLLGGKAALTITGAQAEDE |
| VH_{CD3}: Gln₃₉Lys | DTANYYCQHSRELPWTFGQG | AEYYCVLWYSNLWVFGGGTK |
| | TKVEIKRTVAAPSVFIFPPSDE | LTVLGQPKAAPSVTLFPPSSEE |
| | QLKSGTASVVCLLNNFYPREA | LQANKATLVCLISDFYPGAVTV |
| | KVQWKVDNALQSGNSQESV | AWKADSSPVKAGVETTTPSKQ |
| | TEQDSKDSTYSLSSTLTLSKA | SNNKYAASSYLSLTPEQWKSH |
| | DYEKHKVYACEVTHQGLSSP | RSYSCQVTHEGSTVEKTVAPT |
| | VTKSFNRGEC | ECS |
| | Heavy chain 1: the amino acid | Heavy chain 2: the amino acid |
| | sequence thereof is shown in SEQ | sequence thereof is shown in SEQ |
| | ID NO. 178, and the encoding | ID NO. 182, and the encoding |
| | nucleic acid thereof is shown in | nucleic acid thereof is shown in |
| | SEQ ID NO. 179 | SEQ ID NO. 183 |
| | QVQLVQSGSELKKPGASVκV | EVQLVESGGGLVQPGGSLRLS |
| | SCKASGYIFTNFGMNWVREA | CAASGFTFNTYAMNWVRKAP |
| | PGQGLEWMGWINTYTGEQIY | GKGLEWVGRIRSKYNNYATYY |
| | ADGFTGRFVFSLDTSVSTAYL | ADSVKDRFTISRDDSKNSLYLQ |
| | QISSLKAEDTAVYFCARGEIY | MNSLKTEDTAVYYCVRHGNF |
| | YGYDVGFVYWGQGTLVTVSS | GNSYVSWFAYWGQGTLVTVS |
| | ASTKGPSVFPLAPCSRSTSEST | SASTKGPSVFPLAPCSRSTSEST |
| | AALGCLVKDYFPEPVTVSWN | AALGCLVKDYFPEPVTVSWNS |
| | SGALTSGVHTFPAVLQSSGLYS | GALTSGVHTFPAVLQSSGLYSL |
| | LSSVVTVPSSSLGTKTYTCNV | SSVVTVPSSSLGTKTYTCNVD |
| | DHKPSNTKVDKRVESKYGPP | HKPSNTKVDKRVESKYGPPCP |
| | CPPCPAPEFEGGPSVFLFPPKP | PCPAPEFEGGPSVFLFPPKPKD |
| | KDTLMISRTPEVTCVVVDVSQ | TLMISRTPEVTCVVVDVSQEDP |
| | EDPEVQFNWYVDGVEVHNA | EVQFNWYVDGVEVHNAKTKP |
| | KTKPREEQFNSTYRVVSVLTV | REEQFNSTYRVVSVLTVLHQD |
| | LHQDWLNGKEYKCKVSNKG | WLNGKEYKCKVSNKGLASSIE |
| | LASSIEKTISKAKGQPREPQVC | KTISKAKGQPREPQVYTLPPCQ |
| | TLPPSQEEMTKNQVSLSCAVκ | EEMTKNQVSLWCLVκGFYPSD |
| | GFYPSDIAVEWESNGQPENNY | IAVEWESNGQPENNYKTTPPV |
| | KTTPPVLDSDGSFFLVSKLTV | LDSDGSFFLYSKLTVDKSRWQ |
| | DKSRWQEGNVFSCSVMHEAL | EGNVFSCSVMHEALHNHYTQ |
| | HNHYTQKSLSLSLGK | KSLSLSLGK |
| BCMA × CD3 | Light chain 1: the amino acid | Light chain 2: the amino acid |
| κλ006 | sequence thereof is shown in SEQ | sequence thereof is shown in SEQ |
| introduction of | ID NO. 176, and the encoding | ID NO. 180, and the encoding |
| charge variants | nucleic acid thereof is shown in | nucleic acid thereof is shown in |
| in the BCMA and | SEQ ID NO. 177 | SEQ ID NO. 181 |
| CD3 antigen arms: | DIVLTQSPASLAVSPGQRATIT | QAVVTQEPSLTVSPGGTVTLTC |
| Vκ_{BCMA}: Gln₄₂Lys; | CRASKSVTTSGYSYIHWYQK | RSSTGAVTTSNYANWVQEKPG |
| VH_{BCMA}: Gln₃₉Glu; | KPGQPPKLLIYLASDLEAGVP | QAPRGLIGGTNKRAPWTPARF |
| Vλ_{CD3}: Gln₄₀Glu; | ARFSGSGSGTDFTLTINPVEAE | SGSLLGGKAALTITGAQAEDE |
| VH_{CD3}: Gln₃₉Lys | DTANYYCQHSRELPWTFGQG | AEYYCVLWYSNLWVFGGGTK |
| | TKVEIKRTVAAPSVFIFPPSDE | LTVLGQPKAAPSVTLFPPSSEE |
| | QLKSGTASVVCLLNNFYPREA | LQANKATLVCLISDFYPGAVTV |
| | KVQWKVDNALQSGNSQESV | AWKADSSPVKAGVETTTPSKQ |
| | TEQDSKDSTYSLSSTLTLSKA | SNNKYAASSYLSLTPEQWKSH |
| | DYEKHKVYACEVTHQGLSSP | RSYSCQVTHEGSTVEKTVAPT |
| | VTKSFNRGEC | ECS |
| | Heavy chain 1: the amino acid | Heavy chain 2: the amino acid |
| | sequence thereof is shown in SEQ | sequence thereof is shown in SEQ |
| | ID NO. 178, and the encoding | ID NO. 182, and the encoding |
| | nucleic acid thereof is shown in | nucleic acid thereof is shown in |
| | SEQ ID NO. 179 | SEQ ID NO. 183 |
| | QVQLVQSGSELKKPGASVKV | EVQLVESGGGLVQPGGSLRLS |
| | SCKASGYIFTNFGMNWVREA | CAASGFTFNTYAMNWVRKAP |
| | PGQGLEWMGWINTYTGEQIY | GKGLEWVGRIRSKYNNYATYY |
| | ADGFTGRFVFSLDTSVSTAYL | ADSVκDRFTISRDDSKNSLYLQ |
| | QISSLKAEDTAVYFCARGEIY | MNSLKTEDTAVYYCVRHGNF |
| | YGYDVGFVYWGQGTLVTVSS | GNSYVSWFAYWGQGTLVTVS |
| | ASTKGPSVFPLAPCSRSTSEST | SASTKGPSVFPLAPCSRSTSEST |
| | AALGCLVKDYFPEPVTVSWN | AALGCLVKDYFPEPVTVSWNS |
| | SGALTSGVHTFPAVLQSSGLYS | GALTSGVHTFPAVLQSSGLYSL |
| | LSSVVTVPSSSLGTKTYTCNV | SSVVTVPSSSLGTKTYTCNVD |
| | DHKPSNTKVDKRVESKYGPP | HKPSNTKVDKRVESKYGPPCP |
| | CPPCPAPEFEGGPSVFLFPPKP | PCPAPEFEGGPSVFLFPPKPKD |
| | KDTLMISRTPEVTCVVVDVSQ | TLMISRTPEVTCVVVDVSQEDP |
| | EDPEVQFNWYVDGVEVHNA | EVQFNWYVDGVEVHNAKTKP |
| | KTKPREEQFNSTYRVVSVLTV | REEQFNSTYRVVSVLTVLHQD |
| | LHQDWLNGKEYKCKVSNKG | WLNGKEYKCKVSNKGLASSIE |
| | LASSIEKTISKAKGQPREPQVC | KTISKAKGQPREPQVYTLPPCQ |
| | TLPPSQEEMTKNQVSLSCAVκ | EEMTKNQVSLWCLVκGFYPSD |
| | GFYPSDIAVEWESNGQPENNY | IAVEWESNGQPENNYKTTPPV |
| | KTTPPVLDSDGSFFLVSKLTV | LDSDGSFFLYSKLTVDKSRWQ |
| | DKSRWQEGNVFSCSVMHEAL | EGNVFSCSVMHEALHNHYTQ |
| | HNHYTQKSLSLSLGK | KSLSLSLGK |

TABLE 8
Purity of BCMA × CD3 κλ bispecific antibody

| | HPLC-SEC | | | HPLC-SEC | | |
|---|---|---|---|---|---|---|
| HPLC | (after Protein A) | | | (after cation exchange chromatography) | | |
| detection | Mer | Monomer | Fragment | Mer | Monomer | Fragment |
| BCMA × CD3 κλ003 | 2.3% | 94.4% | 3.3% | 1.8% | 98.2% | 0.0% |
| BCMA × CD3 κλ004 | 1.4% | 95.2% | 3.4% | 1.0% | 99.0% | 0.0% |
| BCMA × CD3 κλ005 | 2.0% | 91.9% | 6.2% | 1.6% | 98.4% | 0.0% |
| BCMA × CD3 κλ006 | 0.5% | 96.4% | 3.1% | 0.6% | 99.4% | 0.0% |

### Example 4. Binding Activity of BCMA×CD3 κλ Bispecific Antibody

The affinity of the bispecific antibody BCMA antigen arm was determined by measuring binding to BCMA recombinant protein, overexpressing stably transfected cells, or BCMA+ tumor cells, respectively, and the affinity of the bispecific antibody CD3 arm was determined by measuring binding to CD3 recombinant antigen, Jurkat cells, or freshly isolated peripheral blood T cells.

### 1. Affinity Assay of BCMA×CD3 κλ Bispecific Antibody to Antigen

10 μg/mL human or cynomolgus monkey BCMA or CD3εγ recombinant antigen was bound to a CM5 chip (GE healthcare) by amino coupling, controlling the amount of antigen bound to approximately 200 RU. After the baseline plateau, gradient-diluted antibodies (from 10 μg/mL, 2-fold dilutions for 7 gradients) flowed through the chip at a flow rate of 30 μL/min for a binding time of 350 seconds and a dissociation time of 600 seconds. Kinetic constants were fitted using Biacore T200 evaluation software with a 1:1 binding model. The results of the affinity assay are shown in Table 9.

TABLE 9
Affinity of BCMA × CD3 κλ bispecific antibodies
to BCMA and CD3 recombinant antigens

| | | Cynomolgus | | Cynomolgus |
|---|---|---|---|---|
| | Human | monkey | Human | monkey |
| KD (nM) | BCMA | BCMA | CD3εγ | CD3εγ |
| BCMA × CD3 | 1.3 | 0.16 | 31 | 43 |
| κλ003 | | | | |
| BCMA × CD3 | 1.0 | 0.12 | 32 | 43 |
| κλ004 | | | | |
| BCMA × CD3 | 1.1 | 0.09 | 28 | 20 |
| κλ005 | | | | |
| BCMA × CD3 | 1.2 | 0.10 | 27 | 33 |
| κλ006 | | | | |

### 2. Binding of BCMA×CD3 κλ Bispecific Antibody to CMA Stably Transected Cells

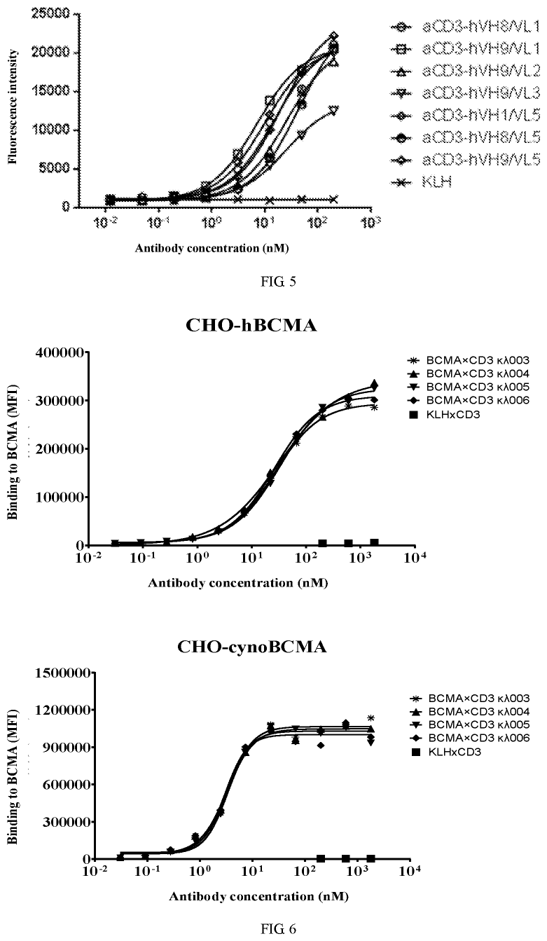

CHO-human BCMA and CHO-cynomolgus monkey BCMA stably transfected cells in the logarithmic growth phase prepared in Example 1 were adjusted to 5×10⁵ cells/ml with 4% calf serum (Hyclone, SH30626.06), 100 μl/well cell suspension was added to a 96-well U-plate, centrifuged at 300 g for 5 min, the supernatant was discarded, and 100 μL of gradient diluted antibody (initial concentration 1800 nM, 3-fold dilution for 10 gradients) was added to each well and incubated at 4° C. for 60 min. 50 μL/well Alexa Fluro647 labeled goat anti-human IgG Fc (1:300 dilution) was added to the secondary antibody, incubated on ice for 20 min, 50 μL/well PI solution (1:300) was added after washing once, incubated for 5 min, and flow cytometer was used to perform detection. FIG. 6 and Table 10 show that the BCMA×CD3 κλ bispecific antibody binds to human, cynomolgus monkey BCMA stably transfected cells with high affinity.

TABLE 10
Binding of BCMA × CD3 κλ bispecific antibodies
to BCMA stably transfected cells

| | | Cynomolgus |
|---|---|---|
| | Human | monkey |
| EC₅₀ (nM) | BCMA-CHO | BCMA-CHO |
| BCMA × CD3 κλ003 | 16 | 3 |
| BCMA × CD3 κλ004 | 22 | 3 |
| BCMA × CD3 κλ005 | 24 | 3 |
| BCMA × CD3 κλ006 | 16 | 3 |
| KLH × CD3 | — | — |

### 3. Binding of BCMA×CD3 xi Bispecific Antibody to BCMA⁺ Tumor Cells

NCI-H929 and RPMI-8226 cells in the logarithmic growth phase were added with 200 μg/mL murine IgG (Jackson ImmunoResearch, 115-005-03), blocked with an ice bath for 30 min, adjusted cells to 5×10⁵ cells/mL with 4% calf serum, 100 μL/well was added to 96-well U-plate, centrifuged at 300 g for 5 min, the supernatant was discarded, and 100 μL of gradient diluted antibody (initial concentration 1800 nM, 3-fold dilution for 10 gradients) was added to each well and incubated at 4° C. for 60 min. The primary antibody was washed off, 50 μL/well Alexa Fluro647 labeled goat anti-human IgG Fc (1:300 dilution) was added, incubated on ice for 20 min, 50 μL/well PI was added after washing once, incubated for 5 min, and flow cytometer was used to perform detection.

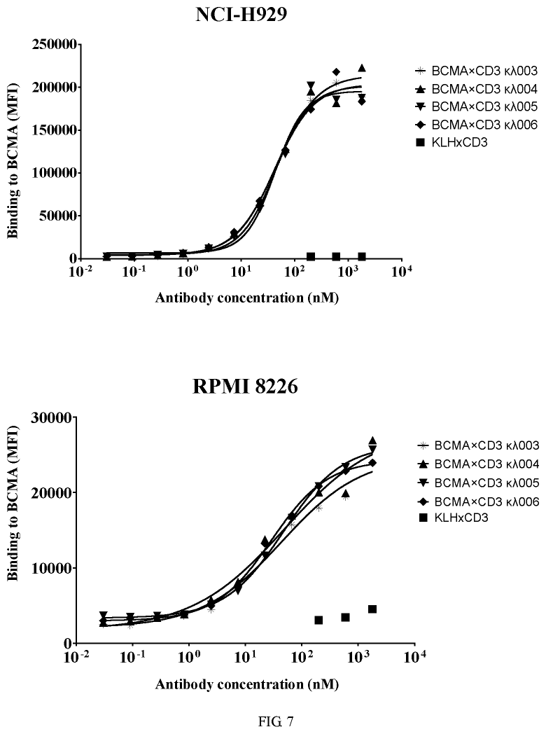

The results are shown in FIG. 7 and Table 11. the BCMA×CD3 κλ bispecific antibody binds to BCMA+ tumor cells NCI-H929 and RPMI-8226 with high affinity.

TABLE 11
Binding of BCMA × CD3 κλ bispecific antibodies
to BCMA+ tumor cells

| EC₅₀(nM) | NCI-H929 | RPMI-8226 |
|---|---|---|
| BCMA × CD3 κλ003 | 34 | 34 |
| BCMA × CD3 κλ004 | 36 | 39 |
| BCMA × CD3 κλ005 | 33 | 38 |
| BCMA × CD3 κλ006 | 32 | 19 |
| KLH × CD3 | — | — |

### 4. Binding of BCMA×CD3 κλ Bispecific Antibody to Jurkat Cells

Jurkat cells in the logarithmic growth phase were added with 200 μg/mL murine IgG (Jackson ImmunoResearch, 115-005-03) and incubated on ice for 30 min. Adjusted cells to 5×10⁵ cells/mL with 4% calf serum, 100 μL/well was added to 96-well U-plate, centrifuged at 300 g to remove the supernatant, and 100 μL of gradient diluted antibody (initial concentration 1800 nM, 3-fold dilution for 10 gradients) was added to each well and incubated at 4° C. for 60 min. 50 μL/well Alexa Fluro647 labeled goat anti-human IgG Fc (1:300 dilution) was added to the secondary antibody, incubated on ice for 20 min, 50 μL/well PI was added after washing once, incubated for 5 min, and flow cytometer (BD C6) was used to perform detection. The results are shown in FIG. 8 and Table 12. The BCMA×CD3 κλ bispecific antibody binds to human leukemia T cell line Jurkat cells with moderate affinity.

TABLE 12
Binding of BCMA × CD3 κλ bispecific antibodies to Jurkat cells

| EC₅₀(nM) | Jurkat |
|---|---|
| BCMA × CD3 κλ003 | 66 |
| BCMA × CD3 κλ004 | 61 |
| BCMA × CD3 κλ005 | 51 |
| BCMA × CD3 κλ006 | 56 |
| KLH × CD3 | 76 |

### 5. Binding of BCMA×CD3 κλ Bispecific Antibody to Peripheral Blood T Cells

Fresh human or cynomolgus monkey peripheral blood was taken and PBMC was isolated by Ficoll-Paque Plu (GE, 17-1440-03). PBMC was adjusted to 5×10⁵ cells/mL with 4% calf serum (Hyclone, SH30626.06), 100 μL/well was added to a 96-well U-plate, centrifuged to remove the supernatant and 100 μL of gradient diluted antibody (initial concentration 1800 nM, 3-fold dilution, 10 gradients) was added to each well and incubated at 4° C. for 60 min. 50 μL/well Alexa Fluro647 labeled goat anti-human IgG Fc (1:300 dilution) was added to the secondary antibody, ice bath for 20 min, 50 μL/well PI was added after washing once, incubated for 5 min, and flow cytometer (BD C6) was used to perform detection. The control antibody REGN5458 was synthesized and prepared according to US20200024356.

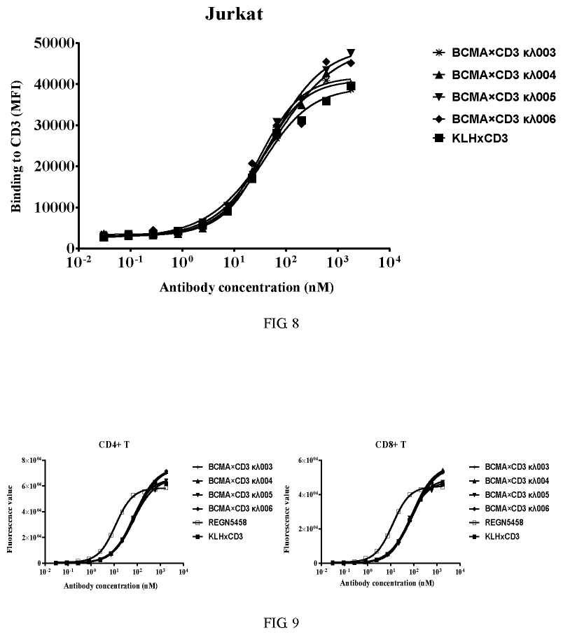

As shown in FIG. 9 and Table 13, the BCMA×CD3 κλ bispecific antibody recognizes human peripheral blood CD4+ T and CD8+ T cells with an affinity of about 60-97 nM, which is weaker than the binding force of the BCMA antigen arm to the BCMA receptor, so that the bispecific antibody preferentially enriches into tumor cells.

TABLE 13
Binding of BCMA × CD3 κλ bispecific
antibodies to peripheral blood T cells

| EC₅₀(nM) | CD4+ T cell | CD8+ T cell |
|---|---|---|
| BCMA × CD3 κλ003 | 87 | 97 |
| BCMA × CD3 κλ004 | 77 | 84 |
| BCMA × CD3 κλ005 | 66 | 60 |
| BCMA × CD3 κλ006 | 91 | 93 |
| KLH × CD3 | 57 | 62 |

### Example 5. BCMA×CD3 κλ Bispecific Antibody-Mediated TDCC

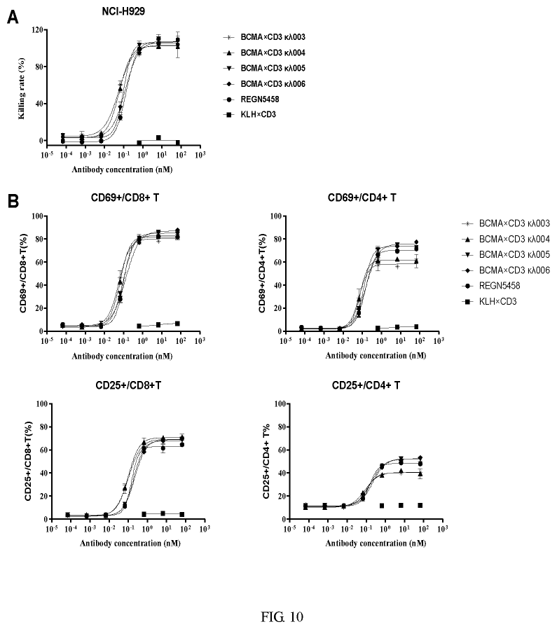

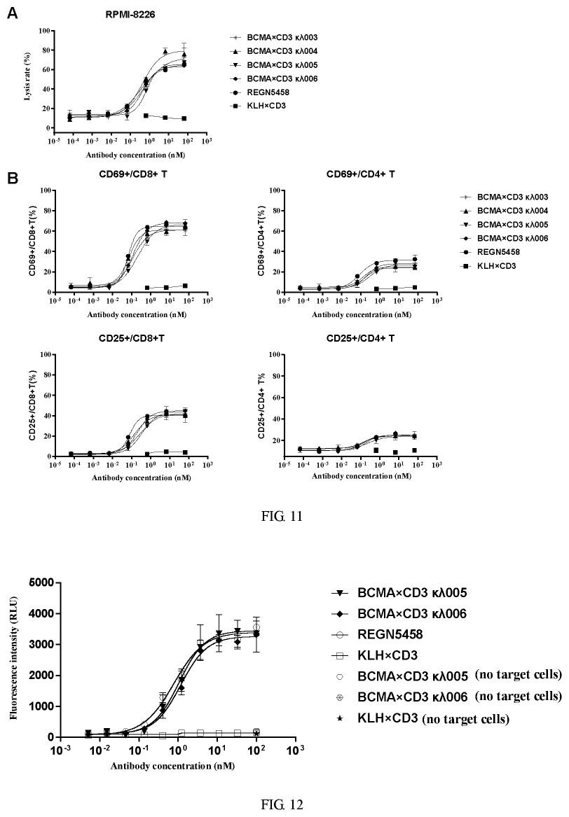

Freshly isolated PBMC was mixed with target cells, NCI-H929 and RPMI-8226 cells, in the logarithmic growth phase, respectively, with effect/target cell=8:1, 50 μL of gradient diluted antibody (antibody concentration from 66.7 nM, 10-fold dilution for 7 gradients) was added to each well and cultured at 37° C. in 5% CO₂ for 24 h. After the end of the culture, 50 μL of the supernatant was transferred to a new black ELISA plate, 50 μL/well of LDH detection substrate was added, the reaction was stopped after 10 min and LDH release was detected; the remaining cells in the wells were washed twice with 4% calf blood, incubated with 100 μg/mL human IgG for 10 min, and T cell were added to activate and detect antibodies (CD25-PE, CD4-APC, CD69-FITC, and CD8-APC) and incubating on ice for 20 min. The supernatant was washed and discarded, 60 μL/well PI was added, incubated on ice for 5 min, and detected by flow cytometer. FIGS. 10A and 10B show the killing of NCI-H929 cells and activation of T cells by the BCMA×CD3 κλ bispecific antibody, respectively. FIGS. 11A and 11B show the killing of RPMI-8226 cells and activation of T cells by the BCMA×CD3 κλ bispecific antibody, respectively. For tumor cells, NCI-H929 and RPMI-8226 with different expression levels of BCMA, the BCMA×CD3 κλ bispecific antibody can mediate effective killing of T cells, and the killing activity is equivalent to that of the control antibody.

### Example 6. Activation of the T Cell Activation Pathway by the BCMA×CD3 κλ Bispecific Antibody

The luciferase reporter plasmid pGL4.30[luc2P/NFAT-RE/Hygro] (Promega, E8481) was transfected into Jurkat cells and selected by adding 200 μg/ml hygromycin B to obtain Jurkat-NFAT luc stably transfected reporter cells. Jurkat-NFAT-luc reporter cells in the logarithmic growth phase and target cells RPMI-8226 were taken, centrifuged to discard the supernatant, and resuspended to 2×10⁶ cells/ml. 50 μl/well of the target cells were inoculated into a 96-well plate, the supernatant was discarded by centrifugation at 300 g for 5 min, and 50 μl/well of the Jurkat-NFAT-luc reporter cells were inoculated into a 96-well plate, and 50 μl of gradient diluted BCMA×CD3 κλ bispecific antibody or control antibody KLH×CD3 (initial concentration 20 μg/ml, 10-fold dilution for 10 gradients) was added to each well, and cultured at 37° C. for 6 h in 5% CO₂. After the end of the culture, 100 μL of detection reagent was added to each well according to ONE-Glo Luciferase Assay System instructions, allowed to stand at room temperature for 3 min, and detected on a microplate reader (Biotek Synergy HT). The detection results are shown in FIG. 12. The BCMA×CD3 κλ bispecific antibody can activate the NFAT signaling pathway of T cells when RPMI-8226 tumor cells are used as target cells; in the absence of target cells, the NFAT signaling pathway is not activated.

### Example 7. Non-Specific Activation of PBMC by BCMA×CD3 κλ Bispecific Antibody

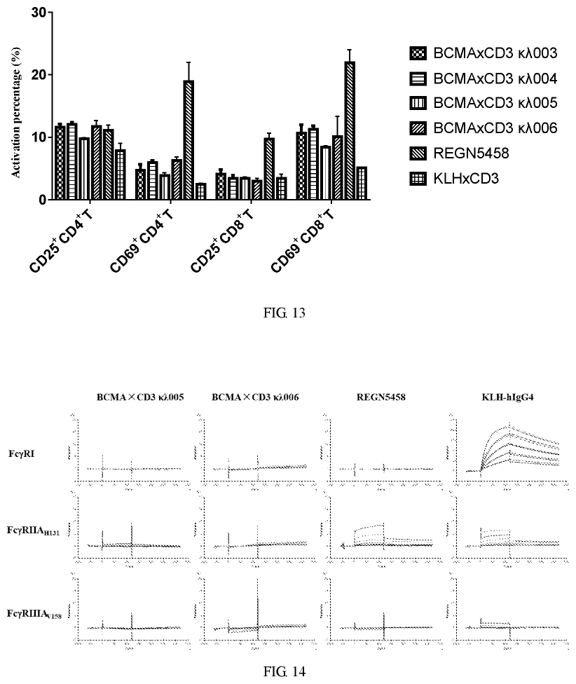

Freshly isolated PBMC was taken, and 50 μL of gradient diluted antibody (antibody concentration from 66.7 nM, 10-fold dilution for 7 gradients) was added to each well and cultured at 37° C. in 5% CO₂ for 24 h. After the end of the culture, 50 μL of the supernatant was transferred to a new black ELISA plate, 50 μL/well of LDH detection substrate was added, the reaction was stopped after 10 min and LDH release was detected; the remaining cells in the wells were washed twice with 4% calf blood, incubated with 100 μg/mL human IgG for 10 min, and T cell were added to activate and detect antibodies (CD25-PE, CD4-APC, CD69-FITC, and CD8-APC) and incubating on ice for 20 min. The supernatant was washed and discarded, 60 μL/well PI was added, incubated on ice for 5 min, and detected by flow cytometer. The results are shown in FIG. 13, in the absence of target cells, the BCMA×CD3 κλ bispecific antibody had no activation of peripheral blood T cells, comparable to the negative control KLH×CD3.

### Example 8. Binding of BCMA×CD3 κλ Bispecific Antibodies to Fc Receptors

50 Mg/ml His-Tag antibody was amino-coupled to CM5 chip to capture FcγRI, FcγRIIA_{H131}, and FcγRIIIA_{V158} recombinant proteins with His6 tag, respectively, with capture time of 40 s and flow rate of 10 μL/min, after baseline stable, gradient diluted antibody (initial concentration 37.5 μg/mL, 2-fold dilution) flowed through the chip with a flow rate of 30 μL/min, binding time of 120 s, dissociation time of 200 s, and affinity constant was obtained by fitting with Biacore evaluation software. The results are shown in FIG. 14. The BCMA×CD3 κλ bispecific antibody did not bind to activated receptors FcγRI, FcRIIA_{H131}, and FcγRIIIA_{V158}; wild-type IgG4 and control antibodies bound to FcγRI, FcγRIIA_{H131} or FcγRIIIA_{V158}, respectively.

### Example 9. Blocking of BCMA Receptor Binding to a Lgand by BCMA×CD3 I Bispecific Antibody

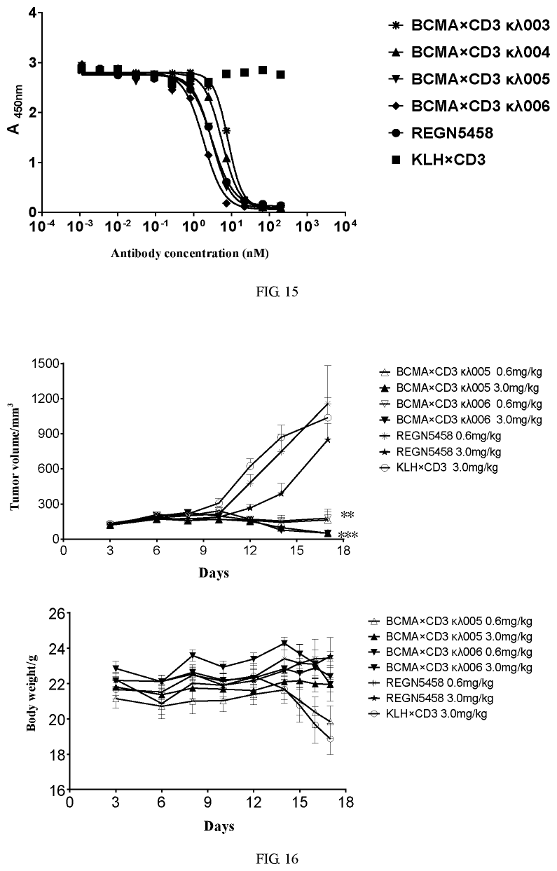

1 μg/mL human BCMA recombinant antigen was prepared with pH 9.6 carbonic acid buffer, added to a maxisorp96-well plate, and coated overnight at 4° C. After blocking with 5% skim milk/PBS for 1 h at room temperature, 50 μL antibody per well (4-fold dilution from 30 μg/mL for a total of 10 gradients) was added and incubated for 30 min at room temperature. 50 μL of biotinylated human APRIL recombinant protein (Novoprotein, CU89) at a concentration of 240 ng/mL was added and incubation was continued for 30 min. After 3 washes with 0.05% PBST (PBS+0.05% Tween-20) and PBS respectively, 100 μL of SA-HRP (diluted 1:8000) was added to each well and incubated at room temperature for 45 min. Washing 3 times with 0.05% PBST and PBS respectively, 100 μL TMB color-developing solution was added into each well, after 10 min, 50 μL concentrated sulfuric acid was added into each well to stop the reaction, read OD450 nm by a microplate reader, and perform three-parameter fitting curve using Graphpad software. The results are shown in FIG. 15. The BCMA×CD3 κλ bispecific antibody effectively blocked the binding of BCMA to the receptor APRIL.

### Example 10. Immune Reconstruction of Subcutaneous NCI-H929 Transplanted Tumor Model in Mice

Six to eight-week-old female B-NGD mice (Bio-Tech Co. Ltd.) were subcutaneously inoculated with 2×10⁶ NCI-H929 cells (mixed with Matrigel at 1:1). When the tumors grew to 60 mm³, they were randomly divided into administration group 3.0 mg/kg, administration group 0.6 mg/kg, administration group 0.12 mg/kg and negative control group KLH×CD3 3 mg/kg. Each mouse received 1×10⁷ PBMC cells via tail vein injection. Three days later, the first dose was administered to the mouse once every 5 days for a total of 2 doses. The tumor volume and body weight of the mice were monitored every 2 days. At the end of the experiment, the mice were killed by cervical dislocation, and the tumors were collected and weighed. The results are shown in FIG. 16. The in vivo efficacy of BCMA×CD3 κλ bispecific antibody was dose-related. The tumor inhibition rates of low-dose, medium-dose, and high-dose were 55%, 95%, and 108% (BCMA×CD3 κλ005) and 46%, 94%, and 108% (BCMA×CD3 κλ006), respectively. The effect was significantly better than that of the control antibody. The tumor-bearing mice tolerated the above doses well, without weight loss and other adverse reactions.

1. A bispecific antibody or antigen-binding portion thereof comprising:
(a) a first antigen-binding portion or an antigen-binding fragment thereof bound to a BCMA antigen on the surface of a target cell, the first antigen-binding portion comprises a first heavy chain and a first light chain, the first antigen-binding portion comprises a first binding domain that binds to the first antigen, wherein the first binding domain comprises a heavy chain CDR selected from the amino acid sequences of SEQ ID NOs: 12, 13, 14, 26, 27, 28, 36, 37, 43, 44, 50, 55, 56, 57, 65, 66, 71, 72, 73, 147 and 186, or any variant thereof, and/or a light chain CDR selected from the amino acid sequences of SEQ ID Nos: 17, 18, 19, 22, 23, 31, 32, 33, 40, 47, 60, 61, 62, 76, 77, 78, 83, 84, 87, 88, 89, 92 and 144 or any variant; and
(b) a second antigen-binding portion or antigen-binding fragment thereof bound to a CD3 antigen on the surface of a T cell, the second antigen-binding portion comprises a second heavy chain and a second light chain, the second antigen-binding portion comprises a second binding domain that binds to a second antigen.

2. The bispecific antibody or antigen-binding portion thereof of claim 1, wherein the first binding domain comprises a heavy chain CDR1 selected from the amino acid sequences of SEQ ID NOs: 12, 26, 55, 65, 71 or any variant thereof, a heavy chain CDR2 selected from the amino acid sequences of SEQ ID NOs: 13, 27, 36, 43, 50, 56, 66, 72, 147, 186 or any variant thereof, a heavy chain CDR3 selected from the amino acid sequences of SEQ ID NOs: 14, 28, 37, 44, 57, 73 or any variant thereof; and/or the first binding domain comprises a light chain CDR1 selected from the amino acid sequences of SEQ ID NOs: 17, 22, 31, 47, 60, 76, 83, 87, 92, 144 or any variant thereof, a light chain CDR2 selected from the amino acid sequences of SEQ ID NOs: 18, 23, 32, 40, 61, 77, 84, 88 or any variant thereof, a light chain CDR3 selected from the amino acid sequences of SEQ ID NOs: 19, 33, 62, 78, 89 or any variant thereof.

3. The bispecific antibody or antigen-binding portion thereof of claim 2, wherein,
the heavy chain CDR1, CDR2, and CDR3 sequences of the first binding domain are selected from: first heavy chain CDR1, CDR2, and CDR3 sequences of the amino acid sequences of SEQ ID NOs: 12, 13 and 14, first heavy chain CDR1, CDR2, and CDR3 sequences of the amino acid sequences of SEQ ID NOs: 12, 186 and 14, first heavy chain CDR1, CDR2, and CDR3 sequences of the amino acid sequences of SEQ ID NOs: 26, 27 and 28, first heavy chain CDR1, CDR2, and CDR3 sequences of the amino acid sequences of SEQ ID NOs: 12, 36 and 37, first heavy chain CDR1, CDR2, and CDR3 sequences of the amino acid sequences of SEQ ID NOs: 12, 43 and 44, first heavy chain CDR1, CDR2, and CDR3 sequences of the amino acid sequences of SEQ ID NOs: 12, 50 and 14, first heavy chain CDR1, CDR2, and CDR3 sequences of the amino acid sequences of SEQ ID NOs: 55, 56 and 57, first heavy chain CDR1, CDR2, and CDR3 sequences of the amino acid sequences of SEQ ID NOs: 65, 66 and 14, first heavy chain CDR1, CDR2, and CDR3 sequences of the amino acid sequences of SEQ ID NOs: 71, 72 and 73; first heavy chain CDR1, CDR2, and CDR3 sequences of the amino acid sequences of SEQ ID NOs: 71, 147 and 73; and the light chain CDR1, CDR2, and CDR3 sequences of the first binding domain are selected from: first light chain CDR1, CDR2 and CDR3 sequences of the amino acid sequences of SEQ ID NOs: 17, 18 and 19, first light chain CDR1, CDR2 and CDR3 sequences of the amino acid sequences of SEQ ID NOs: 22, 23 and 19, first light chain CDR1, CDR2 and CDR3 sequences of the amino acid sequences of SEQ ID NOs: 31, 32 and 33, first light chain CDR1, CDR2 and CDR3 sequences of the amino acid sequences of SEQ ID NOs: 17, 40 and 19, first light chain CDR1, CDR2 and CDR3 sequences of the amino acid sequences of SEQ ID NOs: 47, 18 and 19, first light chain CDR1, CDR2 and CDR3 sequences of the amino acid sequences of SEQ ID NOs: 60, 61 and 62, first light chain CDR1, CDR2 and CDR3 sequences of the amino acid sequences of SEQ ID NOs: 76, 77 and 78, first light chain CDR1, CDR2 and CDR3 sequences of the amino acid sequences of SEQ ID NOs: 83, 84 and 19, first light chain CDR1, CDR2, and CDR3 sequences of the amino acid sequences of SEQ ID NOs: 87, 88 and 89, first light chain CDR1, CDR2, and CDR3 sequences of the amino acid sequences of SEQ ID NOs: 92, 23 and 19, first light chain CDR1, CDR2, and CDR3 sequences of the amino acid sequences of SEQ ID NOs: 144, 77 and 78;
preferably, the first binding domain comprises first heavy chain CDR1, CDR2, and CDR3 sequences of the amino acid sequences of SEQ ID NOs: 12, 13 and 14, and first light chain CDR1, CDR2, and CDR3 sequences of the amino acid sequences of SEQ ID NOs: 17, 18 and 19;
preferably, the first binding domain comprises first heavy chain CDR1, CDR2, and CDR3 sequences of the amino acid sequences of SEQ ID NOs: 12, 13 and 14 and first light chain CDR1, CDR2, and CDR3 sequences of the amino acid sequences of SEQ ID NOs: 22, 23 and 19;
preferably, the first binding domain comprises first heavy chain CDR1, CDR2, and CDR3 sequences of the amino acid sequences of SEQ ID NOs: 26, 27, and 28 and first light chain CDR1, CDR2, and CDR3 sequences of the amino acid sequences of SEQ ID NOs: 31, 32, and 33;
preferably, the first binding domain comprises first heavy chain CDR1, CDR2, and CDR3 sequences of the amino acid sequences of SEQ ID NOs: 12, 36 and 37 and first light chain CDR1, CDR2, and CDR3 sequences of the amino acid sequences of SEQ ID NOs: 17, 40 and 19;
preferably, the first binding domain comprises first heavy chain CDR1, CDR2, and CDR3 sequences of the amino acid sequences of SEQ ID NOs: 12, 43 and 44 and first light chain CDR1, CDR2, and CDR3 sequences of the amino acid sequences of SEQ ID NOs: 47, 18 and 19;
preferably, the first binding domain comprises first heavy chain CDR1, CDR2, and CDR3 sequences of the amino acid sequences of SEQ ID NOs: 12, 50 and 14 and first light chain CDR1, CDR2, and CDR3 sequences of the amino acid sequences of SEQ ID NOs: 17, 40 and 19;
preferably, the first binding domain comprises first heavy chain CDR1, CDR2, and CDR3 sequences of the amino acid sequences of SEQ ID NOs: 55, 56 and 57 and first light chain CDR1, CDR2, and CDR3 sequences of the amino acid sequences of SEQ ID NOs: 60, 61 and 62;
preferably, the first binding domain comprises first heavy chain CDR1, CDR2, and CDR3 sequences of the amino acid sequences of SEQ ID NOs: 12, 13 and 14 and first light chain CDR1, CDR2, and CDR3 sequences of the amino acid sequences of SEQ ID NOs: 17, 40 and 19;
preferably, the first binding domain comprises first heavy chain CDR1, CDR2, and CDR3 sequences of the amino acid sequences of SEQ ID NOs: 65, 66 and 14 and first light chain CDR1, CDR2, and CDR3 sequences of the amino acid sequences of SEQ ID NOs: 17, 40 and 19;
preferably, the first binding domain comprises first heavy chain CDR1, CDR2, and CDR3 sequences of the amino acid sequences of SEQ ID NOs: 71, 72, and 73 and first light chain CDR1, CDR2, and CDR3 sequences of the amino acid sequences of SEQ ID NOs: 76, 77, and 78;
preferably, the first binding domain comprises first heavy chain CDR1, CDR2, and CDR3 sequences of the amino acid sequences of SEQ ID NOs: 12, 13 and 14 and first light chain CDR1, CDR2, and CDR3 sequences of the amino acid sequences of SEQ ID NOs: 83, 84 and 19;
preferably, the first binding domain comprises first heavy chain CDR1, CDR2, and CDR3 sequences of the amino acid sequences of SEQ ID NOs: 12, 13 and 14 and first light chain CDR1, CDR2, and CDR3 sequences of the amino acid sequences of SEQ ID NOs: 87, 88 and 89;
preferably, the first binding domain comprises first heavy chain CDR1, CDR2, and CDR3 sequences of the amino acid sequences of SEQ ID NOs: 12, 13 and 14 and first light chain CDR1, CDR2, and CDR3 sequences of the amino acid sequences of SEQ ID NOs: 92, 23 and 19;
preferably, the first binding domain comprises first heavy chain CDR1, CDR2, and CDR3 sequences of the amino acid sequences of SEQ ID NOs: 71, 147, 73 and first light chain CDR1, CDR2, and CDR3 sequences of the amino acid sequences of SEQ ID NOs: 144, 77, 78; preferably, the first binding domain comprises first heavy chain CDR1, CDR2, and CDR3 sequences of the amino acid sequences of SEQ ID NOs: 12, 186 and 14 and first light chain CDR1, CDR2, and CDR3 sequences of the amino acid sequences of SEQ ID NOs: 17, 40 and 19; preferably, the first binding domain comprises first heavy chain CDR1, CDR2, and CDR3 sequences of the amino acid sequences of SEQ ID NOs: 12, 186 and 14 and first light chain CDR1, CDR2, and CDR3 sequences of the amino acid sequences of SEQ ID NOs: 22, 23 and 19.

4. The bispecific antibody or antigen-binding portion thereof of claim 1, wherein the first binding domain comprises a first heavy chain variable region selected from the amino acid sequences of SEQ ID NOs: 10, 24, 34, 41, 48, 53, 63, 69, 79, 145, 150, 154, 158, 162, 166, 170, and 187, or any variant thereof, and/or comprises a first light chain variable region selected from the amino acid sequences of SEQ ID NOs: 15, 20, 29, 38, 45, 51, 58, 67, 74, 81, 85, 90, 142, 148, 152, 156, 160, 164, 168, and 188, or any variant thereof;
preferably, the first binding domain comprises a first heavy chain variable region of the amino acid sequence of SEQ ID NO: 10 or any variant thereof, and a first light chain variable region of the amino acid sequence of SEQ ID NO: 15 or any variant thereof;
preferably, the first binding domain comprises a first heavy chain variable region of the amino acid sequence of SEQ ID NO: 10 or any variant thereof, and a first light chain variable region of the amino acid sequence of SEQ ID NO: 20 or any variant thereof;
preferably, the first binding domain comprises a first heavy chain variable region of the amino acid sequence of SEQ ID NO: 24 or any variant thereof, and a first light chain variable region of the amino acid sequence of SEQ ID NO: 29 or any variant thereof;
preferably, the first binding domain comprises a first heavy chain variable region of the amino acid sequence of SEQ ID NO: 34 or any variant thereof, and a first light chain variable region of the amino acid sequence of SEQ ID NO: 38 or any variant thereof;
preferably, the first binding domain comprises a first heavy chain variable region of the amino acid sequence of SEQ ID NO: 41 or any variant thereof, and a first light chain variable region of the amino acid sequence of SEQ ID NO: 45 or any variant thereof;
preferably, the first binding domain comprises a first heavy chain variable region of the amino acid sequence of SEQ ID NO: 48 or any variant thereof, and a first light chain variable region of the amino acid sequence of SEQ ID NO: 51 or any variant thereof;
preferably, the first binding domain comprises a first heavy chain variable region of the amino acid sequence of SEQ ID NO: 53 or any variant thereof, and a first light chain variable region of the amino acid sequence of SEQ ID NO: 58 or any variant thereof;
preferably, the first binding domain comprises a first heavy chain variable region of the amino acid sequence of SEQ ID NO: 10 or any variant thereof, and a first light chain variable region of the amino acid sequence of SEQ ID NO: 38 or any variant thereof;
preferably, the first binding domain comprises a first heavy chain variable region of the amino acid sequence of SEQ ID NO: 63 or any variant thereof, and a first light chain variable region of the amino acid sequence of SEQ ID NO: 67 or any variant thereof;
preferably, the first binding domain comprises a first heavy chain variable region of the amino acid sequence of SEQ ID NO: 69 or any variant thereof, and a first light chain variable region of the amino acid sequence of SEQ ID NO: 74 or any variant thereof;
preferably, the first binding domain comprises a first heavy chain variable region of the amino acid sequence of SEQ ID NO: 79 or any variant thereof, and a first light chain variable region of the amino acid sequence of SEQ ID NO: 81 or any variant thereof;
preferably, the first binding domain comprises a first heavy chain variable region of the amino acid sequence of SEQ ID NO: 10 or any variant thereof, and a first light chain variable region of the amino acid sequence of SEQ ID NO: 85 or any variant thereof;
preferably, the first binding domain comprises a first heavy chain variable region of the amino acid sequence of SEQ ID NO: 10 or any variant thereof, and a first light chain variable region of the amino acid sequence of SEQ ID NO: 90 or any variant thereof;
preferably, the first binding domain comprises a first heavy chain variable region of the amino acid sequence of SEQ ID NO: 145 or any variant thereof, and a first light chain variable region of the amino acid sequence of SEQ ID NO: 142 or any variant thereof;
preferably, the first binding domain comprises a first heavy chain variable region of the amino acid sequence of SEQ ID NO: 150 or any variant thereof, and a first light chain variable region of the amino acid sequence of SEQ ID NO: 148 or any variant thereof;
preferably, the first binding domain comprises a first heavy chain variable region of the amino acid sequence of SEQ ID NO: 154 or any variant thereof, and a first light chain variable region of the amino acid sequence of SEQ ID NO: 152 or any variant thereof;
preferably, the first binding domain comprises a first heavy chain variable region of the amino acid sequence of SEQ ID NO: 158 or any variant thereof, and a first light chain variable region of the amino acid sequence of SEQ ID NO: 156 or any variant thereof;
preferably, the first binding domain comprises a first heavy chain variable region of the amino acid sequence of SEQ ID NO: 162 or any variant thereof, and a first light chain variable region of the amino acid sequence of SEQ ID NO: 160 or any variant thereof;
preferably, the first binding domain comprises a first heavy chain variable region of the amino acid sequence of SEQ ID NO: 166 or any variant thereof, and a first light chain variable region of the amino acid sequence of SEQ ID NO: 164 or any variant thereof;
preferably, the first binding domain comprises a first heavy chain variable region of the amino acid sequence of SEQ ID NO: 170 or any variant thereof, and a first light chain variable region of the amino acid sequence of SEQ ID NO: 168 or any variant thereof;
preferably, the first binding domain comprises a first heavy chain variable region of the amino acid sequence of SEO ID NO: 187 or any variant thereof, and a first light chain variable region of the amino acid sequence of SEO ID NO: 188 or any variant thereof.

5. The bispecific antibody or antigen-binding portion thereof of claim 1, wherein, the second binding domain comprises a heavy chain CDR selected from the amino acid sequences of SEQ ID NOs: 114, 115, 116, 119, 122, 125, 128, 131, 134, 135 or any variant thereof; and/or a light chain CDR selected from the amino acid sequences of SEQ ID NOs: 95, 96, 97, 102, 103, 108, 111 or any variant thereof;
preferably, the second binding domain comprises a second heavy chain CDR1 selected from the amino acid sequences of SEQ ID NOs: 114, 119, 134 or any variant thereof, a second heavy chain CDR2 selected from the amino acid sequences of SEQ ID NOs: 115, 135 or any variant thereof, a second heavy chain CDR3 selected from the amino acid sequences of SEQ ID NOs: 116, 122, 125, 128, 131 or any variant thereof, and/or a second light chain CDR1 selected from the amino acid sequences of SEQ ID NOs: 95, 102 or any variant thereof, a second light chain CDR2 selected from the amino acid sequences of SEQ ID NOs: 96, 103, 111 or any variant thereof, a second light chain CDR3 selected from the amino acid sequences of SEQ ID NOs: 91, 108 or any variant thereof;
preferably, the heavy chain CDR1, CDR2, and CDR3 sequences of the second binding domain are selected from: second heavy chain CDR1, CDR2, and CDR3 sequences of the amino acid sequences of SEQ ID NOs: 114, 115 and 116, second heavy chain CDR1, CDR2, and CDR3 sequences of the amino acid sequences of SEQ ID NOs: 119, 115 and 116, second heavy chain CDR1, CDR2, and CDR3 sequences of the amino acid sequences of SEQ ID NOs: 119, 115 and 122, second heavy chain CDR1, CDR2, and CDR3 sequences of the amino acid sequences of SEQ ID NOs: 119, 115 and 125, second heavy chain CDR1, CDR2, and CDR3 sequences of the amino acid sequences of SEQ ID NOs: 119, 115 and 128, second heavy chain CDR1, CDR2, and CDR3 sequences of the amino acid sequences of SEQ ID NOs: 119, 115 and 131, second heavy chain CDR1, CDR2, and CDR3 sequences of the amino acid sequences of SEQ ID NOs: 134, 135 and 116; and the light chain CDR1, CDR2, and CDR3 sequences of the second binding domain are selected from: second light chain CDR1, CDR2, and CDR3 sequences of the amino acid sequences of SEQ ID NOs: 95, 96 and 97, second light chain CDR1, CDR2, and CDR3 sequences of the amino acid sequences of SEQ ID NOs: 102, 103 and 97, second light chain CDR1, CDR2, and CDR3 sequences of the amino acid sequences of SEQ ID NOs: 95, 96 and 108, second light chain CDR1, CDR2, and CDR3 sequences of the amino acid sequences of SEQ ID NOs: 95, 111 and 108;
more preferably, the second binding domain comprises second heavy chain CDR1, CDR2, and CDR3 sequences of the amino acid sequences of SEQ ID NOs: 114, 115, 116 and second light chain CDR1, CDR2, and CDR3 sequences of the amino acid sequences of SEQ ID NOs: 95, 96, 97;
more preferably, the second binding domain comprises second heavy chain CDR1, CDR2, and CDR3 sequences of the amino acid sequences of SEQ ID NOs: 114, 115, 116 and second light chain CDR1, CDR2, and CDR3 sequences of the amino acid sequences of SEQ ID NOs: 102, 103, 97;
more preferably, the second binding domain comprises second heavy chain CDR1, CDR2, and CDR3 sequences of the amino acid sequences of SEQ ID NOs: 114, 115, 116 and second light chain CDR1, CDR2, and CDR3 sequences of the amino acid sequences of SEQ ID NOs: 95, 96, and 108;
more preferably, the second binding domain comprises second heavy chain CDR1, CDR2, and CDR3 sequences of the amino acid sequences of SEQ ID NOs: 114, 115, 116 and second light chain CDR1, CDR2, and CDR3 sequences of the amino acid sequences of SEQ ID NOs: 95, 111, and 108;
preferably, the second binding domain comprises a second heavy chain variable region selected from the amino acid sequences of SEQ ID NOs: 112, 117, 120, 123, 126, 129, 132, 136, 138, 140 or any variant thereof, and/or comprises a second light chain variable region selected from the amino acid sequences of SEQ ID NOs: 93, 98, 100, 104, 106, 109 or any variant thereof;
more preferably, the second binding domain comprises a second heavy chain variable region of the amino acid sequence of SEQ ID NO: 136 or any variant thereof, and a second light chain variable region of the amino acid sequence of SEQ ID NO: 93 or any variant thereof;
more preferably, the second binding domain comprises a second heavy chain variable region of the amino acid sequence of SEQ ID NO: 138 or any variant thereof, and a second light chain variable region of the amino acid sequence of SEQ ID NO: 93 or any variant thereof;
more preferably, the second binding domain comprises a second heavy chain variable region of the amino acid sequence of SEQ ID NO: 138 or any variant thereof, and a second light chain variable region of the amino acid sequence of SEQ ID NO: 98 or any variant thereof;
more preferably, the second binding domain comprises a second heavy chain variable region of the amino acid sequence of SEQ ID NO: 138 or any variant thereof, and a second light chain variable region of the amino acid sequence of SEQ ID NO: 100 or any variant thereof;
more preferably, the second binding domain comprises a second heavy chain variable region of the amino acid sequence of SEQ ID NO: 112 or any variant thereof, and a second light chain variable region of the amino acid sequence of SEQ ID NO: 106 or any variant thereof;
more preferably, the second binding domain comprises a second heavy chain variable region of the amino acid sequence of SEQ ID NO: 136 or any variant thereof, and a second light chain variable region of the amino acid sequence of SEQ ID NO: 106 or any variant thereof;
more preferably, the second binding domain comprises a second heavy chain variable region of the amino acid sequence of SEQ ID NO: 138 or any variant thereof, and a second light chain variable region of the amino acid sequence of SEQ ID NO: 106 or any variant thereof.

6. The bispecific antibody or antigen-binding portion thereof of claim 1, wherein, the first light chain of the first antigen-binding portion is a κ type light chain, the second light chain of the second antigen-binding portion is a λ type light chain,
preferably, the second light chain variable region of the second antigen-binding portion has a Gln₄₀Glu mutation (Vλ_{CD3}:Gln₄₀Glu); the second heavy chain variable region of the second antigen-binding portion has a Gln₃₉Lys mutation (VH_{CD3}:Gln₃₉Lys);
the first light chain variable region of the first antigen-binding portion has a Gln₄₂Lys mutation (VK_{BCMA}: Gln₄₂Lys); more preferably, the first heavy chain variable region of the first antigen-binding portion has a Gln₃₉Glu mutation (VH_{BCMA}: Gln₃₉Glu);
preferably, the Fc portion of the first antigen-binding portion and the second antigen-binding portion of the bispecific antibody adopts a knob-into-hole structure; preferably, a human IgG4 knob-into-hole structure is adopted.

7. The bispecific antibody or antigen-binding portion thereof of claim 1, wherein the first heavy chain comprises a heavy chain selected from SEQ ID NOs: 174, 178 or any variant thereof, the first light chain comprises a light chain selected from SEQ ID NOs: 172, 176 or any variant thereof;
preferably, the first heavy chain comprises a heavy chain selected from SEQ ID NO: 174 or any variant thereof, the first light chain comprises a light chain selected from SEQ ID NO: 172 or any variant thereof;
preferably, the first heavy chain comprises a heavy chain selected from SEQ ID NO: 174 or any variant thereof, the first light chain comprises a light chain selected from SEQ ID NO: 176 or any variant thereof;
preferably, the first heavy chain comprises a heavy chain selected from SEQ ID NO: 178 or any variant thereof, the first light chain comprises a light chain selected from SEQ ID NO: 172 or any variant thereof;
preferably, the first heavy chain comprises a heavy chain selected from SEQ ID NO: 178 or any variant thereof, the first light chain comprises a light chain selected from SEQ ID NO: 176 or any variant thereof.

8. The bispecific antibody or antigen-binding portion thereof of claim 1, wherein the second binding domain comprises a second heavy chain variable region of the amino acid sequence of SEQ ID NO: 138 or any variant thereof, and a second light chain variable region of the amino acid sequence of SEQ ID NO: 106 or any variant thereof;
preferably, the second heavy chain comprises a heavy chain of SEQ ID NO: 182 or any variant thereof, the second light chain comprises a light chain of SEQ ID NO: 180 or any variant thereof;
preferably, the first binding domain of the bispecific antibody comprises a first heavy chain variable region of the amino acid sequence of SEQ ID NO: 145 or any variant thereof, and a first light chain variable region of the amino acid sequence of SEQ ID NO: 142 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof;
preferably, the first binding domain of the bispecific antibody comprises a first heavy chain variable region of the amino acid sequence of SEQ ID NO: 150 or any variant thereof, and a first light chain variable region of the amino acid sequence of SEQ ID NO: 148 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof;
preferably, the first binding domain of the bispecific antibody comprises a first heavy chain variable region of the amino acid sequence of SEQ ID NO: 154 or any variant thereof, and a first light chain variable region of the amino acid sequence of SEQ ID NO: 152 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof;
preferably, the first binding domain of the bispecific antibody comprises a first heavy chain variable region of the amino acid sequence of SEQ ID NO: 158 or any variant thereof, and a first light chain variable region of the amino acid sequence of SEQ ID NO: 156 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof;
preferably, the first binding domain of the bispecific antibody comprises a first heavy chain variable region of the amino acid sequence of SEQ ID NO: 162 or any variant thereof, and a first light chain variable region of the amino acid sequence of SEQ ID NO: 160 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof;
preferably, the first binding domain of the bispecific antibody comprises a first heavy chain variable region of the amino acid sequence of SEQ ID NO: 166 or any variant thereof, and a first light chain variable region of the amino acid sequence of SEQ ID NO: 164 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof;
preferably, the first binding domain of the bispecific antibody comprises a first heavy chain variable region of the amino acid sequence of SEQ ID NO: 170 or any variant thereof, and a first light chain variable region of the amino acid sequence of SEQ ID NO: 168 or any variant thereof; the second binding domain comprises a second heavy chain variable region of the amino acid sequence of SEQ ID NO: 138 or any variant thereof, and a second light chain variable region of the amino acid sequence of SEQ ID NO: 106 or any variant thereof;
more preferably, the first heavy chain of the bispecific antibody comprises a heavy chain of SEQ ID NO: 174 or any variant thereof, the first light chain comprises a light chain of SEQ ID NO: 172 or any variant thereof; the second heavy chain comprises a heavy chain of SEQ ID NO: 182 or any variant thereof, the second light chain comprises a light chain of SEQ ID NO: 180 or any variant thereof;
more preferably, the first heavy chain of the bispecific antibody comprises a heavy chain of SEQ ID NO: 174 or any variant thereof, the first light chain comprises a light chain of SEQ ID NO: 176 or any variant thereof; the second heavy chain comprises a heavy chain of SEQ ID NO: 182 or any variant thereof, the second light chain comprises a light chain of SEQ ID NO: 180 or any variant thereof;
more preferably, the first heavy chain of the bispecific antibody comprises a heavy chain of SEQ ID NO: 178 or any variant thereof, the first light chain comprises a light chain of SEQ ID NO: 172 or any variant thereof; the second heavy chain comprises a heavy chain of SEQ ID NO: 182 or any variant thereof, the second light chain comprises a light chain of SEQ ID NO: 180 or any variant thereof;
more preferably, the first heavy chain of the bispecific antibody comprises a heavy chain of SEQ ID NO: 178 or any variant thereof, the first light chain comprises a light chain of SEQ ID NO: 176 or any variant thereof; the second heavy chain comprises a heavy chain of SEQ ID NO: 182 or any variant thereof, the second light chain comprises a light chain of SEQ ID NO: 180 or any variant thereof.

9. A BCMA-binding antibody or antigen-binding portion thereof, the antibody comprises the first antigen-binding portion of claim 1.

10. A nucleic acid encoding the bispecific antibody or antigen-binding portion thereof of claim 1 or a BCMA-binding antibody or antigen-binding portion thereof comprising the first antigen-binding portion;
preferably, the nucleic acid encoding the first heavy chain variable region of the first antigen-binding portion is selected from the nucleotide sequences of SEQ ID NOs: 11, 25, 35, 42, 49, 54, 64, 70, 80, 146, 151, 155, 159, 163, 167, 171, and 189, or any variant thereof; and/or the nucleic acid encoding the first light chain variable region of the first antigen-binding portion is selected from the nucleotide sequences of SEQ ID NOs: 16, 21, 30, 39, 46, 52, 59, 68, 75, 82, 86, 91, 143, 149, 153, 157, 161, 165, 169, and 190, or any variant thereof;
more preferably, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 16;
more preferably, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 21;
more preferably, the nucleic acid encoding the first heavy chain variable region of the first antigen-binding portion is the nucleotide sequence of SEQ ID NO: 25; and the nucleic acid encoding the first light chain variable region is the nucleotide sequence of SEQ ID NO: 30;
more preferably, the nucleic acid encoding the first heavy chain variable region of the first antigen-binding portion is the nucleotide sequence of SEQ ID NO: 35; and the nucleic acid encoding the first light chain variable region is the nucleotide sequence of SEQ ID NO: 39;
more preferably, the nucleic acid encoding the first heavy chain variable region of the first antigen-binding portion is the nucleotide sequence of SEQ ID NO: 42; and the nucleic acid encoding the first light chain variable region is the nucleotide sequence of SEQ ID NO: 46;
more preferably, the nucleic acid encoding the first heavy chain variable region of the first antigen-binding portion is the nucleotide sequence of SEQ ID NO: 49; and the nucleic acid encoding the first light chain variable region is the nucleotide sequence of SEQ ID NO: 52;
more preferably, the nucleic acid encoding the first heavy chain variable region of the first antigen-binding portion is the nucleotide sequence of SEQ ID NO: 54; and the nucleic acid encoding the first light chain variable region is the nucleotide sequence of SEQ ID NO: 59;
more preferably, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 39;
more preferably, the nucleic acid encoding the first heavy chain variable region of the first antigen-binding portion is the nucleotide sequence of SEQ ID NO: 64; and the nucleic acid encoding the first light chain variable region is the nucleotide sequence of SEQ ID NO: 68;
more preferably, the nucleic acid encoding the first heavy chain variable region of the first antigen-binding portion is the nucleotide sequence of SEQ ID NO: 70; and the nucleic acid encoding the first light chain variable region is the nucleotide sequence of SEQ ID NO: 75;
more preferably, the nucleic acid encoding the first heavy chain variable region of the first antigen-binding portion is the nucleotide sequence of SEQ ID NO: 80; and the nucleic acid encoding the first light chain variable region is the nucleotide sequence of SEQ ID NO: 82;
more preferably, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 86;
more preferably, the nucleic acid encoding the first heavy chain variable region of the first antigen-binding portion is the nucleotide sequence of SEQ ID NO: 11; and the nucleic acid encoding the first light chain variable region is the nucleotide sequence of SEQ ID NO: 91;
more preferably, the nucleic acid encoding the first heavy chain variable region of the first antigen-binding portion is the nucleotide sequence of SEQ ID NO: 146; and the nucleic acid encoding the first light chain variable region is the nucleotide sequence of SEQ ID NO: 143;
more preferably, the nucleic acid encoding the first heavy chain variable region of the first antigen-binding portion is the nucleotide sequence of SEQ ID NO: 151; and the nucleic acid encoding the first light chain variable region is the nucleotide sequence of SEQ ID NO: 149;
more preferably, the nucleic acid encoding the first heavy chain variable region of the first antigen-binding portion is the nucleotide sequence of SEQ ID NO: 155; and the nucleic acid encoding the first light chain variable region is the nucleotide sequence of SEQ ID NO: 153;
more preferably, the nucleic acid encoding the first heavy chain variable region of the first antigen-binding portion is the nucleotide sequence of SEQ ID NO: 159; and the nucleic acid encoding the first light chain variable region is the nucleotide sequence of SEQ ID NO: 157;
more preferably, the nucleic acid encoding the first heavy chain variable region of the first antigen-binding portion is the nucleotide sequence of SEQ ID NO: 163; and the nucleic acid encoding the first light chain variable region is the nucleotide sequence of SEQ ID NO: 161;
more preferably, the nucleic acid encoding the first heavy chain variable region of the first antigen-binding portion is the nucleotide sequence of SEQ ID NO: 167; and the nucleic acid encoding the first light chain variable region is the nucleotide sequence of SEQ ID NO: 165;
more preferably, the nucleic acid encoding the first heavy chain variable region of the first antigen-binding portion is the nucleotide sequence of SEQ ID NO: 171; and the nucleic acid encoding the first light chain variable region is the nucleotide sequence of SEQ ID NO: 169;
more preferably, the nucleic acid encoding the first heavy chain variable region of the first antigen-binding portion is the nucleotide sequence of SEO ID NO: 189; and the nucleic acid encoding the first light chain variable region is the nucleotide sequence of SEO ID NO: 190;
preferably, the nucleic acid encoding the first heavy chain of the first antigen-binding portion is selected from the nucleotide sequences of SEQ ID NOs: 175, 179 or any variant thereof; and/or
the nucleic acid encoding the first light chain of the first antigen-binding portion is selected from the nucleotide sequences of SEQ ID NOs: 173, 177 or any variant thereof;
more preferably, the nucleic acid encoding the first heavy chain of the first antigen-binding portion is the nucleotide sequence of SEQ ID NO: 175, and the nucleic acid encoding the first light chain of the first antigen-binding portion is the nucleotide sequence of SEQ ID NO: 173;
more preferably, the nucleic acid encoding the first heavy chain of the first antigen-binding portion is the nucleotide sequence of SEQ ID NO: 175, and the nucleic acid encoding the first light chain of the first antigen-binding portion is the nucleotide sequence of SEQ ID NO: 177;
more preferably, the nucleic acid encoding the first heavy chain of the first antigen-binding portion is the nucleotide sequence of SEQ ID NO: 179, and the nucleic acid encoding the first light chain of the first antigen-binding portion is the nucleotide sequence of SEQ ID NO: 173;
more preferably, the nucleic acid encoding the first heavy chain of the first antigen-binding portion is the nucleotide sequence of SEQ ID NO: 179, and the nucleic acid encoding the first light chain of the first antigen-binding portion is the nucleotide sequence of SEQ ID NO: 177;
preferably, the nucleic acid encoding the second heavy chain variable region of the second antigen-binding portion is selected from the nucleotide sequences of SEQ ID NOs: 113, 118, 121, 124, 127, 130, 133, 137, 139, 141 or any variant thereof; and/or the nucleic acid encoding the second light chain variable region of the second antigen-binding portion is selected from the nucleotide sequences of SEQ ID NOs: 94, 99, 101, 105, 107, 110 or any variant thereof;
more preferably, the nucleic acid encoding the second heavy chain variable region of the second antigen-binding portion is the nucleotide sequence of SEQ ID NO: 137; and the nucleic acid encoding the second light chain variable region is the nucleotide sequence of SEQ ID NO: 94;
more preferably, the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 94;
more preferably, the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 99;
more preferably, the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 101;
more preferably, the nucleic acid encoding the second heavy chain variable region of the second antigen-binding portion is the nucleotide sequence of SEQ ID NO: 113; and the nucleic acid encoding the second light chain variable region is the nucleotide sequence of SEQ ID NO: 107;
more preferably, the nucleic acid encoding the second heavy chain variable region of the second antigen-binding portion is the nucleotide sequence of SEQ ID NO: 137; and the nucleic acid encoding the second light chain variable region is the nucleotide sequence of SEQ ID NO: 107;
more preferably, the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107;
preferably, the nucleic acid encoding the second heavy chain of the second antigen-binding portion is the nucleotide sequence of SEQ ID NO: 183 or any variant thereof, and/or the nucleic acid encoding the second light chain of the second antigen-binding portion is the nucleotide sequence of SEQ ID NO: 181 or any variant thereof;
more preferably, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181;
more preferably, the nucleic acid encoding the first heavy chain variable region of the first antigen-binding portion of the bispecific antibody is the nucleotide sequence of SEQ ID NO: 146; and the nucleic acid encoding the first light chain variable region is the nucleotide sequence of SEQ ID NO: 143; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107;
more preferably, the nucleic acid encoding the first heavy chain variable region of the first antigen-binding portion of the bispecific antibody is the nucleotide sequence of SEQ ID NO: 151; and the nucleic acid encoding the first light chain variable region is the nucleotide sequence of SEQ ID NO: 149; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107;
more preferably, the nucleic acid encoding the first heavy chain variable region of the first antigen-binding portion of the bispecific antibody is the nucleotide sequence of SEQ ID NO: 155; and the nucleic acid encoding the first light chain variable region is the nucleotide sequence of SEQ ID NO: 153; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107;
more preferably, the nucleic acid encoding the first heavy chain variable region of the first antigen-binding portion of the bispecific antibody is the nucleotide sequence of SEQ ID NO: 159; and the nucleic acid encoding the first light chain variable region is the nucleotide sequence of SEQ ID NO: 157; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107;
more preferably, the nucleic acid encoding the first heavy chain variable region of the first antigen-binding portion of the bispecific antibody is the nucleotide sequence of SEQ ID NO: 163; and the nucleic acid encoding the first light chain variable region is the nucleotide sequence of SEQ ID NO: 161; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107;
more preferably, the nucleic acid encoding the first heavy chain variable region of the first antigen-binding portion of the bispecific antibody is the nucleotide sequence of SEQ ID NO: 167; and the nucleic acid encoding the first light chain variable region is the nucleotide sequence of SEQ ID NO: 165; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107;
more preferably, the nucleic acid encoding the first heavy chain variable region of the first antigen-binding portion of the bispecific antibody is the nucleotide sequence of SEQ ID NO: 171; and the nucleic acid encoding the first light chain variable region is the nucleotide sequence of SEQ ID NO: 169; the nucleic acid encoding the second heavy chain variable region of the second antigen-binding portion is the nucleotide sequence of SEQ ID NO: 139; and the nucleic acid encoding the second light chain variable region is the nucleotide sequence of SEQ ID NO: 107;
preferably, the nucleic acid encoding the first heavy chain of the first antigen-binding portion of the bispecific antibody is the nucleotide sequence of SEQ ID NO: 175, and the nucleic acid encoding the first light chain of the first antigen-binding portion is the nucleotide sequence of SEQ ID NO: 173, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181;
preferably, the nucleic acid encoding the first heavy chain of the first antigen-binding portion of the bispecific antibody is the nucleotide sequence of SEQ ID NO: 175, and the nucleic acid encoding the first light chain of the first antigen-binding portion is the nucleotide sequence of SEQ ID NO: 177, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181;
preferably, the nucleic acid encoding the first heavy chain of the first antigen-binding portion of the bispecific antibody is the nucleotide sequence of SEQ ID NO: 179, and the nucleic acid encoding the first light chain of the first antigen-binding portion is the nucleotide sequence of SEQ ID NO: 173, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181;
preferably, the nucleic acid encoding the first heavy chain of the first antigen-binding portion of the bispecific antibody is the nucleotide sequence of SEQ ID NO: 179, and the nucleic acid encoding the first light chain of the first antigen-binding portion is the nucleotide sequence of SEQ ID NO: 177, the nucleic acid encoding the second heavy chain of the second antigen-binding portion is the nucleotide sequence of SEQ ID NO: 183, and the nucleic acid encoding the second light chain of the second antigen-binding portion is the nucleotide sequence of SEQ ID NO: 181.

11. A vector comprising the nucleic acid of claim 10.

12. A cell comprising the nucleic acid of claim 10 or a vector comprising the nucleic acid.

13. A composition comprising the bispecific antibody or antigen-binding portion thereof of claim 1, a BCMA-binding antibody or antigen-binding portion thereof comprising the first antigen-binding portion, a nucleic acid encoding the bispecific antibody or antigen-binding portion thereof or a BCMA-binding antibody or antigen-binding portion thereof comprising the first antigen-binding portion, a vector comprising the nucleic acid and/or a cell comprising the nucleic acid or the vector.

14. An antibody-drug conjugate comprising the bispecific antibody or antigen-binding portion thereof of claim 1 or a BCMA-binding antibody or antigen-binding portion thereof comprising the first antigen-binding portion of claim 1 covalently attached to a therapeutic moiety;
preferably, the therapeutic moiety is selected from a cytotoxic moiety, a chemotherapeutic agent, a cytokine, an immunosuppressant, an immunostimulant, a lytic peptide, or a radioisotope;
preferably, the cytotoxic moiety is selected from paclitaxel; cytochalasin B; gramicidin D; ethidium bromide; emetine; mitomycin; etoposide; teniposide; vincristine; vinblastine; colchicine; doxorubicin; daunorubicin; dihydroxy anthracenedione; tubulin inhibitors such as maytansine or analogs or derivatives thereof, antimitotic agents such as monomethyl auristatin E or F or analogues or derivatives thereof, dolastatin 10 or 15 or an analogue thereof, irinotecan or an analog thereof, mitoxantrone; mithramycin; actinomycin D; 1-dehydrotestosterone; glucocorticoids; procaine; tetracaine; lidocaine; propranolol; puromycin; calicheamicin or an analogue or derivative thereof, antimetabolites such as methotrexate, 6 mercaptopurine, 6 thioguanine, cytarabine, fludarabine, 5 fluorouracil, decadiazine, hydroxyurea, asparaginase, gemcitabine or cladribine; alkylating agents such as mechlorethamine, thiopurine, chlorambucil, melphalan, carmustine (BSNU), lomustine (CCNU), cyclophosphamide, busulfan, dibromomannitol, streptozotocin, dacarbazine (DTIC), procarbazine, mitomycin C; platinum derivatives such as cisplatin or carboplatin; duocarmycin A, duocarmycin SA, rachelmycin (CC-1065) or analogs or derivatives thereof, antibiotics such as actinomycin, bleomycin, daunorubicin, doxorubicin, idarubicin, mithramycin, mitomycin, mitoxantrone, plicomycin, anthramycin (AMC); pyrrolo [2,1-c][1,4]-benzodiazepine (PDB); diphtheria toxin and related molecules such as diphtheria A chain and active fragments thereof and hybrid molecules, ricin toxin such as ricin A or deglycosylated ricin A chain toxin, cholera toxin, Shiga-like toxin such as SLT I, SLT II, SLT IIV, LT toxin, C3 toxin, Shiga toxin, pertussis toxin, tetanus toxin, soybean Bowman-Birk protease inhibitor, pseudomonas exotoxin, azlocillin, saporin, modeccin, gelsolin, abrin A chain, modeccin A chain, α-sarcin, Aleurites fordii proteins, dianthin proteins, Phytolacca americana proteins such as PAPI, PAPI, and PAP-S, Momordica charantia inhibitor, curcin, crotin, Sapaonaria officinalis inhibitor, gelonin, mitomycin, restrictocin, phenomycin, and enomycin toxins; ribonuclease (RNase); DNase I, staphylococcal endotoxin A; pokeweed antiviral protein; diphtheria toxin and pseudomonas endotoxin;
preferably, the cytokine is selected from IL-2, IL-4, IL-6, IL-7, IL-10, IL-12, IL-13, IL-15, IL-18, IL-23, IL-24, IL-27, IL-28a, IL-28b, IL-29, KGF, IFNα, IFNβ, IFNγ, GM-CSF, CD40L, Flt3 ligand, stem cell factor, ancestim, and TNFα;
preferably, the radioisotope is selected from ³, ¹⁴C, ¹⁵N, ³⁵S, ⁶⁷Cu, ⁹⁰Y, ⁹⁹Tc, ¹²⁵I, ¹³¹, ¹⁸⁶Re, ¹⁸⁸Re, ²¹¹At, ²¹²Bi, ²¹²Pb, ²¹³Bi, ²²⁵Ac, and ²²⁷Th.

15. A kit comprising the bispecific antibody or antigen-binding portion thereof of claim 1 or a BCMA-binding antibody or antigen-binding portion thereof comprising the first antigen-binding portion, or a nucleic acid encoding the bispecific antibody or antigen-binding portion thereof or the BCMA-binding antibody or antigen-binding portion thereof comprising the first antigen-binding portion, or a vector comprising the nucleic acid, or a cell comprising the nucleic acid or the vector.

16. A method of diagnosing, treating or preventing a disease associated with BCMA, wherein the method includes a step of: administering to a subject a therapeutically effective amount of the bispecific antibody or antigen-binding portion thereof of claim 1, a BCMA-binding antibody or antigen-binding portion thereof of comprising the first antigen-binding portion, or a nucleic acid molecule encoding the bispecific antibody or antigen-binding portion thereof or a BCMA-binding antibody or antigen-binding portion thereof comprising the first antigen-binding portion, or a vector comprising the nucleic acid, or a cell comprising the nucleic acid, or a composition, or an antibody-drug conjugate,
wherein the composition comprises the bispecific antibody or antigen-binding portion thereof, a BCMA-binding antibody or antigen-binding portion thereof comprising the first antigen-binding portion, a nucleic acid encoding the bispecific antibody or antigen-binding portion thereof or encoding a BCMA-binding antibody or antigen-binding portion thereof comprising the first antigen-binding portion, a vector comprising the nucleic acid and/or a cell comprising the nucleic acid or the vector,
wherein the antibody-drug conjugate comprises the bispecific antibody or antigen-binding portion thereof or a BCMA-binding antibody or antigen-binding portion thereof comprising the first antigen-binding portion covalently attached to a therapeutic moiety;
preferably, the disease associated with BCMA comprises B cell disease;
preferably, the disease is cancer; more preferably, the cancer is a B-cell related cancer selected from the group consisting of multiple myeloma, malignant plasmacytoma, hodgkin lymphoma, Nodular lymphocyte predominant Hodgkin lymphoma, kahler's disease and myeloid leukemia, plasma cell leukemia, plasmacytoma, B-cell prolymphocytic leukemia, hairy cell leukemia, B-cell non-Hodgkin lymphoma (NHL), acute myelogenous leukemia (AML), chronic lymphocytic leukemia (CLL), acute lymphocytic leukemia (ALL), chronic myelogenous leukemia (CML), follicular lymphoma, burkitt lymphoma, marginal zone lymphoma, mantle cell lymphoma, large cell lymphoma, precursor B-lymphocyte lymphoma, myeloid leukemia, waldenstrom's macroglobulinaemia, diffuse large B cell lymphoma, follicular lymphoma, marginal zone lymphoma, mucosa-associated lymphoid tissue lymphoma, small cell lymphocytic lymphoma, mantle cell lymphoma, burkitt lymphoma, primary mediastinal (thymic) large B cell lymphoma, lymphoplasmacytic lymphoma, waldenstrom's macroglobulinaemia, lymph node marginal zone B cell lymphoma, splenic marginal zone lymphoma, intravascular large B-cell lymphoma, primary effusion lymphoma, lymphomatoid granulomatosis, large B-cell lymphoma rich in T cells/histiocytes, primary central nervous system lymphoma, primary cutaneous diffuse large B-cell lymphoma (leg type), EBV-positive diffuse large B-cell lymphoma of the elderly, diffuse large B-cell lymphoma, intravascular large B-cell lymphoma associated with inflammation, ALK-positive large B-cell lymphoma, plasmablastic lymphoma, large B-cell lymphoma arising in HHV8-associated multicenter Castleman's disease, unclassified B-cell lymphoma with intermediate features between diffuse large B-cell lymphoma and Burkitt's lymphoma, unclassified B-cell lymphoma with intermediate features between diffuse large B-cell lymphoma and classic Hodgkin lymphoma, and other B-cell-related lymphomas;
more preferably, the B cell disease is a B cell disorder; preferably, the plasma cell disorder is selected from multiple myeloma, plasmacytoma, plasma cell leukemia, macroglobulinaemia, amyloidosis, waldenstrom's macroglobulinaemia, solitary plasmacytoma of the bone, extramedullary plasmacytoma, osteosclerotic myeloma, heavy chain disease, monoclonal gammopathy of indetermined significance, and multiple myeloma of stasis type;
the disease is an autoimmune disorder, such as systemic lupus erythematosus or rheumatoid arthritis.

17. A kit comprising the composition of claim 13.

18. A kit comprising the antibody-drug conjugate of claim 14.

19. The bispecific antibody or antigen-binding portion thereof of claim 1, wherein the first binding domain comprises a heavy chain variable region (VH) comprising VH CDR1, VH CDR2, and VH CDR3 that are shown in SEQ ID NOs: 174 or 178, and a light chain variable region (VL) comprising VL CDR1, VL CDR2, and VL CDR3 that are shown in SEQ ID NOs: 172 or 176;
preferably, the heavy chain comprises a heavy chain selected from SEQ ID NO: 174, the light chain comprises a light chain selected from SEQ ID NO: 172;
preferably, the heavy chain comprises a heavy chain selected from SEQ ID NO: 174, the light chain comprises a light chain selected from SEQ ID NO: 176;
preferably, the heavy chain comprises a heavy chain selected from SEQ ID NO: 178, the light chain comprises a light chain selected from SEQ ID NO: 172;
preferably, the heavy chain comprises a heavy chain selected from SEQ ID NO: 178, the light chain comprises a light chain selected from SEQ ID NO: 176.

20. A nucleic acid encoding the BCMA-binding antibody or antigen-binding fragment thereof of claim 19.

21. A vector comprising the nucleic acid of claim 20.

22. A cell comprising the nucleic acid of claim 20.

23. A vector comprising the vector of claim 21.

24. A composition comprising the antibody or antigen-binding portion thereof of claim 19, the nucleic acid encoding the BCMA-binding antibody or antigen-binding fragment thereof, the vector comprising the nucleic acid and/or the cell comprising the nucleic acid or the vector.

25. An antibody-drug conjugate comprising the bispecific antibody or antigen-binding portion thereof of claim 19 covalently attached to a therapeutic moiety.

26. A kit comprising the antibody or antigen-binding portion thereof of claim 19, the nucleic acid encoding the BCMA-binding antibody or antigen-binding fragment thereof, the vector comprising the nucleic acid, the cell comprising the nucleic acid or the vector, the composition comprising the antibody or antigen-binding portion thereof, the nucleic acid, the vector, the cell and/or the antibody-drug conjugate comprising the bispecific antibody or antigen-binding portion thereof of claim 19 covalently attached to a therapeutic moiety;
preferably, the therapeutic moiety is selected from a cytotoxic moiety, a chemotherapeutic agent, a cytokine, an immunosuppressant, an immunostimulant, a lytic peptide, or a radioisotope;
preferably, the cytotoxic moiety is selected from paclitaxel; cytochalasin B; gramicidin D; ethidium bromide; emetine; mitomycin; etoposide; teniposide; vincristine; vinblastine; colchicine; doxorubicin; daunorubicin; dihydroxy anthracenedione; tubulin inhibitors such as maytansine or analogs or derivatives thereof, antimitotic agents such as monomethyl auristatin E or F or analogues or derivatives thereof, dolastatin 10 or 15 or an analogue thereof, irinotecan or an analog thereof, mitoxantrone; mithramycin; actinomycin D; 1-dehydrotestosterone; glucocorticoids; procaine; tetracaine; lidocaine; propranolol; puromycin; calicheamicin or an analogue or derivative thereof, antimetabolites such as methotrexate, 6 mercaptopurine, 6 thioguanine, cytarabine, fludarabine, 5 fluorouracil, decadiazine, hydroxyurea, asparaginase, gemcitabine or cladribine; alkylating agents such as mechlorethamine, thiopurine, chlorambucil, melphalan, carmustine (BSNU), lomustine (CCNU), cyclophosphamide, busulfan, dibromomannitol, streptozotocin, dacarbazine (DTIC), procarbazine, mitomycin C; platinum derivatives such as cisplatin or carboplatin; duocarmycin A, duocarmycin SA, rachelmycin (CC-1065) or analogs or derivatives thereof, antibiotics such as actinomycin, bleomycin, daunorubicin, doxorubicin, idarubicin, mithramycin, mitomycin, mitoxantrone, plicomycin, anthramycin (AMC); pyrrolo [2,1-c][1,4]-benzodiazepine (PDB); diphtheria toxin and related molecules such as diphtheria A chain and active fragments thereof and hybrid molecules, ricin toxin such as ricin A or deglycosylated ricin A chain toxin, cholera toxin, Shiga-like toxin such as SLT I, SLT II, SLT IIV, LT toxin, C3 toxin, Shiga toxin, pertussis toxin, tetanus toxin, soybean Bowman-Birk protease inhibitor, pseudomonas exotoxin, azlocillin, saporin, modeccin, gelsolin, abrin A chain, modeccin A chain, α-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitomycin, restrictocin, phenomycin, and enomycin toxins; ribonuclease (RNase); DNase I, staphylococcal endotoxin A; pokeweed antiviral protein; diphtheria toxin and pseudomonas endotoxin;
preferably, the cytokine is selected from IL-2, IL-4, IL-6, IL-7, IL-10, IL-12, IL-13, IL-15, IL-18, IL-23, IL-24, IL-27, IL-28a, IL-28b, IL-29, KGF, IFNα, IFNβ, IFNγ, GM-CSF, CD40L, Flt3 ligand, stem cell factor, ancestim, and TNFα;
preferably, the radioisotope is selected from ³, ¹⁴C, ¹⁵N, ³⁵S, ⁶⁷Cu, ⁹⁰Y, ⁹⁹Tc, ¹²⁵L, ¹³¹, ¹⁸⁶Re, ¹⁸⁸Re, ²¹¹At, ²¹²Bi, ²¹²Pb, ²¹³Bi, ²²⁵Ac, and ²²⁷Th.

27. Use of the specific antibody or antigen-binding portion thereof of any of claim 19, the nucleic acid encoding the BCMA-binding antibody or antigen-binding fragment thereof, the vector comprising the nucleic acid, the cell comprising the nucleic acid or the vector, the composition comprising the antibody or antigen-binding portion thereof, the nucleic acid, the vector, the cell and/or the antibody-drug conjugate comprising the bispecific antibody or antigen-binding portion thereof of claim 19 covalently attached to a therapeutic moiety in diagnosing, treatment or prevention of a disease associated with BCMA;
preferably, the disease associated with BCMA comprises B cell disease;
preferably, the disease is cancer; more preferably, the cancer is a B-cell related cancer selected from the group consisting of multiple myeloma, malignant plasmacytoma, hodgkin lymphoma, Nodular lymphocyte predominant Hodgkin lymphoma, kahler's disease and myeloid leukemia, plasma cell leukemia, plasmacytoma, B-cell prolymphocytic leukemia, hairy cell leukemia, B-cell non-Hodgkin lymphoma (NHL), acute myelogenous leukemia (AML), chronic lymphocytic leukemia (CLL), acute lymphocytic leukemia (ALL), chronic myelogenous leukemia (CML), follicular lymphoma, burkitt lymphoma, marginal zone lymphoma, mantle cell lymphoma, large cell lymphoma, precursor B-lymphocyte lymphoma, myeloid leukemia, waldenstrom's macroglobulinaemia, diffuse large B cell lymphoma, follicular lymphoma, marginal zone lymphoma, mucosa-associated lymphoid tissue lymphoma, small cell lymphocytic lymphoma, mantle cell lymphoma, burkitt lymphoma, primary mediastinal (thymic) large B cell lymphoma, lymphoplasmacytic lymphoma, waldenstrom's macroglobulinaemia, lymph node marginal zone B cell lymphoma, splenic marginal zone lymphoma, intravascular large B-cell lymphoma, primary effusion lymphoma, lymphomatoid granulomatosis, large B-cell lymphoma rich in T cells/histiocytes, primary central nervous system lymphoma, primary cutaneous diffuse large B-cell lymphoma (leg type), EBV-positive diffuse large B-cell lymphoma of the elderly, diffuse large B-cell lymphoma, intravascular large B-cell lymphoma associated with inflammation, ALK-positive large B-cell lymphoma, plasmablastic lymphoma, large B-cell lymphoma arising in HHV8-associated multicenter Castleman's disease, unclassified B-cell lymphoma with intermediate features between diffuse large B-cell lymphoma and Burkitt's lymphoma, unclassified B-cell lymphoma with intermediate features between diffuse large B-cell lymphoma and classic Hodgkin lymphoma, and other B-cell-related lymphomas;
more preferably, the B cell disease is a B cell disorder; preferably, the plasma cell disorder is selected from multiple myeloma, plasmacytoma, plasma cell leukemia, macroglobulinaemia, amyloidosis, waldenstrom's macroglobulinaemia, solitary plasmacytoma of the bone, extramedullary plasmacytoma, osteosclerotic myeloma, heavy chain disease, monoclonal gammopathy of indetermined significance, and multiple myeloma of stasis type; or
the disease is an autoimmune disorder, such as systemic lupus erythematosus or rheumatoid arthritis.


SEQUENCE LISTING (185 sequences)

SEQ ID NO: 1 - protein, 290 aa, Homo sapiens
MLQMAGQCSQNEYFDSLLHACIPCQLRCSSNTPPLTCQRYCNASVTNSVKGTNASGAEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 290 aa, Macaca fascicularis
MLQMARQCSQNEYFDSLLHDCKPCQLRCSSTPPLTCQRYCNASMTNSVKGMNAASGAEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 304 aa, Homo sapiens
MLQMAGQCSQNEYFDSLLHACIPCQLRCSSNTPPLTCQRYCNASVTNSVKGTNASGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGSGLNDIFEAQKIEWHE

SEQ ID NO: 4 - protein, 303 aa, Macaca fascicularis
MLQMARQCSQNEYFDSLLHDCKPCQLRCSSTPPLTCQRYCNASMTNSVKGMNASGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGSGLNDIFEAQKIEWHE

SEQ ID NO: 5 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 306 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 7 - protein, 306 aa, Homo sapiens; identical to SEQ ID NO: 6

SEQ ID NO: 8 - protein, 305 aa, Macaca fascicularis
QSFEENRKLNVYNQEDGSVLLTCHVKNTNITWFKEGKMIDILTAHKNKWNLGSNTKDPRGVYQCKGSKDKSKTLQVYYRMCQNCIELNAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 330 aa, Macaca fascicularis
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMDAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGSGLNDIFEAQKIEWHE

SEQ ID NO: 10 - protein, 122 aa, Homo sapiens
EIQLVQSGPELKKPGETVKISCKASGYIFTNFGMNWVRQAPGKALKWMGWINTYTGEQIYADDFKGRFAFSLETSASTAYLQINNLKNEDTATYFCARGEIYYGYDVGFVYWGQGTLVTVSA

SEQ ID NO: 11 - DNA, 366 bp, Homo sapiens
gagatccagttggtgcagtctggacctgagctgaagaagcctggagagacagtcaagatctcctgcaaggcttctgggtatatcttcacaaactttggaatgaactgggtgaggcaggctccaggaaaggctttaaagtggatgggctggataaacacctacactggagaacaaatatatgctgatgacttcaagggacggtttgccttctctttggaaacctctgccagcactgcctatttgcagatcaacaacctcaaaaatgaggacacggctacatatttctgtgcaagaggggagatctactatggttacgacgtgggctttgtttactggggccaagggactctggtcactgtctctgca

SEQ ID NO: 12 - protein, 6 aa, Mus musculus
TNFGMN

SEQ ID NO: 13 - protein, 17 aa, Mus musculus
WINTYTGEQIYADDFKG

SEQ ID NO: 14 - protein, 13 aa, Mus musculus
GEIYYGYDVGFVY

SEQ ID NO: 15 - protein, 111 aa, Mus musculus
DIVLTQSPASLTVSLGQRATISCRASKSVSTSGYSYMHWYQQKPGQPPKLLIYLASDLESGVSARFSGSGSGTDFTLNIHPVEEEDAATYYCQHSRELPWTFGGGTKLEIK

SEQ ID NO: 16 - DNA, 333 bp, Mus musculus
gatattgtgctaactcagtctcctgcttccttaactgtatctctggggcagagggccaccatctcatgcagggccagcaaaagtgtcagtacatctggctatagttatatgcactggtaccaacagaaaccaggacagccacccaaactcctcatctatcttgcatccgacctagaatctggggtctctgccaggttcagtggcagtgggtctgggacagacttcaccctcaacatccatcctgtggaggaggaggatgctgcaacctattactgtcaacacagtagggaacttccgtggacgttcggtggaggcaccaagctggaaataaaa

SEQ ID NO: 17 - protein, 15 aa, Mus musculus
RASKSVSTSGYSYMH

SEQ ID NO: 18 - protein, 7 aa, Mus musculus
LASDLES

SEQ ID NO: 19 - protein, 9 aa, Mus musculus
QHSRELPWT

SEQ ID NO: 20 - protein, 111 aa, Mus musculus
DIVLTQSPASLAISLGQRATISCRASKSVTTSGYSYIHWYQQKPGQPPKLLIYLASDLEAGVPARFSGSGSGTDFTLNIHPVEEEDAATYYCQHSRELPWTFGGGTKLELK

SEQ ID NO: 21 - DNA, 333 bp, Mus musculus
gacattgtgctaacacagtctcctgcttccttagctatatctctggggcagagggccaccatctcatgcagggccagcaaaagtgtcactacatctggctatagttatatacactggtaccaacagaaaccaggacagccacccaaactcctcatctatcttgcatccgacctagaagctggggtccctgccaggttcagtggcagtgggtctgggacagacttcaccctcaacatccatcctgtggaggaagaggatgctgcaacctattactgtcagcacagtagggagcttccgtggacgttcggtggaggcaccaagctggagctgaaa

SEQ ID NO: 22 - protein, 15 aa, Mus musculus
RASKSVTTSGYSYIH

SEQ ID NO: 23 - protein, 7 aa, Mus musculus
LASDLEA

SEQ ID NO: 24 - protein, 118 aa, Mus musculus
QVQLQQSGPELVKPGASVNLSCKASGYTFTNYVIHWVKQKPGQGLEWIGYTIPYTDVTKYNEKFKAKATLTSDKSSSTAFMELSSLTSEDSAVYYCARYDFDGYFDYWGQGTTLTVSS

SEQ ID NO: 25 - DNA, 354 bp, Mus musculus
caggtccagctgcaacagtctggacctgagctggtgaagcctggggcttcagtgaatttgtcctgcaaggcttctggatacacattcactaattatgttatacactgggtaaagcagaagcctgggcagggccttgagtggattggatatactatcccttacactgatgttactaagtacaatgagaaattcaaagccaaggccacactgacttcagacaaatcctccagcacagccttcatggagctcagcagcctgacctctgaggactctgcggtctattactgtgcaagatatgatttcgacggctactttgactactggggccaaggcaccactctcacagtctcctca

SEQ ID NO: 26 - protein, 6 aa, Mus musculus
TNYVIH

SEQ ID NO: 27 - protein, 17 aa, Mus musculus
YTIPYTDVTKYNEKFKA

SEQ ID NO: 28 - protein, 9 aa, Mus musculus
YDFDGYFDY

SEQ ID NO: 29 - protein, 112 aa, Mus musculus
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLELK

SEQ ID NO: 30 - DNA, 336 bp, Mus musculus
gatattgtgatgactcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccgtggacgttcggtggaggcaccaagctggagctgaaa

SEQ ID NO: 31 - protein, 16 aa, Mus musculus
RSSKSLLHSNGITYLY

SEQ ID NO: 32 - protein, 7 aa, Mus musculus
QMSNLAS

SEQ ID NO: 33 - protein, 9 aa, Mus musculus
AQNLELPWT

SEQ ID NO: 34 - protein, 122 aa, Mus musculus
EVQLQQSGPELKKPGETVKISCKASGYTFTNFGMNWVRQTPGKALKWMGWINTYTGDLIYGDDFKGRFAFSLETSASTAYLQINNLKNEDTATYFCARGEIYYGYDVGFAYWGQGTLVTVSA

SEQ ID NO: 35 - DNA, 366 bp, Mus musculus
gaggtccagctgcagcagtctggacctgagctgaagaagcctggagagacagtcaagatctcctgcaaggcttctgggtataccttcacaaactttggaatgaactgggtgaggcagactccaggaaaggctttaaagtggatgggctggataaacacctacactggagacttaatatatggtgatgactttaagggacggtttgccttctctttggaaacctctgccagcactgcctatttacagatcaacaacctcaaaaatgaggacacggctacatatttctgtgcaagaggggagatctactatggttacgacgtggggtttgcttactggggccaagggactctggtcactgtctctgca

SEQ ID NO: 36 - protein, 17 aa, Mus musculus
WINTYTGDLIYGDDFKG

SEQ ID NO: 37 - protein, 13 aa, Mus musculus
GEIYYGYDVGFAY

SEQ ID NO: 38 - protein, 111 aa, Mus musculus
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPWTFGGGTKLEIK

SEQ ID NO: 39 - DNA, 333 bp, Mus musculus
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggtagtgggtctgggacagacttcaccctcaacatccatcctgtggaggaggaggatgctgcaacctattactgtcagcacagtagggagcttccgtggacgttcggtggaggcaccaagctggaaatcaaa

SEQ ID NO: 40 - protein, 7 aa, Mus musculus
LASNLES

SEQ ID NO: 41 - protein, 122 aa, Mus musculus
EIQLVQSGPELKKPGETVKISCKASGYIFTNFGMNWVRQTPGKALKWMGWINTYTGETIYADDFKGLFAFSLEVPASTAYLQINNLKNEDTATYFCARGEIYYGFDVGFAYWGQGTLVTVSA

SEQ ID NO: 42 - DNA, 366 bp, Mus musculus
gagatccagttggtgcagtctggacctgagctgaagaagcctggagagacagtcaagatctcctgcaaggcttctgggtatattttcacaaactttggaatgaactgggtgaggcagactccaggaaaggctttaaagtggatgggctggataaacacctacactggagaaacaatatatgctgatgacttcaagggactatttgccttctctttggaagtgcctgccagcactgcctatttgcagatcaacaacctcaaaaatgaggacacggctacatatttctgtgcaagaggggagatctactatggtttcgacgtgggatttgcttactggggccaagggactctggtcactgtctctgca

SEQ ID NO: 43 - protein, 17 aa, Mus musculus
WINTYTGETIYADDFKG

SEQ ID NO: 44 - protein, 13 aa, Mus musculus
GEIYYGFDVGFAY

SEQ ID NO: 45 - protein, 111 aa, Mus musculus
DIVLTQSPASLTVSLGQRATISCRASKSVSTSGYSYIHWYQQKPGQPPKLLIYLASDLESGVPARFSGSGSGTDFTLNIHPVEEEDAATYYCQHSRELPWTFGGGTKLELK

SEQ ID NO: 46 - DNA, 333 bp, Mus musculus
gacattgtgctaacacagtctcctgcttccttaactgtatctctggggcagagggccaccatctcatgcagggccagcaaaagtgtcagtacatctggctatagttatatacactggtaccaacagaaaccaggacagccacccaaactcctcatctatcttgcatccgacctagaatctggggtccctgccaggttcagtggcagtgggtctgggacagacttcaccctcaacatccatcctgtggaggaggaggatgctgcaacctattactgtcaacacagtagggaacttccgtggacgttcggtggaggcaccaagctggagctgaaa

SEQ ID NO: 47 - protein, 15 aa, Mus musculus
RASKSVSTSGYSYIH

SEQ ID NO: 48 - protein, 122 aa, Mus musculus
EIQLVQSGPELKKPGETVKISCTASGYTFTNFGMNWVRQAPGKALRWMGWINTYTGDQIYADDFKGRFAFSLETSASTAYLQINNLKNEDTATYFCARGEIYYGYDVGFVYWGQGTLVTVSA

SEQ ID NO: 49 - DNA, 366 bp, Mus musculus
gagatccagttggtgcagtctggacctgagctgaagaagcctggagagacagtcaagatctcctgcacggcttctgggtataccttcacaaactttggaatgaactgggtgaggcaggctccaggaaaggctttaaggtggatgggctggataaacacctacactggagaccaaatatatgctgatgacttcaagggacggtttgccttctctttggaaacctctgccagcactgcctatttgcagatcaacaacctcaaaaatgaggacacggctacatatttctgtgcaagaggggagatctactatggttacgacgtgggctttgtttattggggccaagggactctggtcactgtctctgca

SEQ ID NO: 50 - protein, 17 aa, Mus musculus
WINTYTGDQIYADDFKG

SEQ ID NO: 51 - protein, 111 aa, Mus musculus; identical to SEQ ID NO: 38

SEQ ID NO: 52 - DNA, 333 bp, Mus musculus
gacattgtgctaacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtggacgttcggtggaggcaccaagctggaaataaaa

SEQ ID NO: 53 - protein, 113 aa, Mus musculus
EVQLQQSGTVLTRPGASVKLSCKASGYSFSSYWMHWLKQRPGQGLEWIGAIYPGNSRTTYNQKFKGEAKLTADTSASTAYMDLSSLTNADSAVYFCTREETYWGQGTLVTVSA

SEQ ID NO: 54 - DNA, 339 bp, Mus musculus
gaggttcagctccagcagtctgggactgtgctgacaaggcctggggcttccgtgaagttgtcctgcaaggcttctggctacagcttttccagctactggatgcactggctaaaacagaggcctggacagggtctagaatggattggtgctatttatcctggaaatagtcgtactacctacaaccagaagttcaagggcgaggccaaactgactgcggacacttccgccagcactgcctacatggacctcagcagcctgacaaatgccgactctgcggtctatttctgtacaagagaggagacttactggggccaagggactctggtcactgtctctgca

SEQ ID NO: 55 - protein, 6 aa, Mus musculus
SSYWMH

SEQ ID NO: 56 - protein, 17 aa, Mus musculus
AIYPGNSRTTYNQKFKG

SEQ ID NO: 57 - protein, 6 aa, Mus musculus
TREETY

SEQ ID NO: 58 - protein, 112 aa, Mus musculus
DVVMTQTPLSLSVTIGQPASISCKSSQGLLDSYGVTYLNWLFQRPGQPPKRLIYLVSELDSGVPDRFTGSGSGTDFTLRISRVEAEDLGIYYCWQGSHFPLTFGAGTKLELK

SEQ ID NO: 59 - DNA, 336 bp, Mus musculus
gatgttgtgatgacccagactccactctctttgtcggttaccattggacaaccagcctccatctcttgcaagtcaagtcagggcctcttagatagttatggagtgacatatttgaattggttgtttcagaggccaggccagcctccaaagcgcctaatctatctagtgtctgaactggactctggagtccctgacaggttcactggcagtggatcagggacagatttcacactgagaatcagcagagtggaggctgaggatttgggaatttattattgctggcaaggttcacattttcctctcacgttcggtgctgggaccaagctggagctgaaa

SEQ ID NO: 60 - protein, 16 aa, Mus musculus
KSSQGLLDSYGVTYLN

SEQ ID NO: 61 - protein, 7 aa, Mus musculus
LVSELDS

SEQ ID NO: 62 - protein, 9 aa, Mus musculus
WQGSHFPLT

SEQ ID NO: 63 - protein, 122 aa, Mus musculus
EVQLQQSGPELRKPGETVKISCKASGYTFTNYGMNWVKQTPGKGLKWMGWINTYTGERIYADDFKGRFAFSLETSASTAYLQINNLKNEDTATYFCARGEIYYGYDVGFVYWGQGTLVTVSA

SEQ ID NO: 64 - DNA, 366 bp, Mus musculus
gaggtccagttgcagcagtctggacctgagctgaggaagcctggagagacagtcaagatctcctgcaaggcttctgggtataccttcacaaactatggaatgaactgggtgaagcagactccaggaaagggtttaaagtggatgggctggataaacacctacactggagaacgaatatatgctgatgacttcaagggacggtttgccttctctttggaaacctctgccagcactgcctatttgcagatcaacaacctcaaaaatgaggacacggctacatatttctgtgcaagaggggagatctactatggttacgacgtgggctttgtttactggggccaagggactctggtcactgtctctgca

SEQ ID NO: 65 - protein, 6 aa, Mus musculus
TNYGMN

SEQ ID NO: 66 - protein, 17 aa, Mus musculus
WINTYTGERIYADDFKG

SEQ ID NO: 67 - protein, 111 aa, Mus musculus
DIVLTQSPASLVVSLGQRATISCRASKSVSTSGYSYMHWYQQKPGQPPKLLIYLASNLESGVPARFSGSGSGTDFTLNIHPVEEEDAATYYCQHSRELPWTFGGGTRLEIK

SEQ ID NO: 68 - DNA, 333 bp, Mus musculus
gacattgtgctgacacagtctcctgcttccttagttgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtggacgttcggtggaggcaccagactggaaataaaa

SEQ ID NO: 69 - protein, 121 aa, Mus musculus
QVQLQQSGAELVRPGTSVKMSCKAAGYTFTNYWIGWVKQRPGHGLEWIGDIYPGSGDTNYNGKFKGKATLAADISSNAAYMQLSSLTSEDSAIYYCARSYYRNDAASFAYWGQGTLVTVSA

SEQ ID NO: 70 - DNA, 363 bp, Mus musculus
caggtccagctgcagcagtctggagctgagctggtaaggcctgggacttcagtgaagatgtcctgcaaggctgctggatacaccttcactaactactggataggttgggtaaagcagaggcctggacatggccttgagtggattggagatatttaccctggaagtggtgatactaattacaatgggaagttcaagggcaaggccacactggctgcagacatttcctccaacgcagcctacatgcagctcagcagcctgacatctgaggactctgccatctattactgtgcaagaagctactataggaacgacgcggcctcatttgcttactggggccaagggactctggtcactgtctctgca

SEQ ID NO: 71 - protein, 6 aa, Mus musculus
TNYWIG

SEQ ID NO: 72 - protein, 17 aa, Mus musculus
DIYPGSGDTNYNGKFKG

SEQ ID NO: 73 - protein, 12 aa, Mus musculus
SYYRNDAASFAY

SEQ ID NO: 74 - protein, 107 aa, Mus musculus
DIVMTQSHKFMSTSVGDRVNITCKASQDVDTAVAWYQQKPSQSPKLLIYWTSTRHTGVPDRFTGSGSGTDFTLTISNVQSEDLADYFCQQFSSYPLTFGAGTKLELK

SEQ ID NO: 75 - DNA, 321 bp, Mus musculus
gacattgtgatgacccagtctcacaaattcatgtccacatcagtaggagacagggtcaacatcacctgcaaggccagtcaggatgtggatactgctgtagcctggtatcaacagaaaccaagtcaatctcctaaactactgatttactggacatccacccggcacactggagtccctgatcgcttcacaggcagtggatctgggacagatttcactctcaccattagcaatgtgcagtctgaagacttggcagattatttctgtcaacaatttagtagctatcctctcacgttcggtgctgggaccaagctggagctgaaa

SEQ ID NO: 76 - protein, 11 aa, Mus musculus
KASQDVDTAVA

SEQ ID NO: 77 - protein, 7 aa, Mus musculus
WTSTRHT

SEQ ID NO: 78 - protein, 9 aa, Mus musculus
QQFSSYPLT

SEQ ID NO: 79 - protein, 122 aa, Mus musculus
EVQLQQSGPELKKPGETVKISCKASGYIFTNFGMNWVRQAPGKALKWMGWINTYTGEQIYADDFKGRFAFSLETSASTAYLQINNLKNEDTATYFCARGEIYYGYDVGFVYWGQGTLVTVSA

SEQ ID NO: 80 - DNA, 366 bp, Mus musculus
gaggtccagttgcagcagtctggacctgagctgaagaagcctggagagacagtcaagatctcctgcaaggcttctgggtatatcttcacgaactttggaatgaactgggtgaggcaggctccaggaaaggctttaaagtggatgggctggataaacacctacactggagaacaaatatatgctgatgacttcaagggacggtttgccttctctttggaaacctctgccagcactgcctatttgcagatcaacaacctcaaaaatgaggacacggctacatatttctgtgcaagaggggagatctactatggttacgacgtgggctttgtttactggggccaagggactctggtcactgtctctgca

SEQ ID NO: 81 - protein, 111 aa, Mus musculus
DIVLTQSPASLTVSLGQRATISCGASKSVSTSGYSYMHWYQQKPGQPPKLLIYLGSDLESGVPARFSGSGSGTDFTLNIHPVEEEDAATYYCQHSRELPWTFGGGTKLEIK

SEQ ID NO: 82 - DNA, 333 bp, Mus musculus
gacattgtgctgacacagtctcctgcttccttaactgtatctctggggcagagggccaccatctcatgcggggccagcaaaagtgtcagtacatctggctatagttatatgcactggtaccaacagaaaccaggacagccacccaaactcctcatctatcttggatccgacctagaatctggggtccctgccaggttcagtggcagtgggtctgggacagacttcaccctcaacatccatcctgtggaggaggaggatgctgcaacctattactgtcaacacagtagggaacttccgtggacgttcggtggaggcaccaagctggaaatcaaa

SEQ ID NO: 83 - protein, 15 aa, Mus musculus
GASKSVSTSGYSYMH

SEQ ID NO: 84 - protein, 7 aa, Mus musculus
LGSDLES

SEQ ID NO: 85 - protein, 111 aa, Mus musculus
NIVLTQSPASLAVSLGQRATISCRASESVDSSGNSFMHWYQQKPGQPPKLLIYLESNLESGVPARFSGSGSRTDFTLTIEPVEADDAATYYCQQSNEDPWTFGGGTKLEIK

SEQ ID NO: 86 - DNA, 333 bp, Mus musculus
aacattgtgctgacccaatctccagcttctttggctgtgtctctagggcagagggccaccatatcctgcagagccagtgaaagtgttgatagttctggcaatagttttatgcactggtaccagcagaaaccaggacagccacccaaactcctcatctatcttgaatccaacctagaatctggggtccctgccaggttcagtggcagtgggtctaggacagacttcaccctcaccattgaacctgtggaggctgatgatgctgcaacctattactgtcagcaaagtaatgaggatccgtggacgttcggtggaggcaccaagctggaaatcaaa

SEQ ID NO: 87 - protein, 15 aa, Mus musculus
RASESVDSSGNSFMH

SEQ ID NO: 88 - protein, 7 aa, Mus musculus
LESNLES

SEQ ID NO: 89 - protein, 9 aa, Mus musculus
QQSNEDPWT

SEQ ID NO: 90 - protein, 111 aa, Mus musculus
DIVLTQSPASLAISLGQRATISCRASRSVTTSGYSYIHWYQQKPGQPPKLLIYLASDLEAGVPARFSGSGSGTDFTLNIHPVEEEDAATYYCQHSRELPWTFGGGTKLEIK

SEQ ID NO: 91 - DNA, 333 bp, Mus musculus
gacattgtgctgacacagtctcctgcttccttagctatatctctggggcagagggccaccatctcatgcagggccagcagaagtgtcactacatctggctatagttatatacactggtaccaacagaaaccaggacagccacccaaactcctcatctatcttgcatccgacctagaagctggggtccctgccaggttcagtggcagtgggtctgggacagacttcaccctcaacatccatcctgtggaggaagaggatgctgcaacctattactgtcagcacagtagggagcttccgtggacgttcggtggaggcaccaagctggaaataaaa

SEQ ID NO: 92 - protein, 15 aa, Mus musculus
RASRSVTTSGYSYIH

SEQ ID NO: 93 - protein, 109 aa, Homo sapiens
QAVVTQEPSLTVSPGGTVTLTCRSSTGAVTTSNYANWVQQKPGQAPRGLIGGTNKRAPWTPARFSGSLLGGKAALTLSGAQPEDEAEYYCALWYSNLWVFGGGTKLTVL

SEQ ID NO: 94 - DNA, 327 bp, Homo sapiens
caggctgtggtcacacaagagcctagcctgacagtgtctcctggcggcacagtgaccctgacctgtagatcttctacaggcgccgtgaccaccagcaactacgctaattgggtgcagcagaagcccggccaggctcctagaggactgatcggcggaacaaacaagagagccccttggacacccgccagattctctggatctctgctcggcggaaaggccgctctgacactttctggtgctcagcctgaggacgaggccgagtactattgtgccctgtggtacagcaacctgtgggtgttcggcggaggcaccaaactgacagttctg

SEQ ID NO: 95 - protein, 13 aa, Homo sapiens
SSTGAVTTSNYAN

SEQ ID NO: 96 - protein, 8 aa, Homo sapiens
GGTNKRAP

SEQ ID NO: 97 - protein, 9 aa, Homo sapiens
ALWYSNLWV

SEQ ID NO: 98 - protein, 109 aa, Homo sapiens
QAVVTQEPSLTVSPGGTVTLTCRSSTGAVTTSNYANWVQEKPGQAPRGLIGGTNKRAPWTPARFSGSLLGGKAALTLSGAQPEDEAEYYCALWYSNLWVFGGGTKLTVL

SEQ ID NO: 99 - DNA, 327 bp, Homo sapiens
caggctgtggtcacacaagagcctagcctgacagtgtctcctggcggcacagtgaccctgacctgtagatcttctacaggcgccgtgaccaccagcaactacgctaattgggtgcaggagaagcccggccaggctcctagaggactgatcggcggaacaaacaagagagccccttggacacccgccagattctctggatctctgctcggcggaaaggccgctctgacactttctggtgctcagcctgaggacgaggccgagtactattgtgccctgtggtacagcaacctgtgggtgttcggcggaggcaccaaactgacagttctg

SEQ ID NO: 100 - protein, 109 aa, Homo sapiens
EAVVTQEPSLTVSPGGTVTLTCESSDGAVTTSNYANWVQEKPGQAPRGLIGGTNKEAPWTPARFSGSLLGGKAALTLSGAQPEDEAEYYCALWYSNLWVFGGGTKLTVL

SEQ ID NO: 101 - DNA, 327 bp, Homo sapiens
gaggctgtggtcacacaagagcctagcctgacagtgtctcctggcggcacagtgaccctgacctgtgagtcttctgacggcgccgtgaccaccagcaactacgctaattgggtgcaggagaagcccggccaggctcctagaggactgatcggcggaacaaacaaggaggccccttggacacccgccagattctctggatctctgctcggcggaaaggccgctctgacactttctggtgctcagcctgaggacgaggccgagtactattgtgccctgtggtacagcaacctgtgggtgttcggcggaggcaccaaactgacagttctg

SEQ ID NO: 102 - protein, 13 aa, Homo sapiens
SSDGAVTTSNYAN

SEQ ID NO: 103 - protein, 8 aa, Homo sapiens
GGTNKEAP

SEQ ID NO: 104 - protein, 109 aa, Homo sapiens
QAVVTQEPSLTVSPGGTVTLTCESSDGAVTTSNYANWVQEKPGQAPRGLIGGTNKEAPWTPARFSGSLLGGKAALTLSGAQPEDEAEYYCALWYSNLWVFGGGTKLTVL

SEQ ID NO: 105 - DNA, 327 bp, Homo sapiens
caggctgtggtcacacaagagcctagcctgacagtgtctcctggcggcacagtgaccctgacctgtgagtcttctgacggcgccgtgaccaccagcaactacgctaattgggtgcaggagaagcccggccaggctcctagaggactgatcggcggaacaaacaaggaggccccttggacacccgccagattctctggatctctgctcggcggaaaggccgctctgacactttctggtgctcagcctgaggacgaggccgagtactattgtgccctgtggtacagcaacctgtgggtgttcggcggaggcaccaaactgacagttctg

SEQ ID NO: 106 - protein, 109 aa, Homo sapiens
QAVVTQEPSLTVSPGGTVTLTCRSSTGAVTTSNYANWVQEKPGQAPRGLIGGTNKRAPWTPARFSGSLLGGKAALTITGAQAEDEAEYYCVLWYSNLWVFGGGTKLTVL

SEQ ID NO: 107 - DNA, 327 bp, Homo sapiens
caggctgtggtcacacaagagcctagcctgacagtgtctcctggcggcacagtgaccctgacctgtagatcttctacaggcgccgtgaccaccagcaactacgctaattgggtgcaggagaagcccggccaggctcctagaggactgatcggcggaacaaacaagagagccccttggacacccgccagattctctggatctctgctcggcggaaaggccgctctgacaatcactggtgctcaggctgaggacgaggccgagtactattgtgtgctgtggtacagcaacctgtgggtgttcggcggaggcaccaaactgacagttctg

SEQ ID NO: 108 - protein, 9 aa, Homo sapiens
VLWYSNLWV

SEQ ID NO: 109 - protein, 109 aa, Homo sapiens
QAVVTQEPSLTVSPGGTVTLTCRSSTGAVTTSNYANWFQEKPGQAPRGLIYGTNKRAPWTPARFSGSLLGGKAALTLSGAQAEDEAEYYCVLWYSNLWVFGGGTKLTVL

SEQ ID NO: 110 - DNA, 327 bp, Homo sapiens
caggctgtggtcacacaagagcctagcctgacagtgtctcctggcggcacagtgaccctgacctgtagatcttctacaggcgccgtgaccaccagcaactacgctaattggttccaggagaagcccggccaggctcctagaggactgatctacggaacaaacaagagagccccttggacacccgccagattctctggatctctgctcggcggaaaggccgctctgacactttctggtgctcaggctgaggacgaggccgagtactattgtgtcctgtggtacagcaacctgtgggtgttcggcggaggcaccaaactgacagttctg

SEQ ID NO: 111 - protein, 8 aa, Homo sapiens
YGTNKRAP

SEQ ID NO: 112 - protein, 125 aa, Homo sapiens
EVQLVESGGGLVQPGGSLRLSCAASGFTFNTYAMNWVRQAPGKGLEWVSRIRSKYNNYATYYADSVKDRFTISRDDSKNTLYLQMNSLKTEDTAVYYCVRHGNFGNSYVSWFAYWGQGTLVTVSS

SEQ ID NO: 113 - DNA, 375 bp, Homo sapiens
gaggtgcagctggttgaatctggcggaggactggttcagcctggcggatctctgagactgtcttgtgccgccagcggcttcaccttcaacacctacgctatgaactgggtccgacaggcccctggcaaaggactggaatgggtgtccagaatcaggtccaagtacaacaactacgccacctactacgccgacagcgtgaaggacagattcaccatcagcagggacgacagcaagaacaccctgtacctgcagatgaacagcctgagagccgaggacaccgccgtgtactactgtgtcagacacggcaacttcggcaacagctatgtgtcttggtttgcctactggggccagggcacactggtcacagttagctct

SEQ ID NO: 114 - protein, 6 aa, Homo sapiens
NTYAMN

SEQ ID NO: 115 - protein, 18 aa, Homo sapiens
IRSKYNNYATYYADSVKD

SEQ ID NO: 116 - protein, 14 aa, Homo sapiens
HGNFGNSYVSWFAY

SEQ ID NO: 117 - protein, 125 aa, Homo sapiens
EVQLVESGGGLVQPGGSLRLSCAASGFTFSTYAMNWVRKAPGKGLEWVSRIRSKYNNYATYYADSVKDRFTISRDDSKNTLYLQMNSLRAEDTAVYYCVRHGNFGNSYVSWFAYWGQGTLVTVSS

SEQ ID NO: 118 - DNA, 375 bp, Homo sapiens
gaggtgcagctggttgaatctggcggaggactggttcagcctggcggatctctgagactgtcttgtgccgccagcggcttcaccttctccacctacgctatgaactgggtccgaaaggcccctggcaaaggactggaatgggtgtccagaatcaggtccaagtacaacaactacgccacctactacgccgacagcgtgaaggacagattcaccatcagcagggacgacagcaagaacaccctgtacctgcagatgaacagcctgagagccgaggacaccgccgtgtactactgtgtcagacacggcaacttcggcaacagctatgtgtcttggtttgcctactggggccagggcacactggtcacagttagctct

SEQ ID NO: 119 - protein, 6 aa, Homo sapiens
STYAMN

SEQ ID NO: 120 - protein, 125 aa, Homo sapiens
EVQLVESGGGLVQPGGSLRLSCAASGFTFSTYAMNWVRKAPGKGLEWVSRIRSKYNNYATYYADSVKDRFTISRDDSKNTLYLQMNSLRAEDTAVYYCVRHGNFGESYVSWFAYWGQGTLVTVSS

SEQ ID NO: 121 - DNA, 375 bp, Homo sapiens
gaggtgcagctggttgaatctggcggaggactggttcagcctggcggatctctgagactgtcttgtgccgccagcggcttcaccttctccacctacgctatgaactgggtccgaaaggcccctggcaaaggactggaatgggtgtccagaatcaggtccaagtacaacaactacgccacctactacgccgacagcgtgaaggacagattcaccatcagcagggacgacagcaagaacaccctgtacctgcagatgaacagcctgagagccgaggacaccgccgtgtactactgtgtcagacacggcaacttcggcgagagctatgtgtcttggtttgcctactggggccagggcacactggtcacagttagctct

SEQ ID NO: 122 - protein, 14 aa, Homo sapiens
HGNFGESYVSWFAY

SEQ ID NO: 123 - protein, 125 aa, Homo sapiens
EVQLVESGGGLVQPGGSLRLSCAASGFTFSTYAMNWVRKAPGKGLEWVSRIRSKYNNYATYYADSVKDRFTISRDDSKNTLYLQMNSLRAEDTAVYYCVRHGNFGQSYVSWFAYWGQGTLVTVSS

SEQ ID NO: 124 - DNA, 375 bp, Homo sapiens
gaggtgcagctggttgaatctggcggaggactggttcagcctggcggatctctgagactgtcttgtgccgccagcggcttcaccttctccacctacgctatgaactgggtccgaaaggcccctggcaaaggactggaatgggtgtccagaatcaggtccaagtacaacaactacgccacctactacgccgacagcgtgaaggacagattcaccatcagcagggacgacagcaagaacaccctgtacctgcagatgaacagcctgagagccgaggacaccgccgtgtactactgtgtcagacacggcaacttcggccagagctatgtgtcttggtttgcctactggggccagggcacactggtcacagttagctct

SEQ ID NO: 125 - protein, 14 aa, Homo sapiens
HGNFGQSYVSWFAY

SEQ ID NO: 126 - protein, 125 aa, Homo sapiens
EVQLVESGGGLVQPGGSLRLSCAASGFTFSTYAMNWVRKAPGKGLEWVSRIRSKYNNYATYYADSVKDRFTISRDDSKNTLYLQMNSLRAEDTAVYYCVRHGNFGDSYVSWFAYWGQGTLVTVSS

SEQ ID NO: 127 - DNA, 375 bp, Homo sapiens
gaggtgcagctggttgaatctggcggaggactggttcagcctggcggatctctgagactgtcttgtgccgccagcggcttcaccttctccacctacgctatgaactgggtccgaaaggcccctggcaaaggactggaatgggtgtccagaatcaggtccaagtacaacaactacgccacctactacgccgacagcgtgaaggacagattcaccatcagcagggacgacagcaagaacaccctgtacctgcagatgaacagcctgagagccgaggacaccgccgtgtactactgtgtcagacacggcaacttcggcgacagctatgtgtcttggtttgcctactggggccagggcacactggtcacagttagctct

SEQ ID NO: 128 - protein, 14 aa, Homo sapiens
HGNFGDSYVSWFAY

SEQ ID NO: 129 - protein, 125 aa, Homo sapiens
EVQLVESGGGLVQPGGSLRLSCAASGFTFSTYAMNWVRKAPGKGLEWVSRIRSKYNNYATYYADSVKDRFTISRDDSKNTLYLQMNSLRAEDTAVYYCVRHGNFGTSYVSWFAYWGQGTLVTVSS

SEQ ID NO: 130 - DNA, 375 bp, Homo sapiens
gaggtgcagctggttgaatctggcggaggactggttcagcctggcggatctctgagactgtcttgtgccgccagcggcttcaccttctccacctacgctatgaactgggtccgaaaggcccctggcaaaggactggaatgggtgtccagaatcaggtccaagtacaacaactacgccacctactacgccgacagcgtgaaggacagattcaccatcagcagggacgacagcaagaacaccctgtacctgcagatgaacagcctgagagccgaggacaccgccgtgtactactgtgtcagacacggcaacttcggcaccagctatgtgtcttggtttgcctactggggccagggcacactggtcacagttagctct

SEQ ID NO: 131 - protein, 14 aa, Homo sapiens
HGNFGTSYVSWFAY

SEQ ID NO: 132 - protein, 125 aa, Homo sapiens
EVQLVESGGGLVQPGGSLRLSCAASGFTFSDYAMNWVRKAPGKGLEWVSRIRSKYNNYATYYADSVEDRFTISRDDSKNTLYLQMNSLRAEDTAVYYCVRHGNFGNSYVSWFAYWGQGTLVTVSS

SEQ ID NO: 133 - DNA, 375 bp, Homo sapiens
gaggtgcagctggttgaatctggcggaggactggttcagcctggcggatctctgagactgtcttgtgccgccagcggcttcaccttctccgactacgctatgaactgggtccgaaaggcccctggcaaaggactggaatgggtgtccagaatcaggtccaagtacaacaactacgccacctactacgccgacagcgtggaggacagattcaccatcagcagggacgacagcaagaacaccctgtacctgcagatgaacagcctgagagccgaggacaccgccgtgtactactgtgtcagacacggcaacttcggcaacagctatgtgtcttggtttgcctactggggccagggcacactggtcacagttagctct

SEQ ID NO: 134 - protein, 6 aa, Homo sapiens
SDYAMN

SEQ ID NO: 135 - protein, 18 aa, Homo sapiens
IRSKYNNYATYYADSVED

SEQ ID NO: 136 - protein, 125 aa, Homo sapiens
EVQLVESGGGLVQPGGSLRLSCAASGFTFNTYAMNWVRKAPGKGLEWVGRIRSKYNNYATYYADSVKDRFTISRDDSKNSLYLQMNSLKTEDTAVYYCARHGNFGNSYVSWFAYWGQGTLVTVSS

SEQ ID NO: 137 - DNA, 375 bp, Homo sapiens
gaggtgcagctggttgaatctggcggaggactggttcagcctggcggatctctgagactgtcttgtgccgccagcggcttcaccttcaacacctacgctatgaactgggtccgaaaggcccctggcaaaggactggaatgggtgggaagaatcaggtccaagtacaacaactacgccacctactacgccgacagcgtgaaggacagattcaccatcagcagggacgacagcaagaacagcctgtacctgcagatgaacagcctgaaaaccgaggacaccgccgtgtactactgtgccagacacggcaacttcggcaacagctatgtgtcttggtttgcctactggggccagggcacactggtcacagttagctct

SEQ ID NO: 138 - protein, 125 aa, Homo sapiens
EVQLVESGGGLVQPGGSLRLSCAASGFTFNTYAMNWVRKAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 139 - DNA, 375 bp, Homo sapiens
gaggtgcagctggttgaatctggcggaggactggttcagcctggcggatctctgagactgtcttgtgccgccagcggcttcaccttcaacacctacgctatgaactgggtccgaaaggcccctggcaaaggactggaatgggtgggaagaatcaggtccaagtacaacaactacgccacctactacgccgacagcgtgaaggacagattcaccatcagcagggacgacagcaagaacagcctgtacctgcagatgaacagcctgaaaaccgaggacaccgccgtgtactactgtgtcagacacggcaacttcggcaacagctatgtgtcttggtttgcctactggggccagggcacactggtcacagttagctct

SEQ ID NO: 140 - protein, 125 aa, Homo sapiens
EVQLVESGGGLVQPGGSLRLSCAASGFTFNTYAMNWVRKAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 141 - DNA, 375 bp, Homo sapiens
gaggtgcagctggttgaatctggcggaggactggttcagcctggcggatctctgagactgtcttgtgccgccagcggcttcaccttcaacacctacgctatgaactgggtccgaaaggcccctggcaaaggactggaatgggtggccagaatcaggtccaagtacaacaactacgccacctactacgccgacagcgtgaaggacagattcaccatcagcagggacgacagcaagaacagcctgtacctgcagatgaacagcctgaaaaccgaggacaccgccgtgtactactgtgtcagacacggcaacttcggcaacagctatgtgtcttggtttgcctactggggccagggcacactggtcacagttagctct

SEQ ID NO: 142 - protein, 107 aa, Homo sapiens
DIQMTQSPSSLSASVGDRVTITCQASQDVDTAVAWYQKKPGKAPKLLIYWTSTRHTGVPSRFSGSGSGTDFTFTISSLQPEDIATYFCQQFSSYPLTFGQGTKVEIK

SEQ ID NO: 143 - DNA, 321 bp, Homo sapiens
gacatccagatgacacagagccctagcagcctgtctgccagcgtgggagacagagtgaccatcacctgtcaggccagccaggatgtggatacagccgtggcctggtatcagaagaagcctggcaaggcccctaagctgctgatctactggaccagcaccagacacacaggcgtgccctctagattcagcggctctggctctggcaccgacttcaccttcacaatcagcagcctgcagcctgaggatatcgctacctacttctgccagcagttcagcagctaccctctgacattcggccagggcaccaaggtggaaatcaag

SEQ ID NO: 144 - protein, 11 aa, Homo sapiens
QASQDVDTAVA

SEQ ID NO: 145 - protein, 121 aa, Homo sapiens
QVQLVQSGAEVKKPGSSVKVSCKASGYTFTNYWIGWVREAPGQGLEWMGDIYPGSGDTNYNGKFQGRVTLTADISSNTAYMELSSLRSEDTAVYYCARSYYRNDAASFAYWGQGTLVTVSS

SEQ ID NO: 146 - DNA, 363 bp, Homo sapiens
caggttcagctggttcagtctggcgccgaagtgaagaaacctggcagcagcgtgaaggtgtcctgcaaggctagcggctacacattcaccaactactggatcggctgggtccgagaggctcctggacaaggacttgagtggatgggcgacatctaccctggcagcggcgacacaaactacaacggcaagttccagggcagagtgaccctgacagccgacatcagcagcaacaccgcctacatggaactgagcagcctgagaagcgaggacaccgccgtgtactactgcgccagaagctactacagaaacgacgccgccagcttcgcttactggggacagggaacactggtcaccgtgtctagt

SEQ ID NO: 147 - protein, 17 aa, Homo sapiens
DIYPGSGDTNYNGKFQG

SEQ ID NO: 148 - protein, 111 aa, Homo sapiens
DIVLTQSPASLAVSPGQRATITCRASKSVSTSGYSYMHWYQKKPGQPPKLLIYLASNLESGVPARFSGSGSGTDFTLTINPVEANDTANYYCQHSRELPWTFGQGTKVEIK

SEQ ID NO: 149 - DNA, 333 bp, Homo sapiens
gacatcgtgctgacacagagccctgcttctctggctgtgtctcctggccagagagccaccatcacctgtagagccagcaagagcgtgtccaccagcggctactcttacatgcactggtatcagaagaagcccggccagcctcctaagctgctgatctacctggctagcaacctcgaaagcggagtgcctgctagattttctggcagcggctctggcaccgacttcaccctgacaatcaaccccgtggaagccaacgacaccgccaactactactgccagcacagcagagagctgccctggacatttggccagggcaccaaggtggaaatcaag

SEQ ID NO: 150 - protein, 122 aa, Homo sapiens
QVQLVQSGSELKKPGASVKVSCKASGYTFTNFGMNWVREAPGQGLEWMGWINTYTGDQIYADDFKGRFAFSLDTSASTAYLQISSLKAEDTAVYFCARGEIYYGYDVGFVYWGQGTLVTVSS

SEQ ID NO: 151 - DNA, 366 bp, Homo sapiens
caggttcagctggtgcagtctggcagcgagctgaagaaacctggcgcctctgtgaaggtgtcctgcaaggctagcggctacacattcaccaacttcggcatgaactgggtccgagaggctcctggacagggactcgaatggatgggctggatcaacacctacaccggcgaccagatctacgccgacgacttcaagggcagattcgccttcagcctggacacctctgccagcacagcttacctgcagatcagcagcctgaaggccgaggataccgccgtgtacttctgtgccagaggcgagatctactacggctacgatgtgggcttcgtctactggggacagggaacactggtcaccgttagctct

SEQ ID NO: 152 - protein, 111 aa, Homo sapiens
DIVLTQSPASLAVSPGQRATITCRASRSVTTSGYSYIHWYQKKPGQPPKLLIYLASDLEAGVPARFSGSGSGTDFTLTINPVEANDTANYYCQHSRELPWTFGQGTKVEIK

SEQ ID NO: 153 - DNA, 333 bp, Homo sapiens
gacatcgtgctgacacagagccctgcttctctggctgtgtctcctggccagagagccaccatcacctgtagagccagcagaagcgtgaccaccagcggctactcttacatccactggtatcagaagaagcccggccagcctcctaagctgctgatctacctggccagcgatctggaagctggcgtgccagctagattttctggcagcggctctggcaccgacttcaccctgacaatcaaccccgtggaagccaacgacaccgccaactactactgccagcacagcagagagctgccctggacatttggccagggcaccaaggtggaaatcaag

SEQ ID NO: 154 - protein, 122 aa, Homo sapiens
QVQLVQSGSELKKPGASVKVSCKASGYIFTNFGMNWVREAPGQGLEWMGWINTYTGEQIYADDFKGRFAFSLDTSASTAYLQISSLKAEDTAVYFCARGEIYYGYDVGFVYWGQGTLVTVSS

SEQ ID NO: 155 - DNA, 366 bp, Homo sapiens
caggttcagctggtgcagtctggcagcgagctgaagaaacctggcgcctctgtgaaggtgtcctgcaaggctagcggctacatcttcaccaacttcggcatgaactgggtccgagaggctcctggacagggactcgaatggatgggctggatcaacacctacaccggcgagcagatctacgccgatgacttcaaaggcagattcgcgttcagcctggacaccagcgccagcacagcttacctgcagatcagctctctgaaggccgaggataccgccgtgtacttctgtgccagaggcgagatctactacggctacgacgtgggctttgtgtactggggccagggaacactggtcaccgttagctct

SEQ ID NO: 156 - protein, 111 aa, Homo sapiens
DIVLTQSPASLAVSPGQRATITCRASKSVSTSGYSYMHWYQKKPGQPPKLLIYLGSDLESGVPARFSGSGSGTDFTLTINPVEANDTANYYCQHSRELPWTFGQGTKVEIK

SEQ ID NO: 157 - DNA, 333 bp, Homo sapiens
gacatcgtgctgacacagagccctgcttctctggctgtgtctcctggccagagagccaccatcacctgtagagccagcaagagcgtgtccaccagcggctactcttacatgcactggtatcagaagaagcccggccagcctcctaagctgctgatctacctgggtagcgacctcgaaagcggagtgcctgctagattttctggcagcggctctggcaccgacttcaccctgacaatcaaccccgtggaagccaacgacaccgccaactactactgccagcacagcagagagctgccctggacatttggccagggcaccaaggtggaaatcaag

SEQ ID NO: 158 - protein, 122 aa, Homo sapiens; identical to SEQ ID NO: 154

SEQ ID NO: 159 - DNA, 366 bp, Homo sapiens; identical to SEQ ID NO: 155

SEQ ID NO: 160 - protein, 111 aa, Homo sapiens; identical to SEQ ID NO: 148

SEQ ID NO: 161 - DNA, 333 bp, Homo sapiens; identical to SEQ ID NO: 149

SEQ ID NO: 162 - protein, 122 aa, Homo sapiens; identical to SEQ ID NO: 154

SEQ ID NO: 163 - DNA, 366 bp, Homo sapiens; identical to SEQ ID NO: 155

SEQ ID NO: 164 - protein, 111 aa, Homo sapiens; identical to SEQ ID NO: 148

SEQ ID NO: 165 - DNA, 333 bp, Homo sapiens; identical to SEQ ID NO: 149

SEQ ID NO: 166 - protein, 122 aa, Homo sapiens
QVQLVQSGSELKKPGASVKVSCKASGYTFTNFGMNWVREAPGQGLEWMGWINTYTGDLIYGDDFKGRFAFSLDTSASTAYLQISSLKAEDTAVYFCARGEIYYGYDVGFAYWGQGTLVTVSS

SEQ ID NO: 167 - DNA, 366 bp, Homo sapiens
caggttcagctggtgcagtctggcagcgagctgaagaaacctggcgcctctgtgaaggtgtcctgcaaggctagcggctacacattcaccaacttcggcatgaactgggtccgagaggctcctggacagggactcgaatggatgggctggatcaacacctacaccggcgacctgatctacggcgacgacttcaagggcagattcgccttcagcctggacacctctgccagcacagcttacctgcagatcagcagcctgaaggccgaggataccgccgtgtacttctgtgccagaggcgagatctactacggctacgatgtgggcttcgcctactggggacagggaacactggtcaccgttagctct

SEQ ID NO: 168 - protein, 111 aa, Homo sapiens; identical to SEQ ID NO: 148

SEQ ID NO: 169 - DNA, 333 bp, Homo sapiens; identical to SEQ ID NO: 149

SEQ ID NO: 170 - protein, 122 aa, Homo sapiens
QVQLVQSGSELKKPGASVKVSCKASGYTFTNYGMNWVREAPGQGLEWMGWINTYTGERIYADDFKGRFAFSLDTSASTAYLQISSLKAEDTAVYFCARGEIYYGYDVGFVYWGQGTLVTVSS

SEQ ID NO: 171 - DNA, 366 bp, Homo sapiens
caggttcagctggtgcagtctggcagcgagctgaagaaacctggcgcctctgtgaaggtgtcctgcaaggctagcggctacacattcaccaactacggcatgaactgggtccgagaggctcctggacagggactcgaatggatgggctggatcaacacctacaccggcgaaaggatctacgccgacgacttcaagggcagattcgccttcagcctggacacctctgccagcacagcttacctgcagatcagcagcctgaaggccgaggataccgccgtgtacttctgtgccagaggcgagatctactacggctacgatgtgggcttcgtctactggggacagggaacactggtcaccgttagctct

SEQ ID NO: 172 - protein, 218 aa, Homo sapiens
DIVLTQSPASLAVSPGQRATITCRASKSVSTSGYSYMHWYQKKPGQPPKLLIYLASNLESGVPARFSGSGSGTDFTLTINPVEAEDTANYYCQHSRELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - DNA, 654 bp, Homo sapiens
gacatcgtgctgacacagagccctgcttctctggctgtgtctcctggccagagagccaccatcacctgtagagccagcaagagcgtgtccaccagcggctactcttacatgcactggtatcagaagaagcccggccagcctcctaagctgctgatctacctggctagcaacctcgaaagcggagtgcctgctagattttctggcagcggctctggcaccgacttcaccctgacaatcaaccccgtggaagccgaagacaccgccaactactactgccagcacagcagagagctgccctggacatttggccagggcaccaaggtggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 174 - protein, 449 aa, Homo sapiens
QVQLVQSGSELKKPGASVKVSCKASGYIFTNFGMNWVREAPGQGLEWMGWINTYTGEQIYADGFTGRFVFSLDTSASTAYLQISSLKAEDTAVYFCARGEIYYGYDVGFVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLASSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLGK

SEQ ID NO: 175 - DNA, 1347 bp, Homo sapiens
caggttcagctggtgcagtctggcagcgagctgaagaaacctggcgcctctgtgaaggtgtcctgcaaggctagcggctacatcttcaccaacttcggcatgaactgggtccgagaggctcctggacagggactcgaatggatgggctggatcaacacctacaccggcgagcagatctacgccgatggcttcacaggcagattcgtgttcagcctggacaccagcgccagcacagcttacctgcagatcagctctctgaaggccgaggataccgccgtgtacttctgtgccagaggcgagatctactacggctacgacgtgggctttgtgtactggggccagggaacactggtcaccgttagctctgctagcaccaagggccccagcgtgttccccctggccccttgcagcagaagcaccagcgagagcacagccgccctgggctgcctggtgaaggactacttccccgagcccgtgaccgtgtcctggaacagcggcgctctgaccagcggcgtgcataccttccccgccgtgctccagagcagcggactgtactccctgagcagcgtggtgaccgtgccttccagcagcctgggcaccaagacctacacctgcaacgtggaccacaagcccagcaacaccaaggtggacaagagagtggagagcaagtacggccctccctgccccccttgccctgcccccgagttcgagggcggacctagcgtgttcctgttcccccccaagcccaaggacaccctgatgatcagcagaacccccgaggtgacctgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctggccagcagcatcgagaagaccatcagcaaggccaagggccagccacgggagccccaggtctgcaccctgccacctagccaagaggagatgaccaagaaccaggtgtccctgagctgtgccgtgaaaggcttctatcccagcgatatcgccgtggagtgggagagcaacggccagcccgagaacaactacaagaccaccccccctgtgctggacagcgacggcagcttcttcctggtttccaagctgaccgtggacaagtccagatggcaggagggcaacgtcttcagctgctccgtgatgcacgaggccctgcacaaccactacacccagaagtccctgagcctgagcctgggcaag

SEQ ID NO: 176 - protein, 218 aa, Homo sapiens
DIVLTQSPASLAVSPGQRATITCRASKSVTTSGYSYIHWYQKKPGQPPKLLIYLASDLEAGVPARFSGSGSGTDFTLTINPVEAEDTANYYCQHSRELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - DNA, 654 bp, Homo sapiens
gacatcgtgctgacacagagccctgcttctctggctgtgtctcctggccagagagccaccatcacctgtagagccagcaagagcgtgaccaccagcggctactcttacatccactggtatcagaagaagcccggccagcctcctaagctgctgatctacctggccagcgatctggaagctggcgtgccagctagattttctggcagcggctctggcaccgacttcaccctgacaatcaaccccgtggaagccgaagacaccgccaactactactgccagcacagcagagagctgccctggacatttggccagggcaccaaggtggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 178 - protein, 449 aa, Homo sapiens
QVQLVQSGSELKKPGASVKVSCKASGYIFTNFGMNWVREAPGQGLEWMGWINTYTGEQIYADGFTGRFVFSLDTSVSTAYLQISSLKAEDTAVYFCARGEIYYGYDVGFVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLASSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLGK

SEQ ID NO: 179 - DNA, 1347 bp, Homo sapiens
caggttcagctggtgcagtctggcagcgagctgaagaaacctggcgcctctgtgaaggtgtcctgcaaggctagcggctacatcttcaccaacttcggcatgaactgggtccgagaggctcctggacagggactcgaatggatgggctggatcaacacctacaccggcgagcagatctacgccgatggcttcacaggcagattcgtgttcagcctggacaccagcgtcagcacagcttacctgcagatcagctctctgaaggccgaggataccgccgtgtacttctgtgccagaggcgagatctactacggctacgacgtgggctttgtgtactggggccagggaacactggtcaccgttagctctgctagcaccaagggccccagcgtgttccccctggccccttgcagcagaagcaccagcgagagcacagccgccctgggctgcctggtgaaggactacttccccgagcccgtgaccgtgtcctggaacagcggcgctctgaccagcggcgtgcataccttccccgccgtgctccagagcagcggactgtactccctgagcagcgtggtgaccgtgccttccagcagcctgggcaccaagacctacacctgcaacgtggaccacaagcccagcaacaccaaggtggacaagagagtggagagcaagtacggccctccctgccccccttgccctgcccccgagttcgagggcggacctagcgtgttcctgttcccccccaagcccaaggacaccctgatgatcagcagaacccccgaggtgacctgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctggccagcagcatcgagaagaccatcagcaaggccaagggccagccacgggagccccaggtctgcaccctgccacctagccaagaggagatgaccaagaaccaggtgtccctgagctgtgccgtgaaaggcttctatcccagcgatatcgccgtggagtgggagagcaacggccagcccgagaacaactacaagaccaccccccctgtgctggacagcgacggcagcttcttcctggtttccaagctgaccgtggacaagtccagatggcaggagggcaacgtcttcagctgctccgtgatgcacgaggccctgcacaaccactacacccagaagtccctgagcctgagcctgggcaag

SEQ ID NO: 180 - protein, 215 aa, Homo sapiens
QAVVTQEPSLTVSPGGTVTLTCRSSTGAVTTSNYANWVQEKPGQAPRGLIGGTNKRAPWTPARFSGSLLGGKAALTITGAQAEDEAEYYCVLWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 181 - DNA, 645 bp, Homo sapiens
caggctgtggtcacacaagagcctagcctgacagtgtctcctggcggcacagtgaccctgacctgtagatcttctacaggcgccgtgaccaccagcaactacgctaattgggtgcaggagaagcccggccaggctcctagaggactgatcggcggaacaaacaagagagccccttggacacccgccagattctctggatctctgctcggcggaaaggccgctctgacaatcactggtgctcaggctgaggacgaggccgagtactattgtgtgctgtggtacagcaacctgtgggtgttcggcggaggcaccaaactgacagttctgggtcagcccaaggcggcgccctcggtcactctgttcccgccctcctctgaggagcttcaagccaacaaggccacactggtgtgtctcataagtgacttctatccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 182 - protein, 452 aa, Homo sapiens
EVQLVESGGGLVQPGGSLRLSCAASGFTFNTYAMNWVRKAPGKGLEWVGRIRSKYNNYATYYADSVKDRFTISRDDSKNSLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLASSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGK

SEQ ID NO: 183 - DNA, 1356 bp, Homo sapiens
gaggtgcagctggttgaatctggcggaggactggttcagcctggcggatctctgagactgtcttgtgccgccagcggcttcaccttcaacacctacgctatgaactgggtccgaaaggcccctggcaaaggactggaatgggtgggaagaatcaggtccaagtacaacaactacgccacctactacgccgacagcgtgaaggacagattcaccatcagcagggacgacagcaagaacagcctgtacctgcagatgaacagcctgaaaaccgaggacaccgccgtgtactactgtgtcagacacggcaacttcggcaacagctatgtgtcttggtttgcctactggggccagggcacactggtcacagttagctctgctagcaccaagggccccagcgtgttccccctggccccttgcagcagaagcaccagcgagagcacagccgccctgggctgcctggtgaaggactacttccccgagcccgtgaccgtgtcctggaacagcggcgctctgaccagcggcgtgcataccttccccgccgtgctccagagcagcggactgtactccctgagcagcgtggtgaccgtgccttccagcagcctgggcaccaagacctacacctgcaacgtggaccacaagcccagcaacaccaaggtggacaagagagtggagagcaagtacggccctccctgccccccttgccctgcccccgagttcgagggcggacctagcgtgttcctgttcccccccaagcccaaggacaccctgatgatcagcagaacccccgaggtgacctgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctggccagcagcatcgagaagaccatcagcaaggccaagggccagccacgggagccccaggtctacaccctgccaccttgtcaagaggagatgaccaagaaccaggtgtccctgtggtgtctggtgaaaggcttctatcccagcgatatcgccgtggagtgggagagcaacggccagcccgagaacaactacaagaccaccccccctgtgctggacagcgacggcagcttcttcctgtactccaagctgaccgtggacaagtccagatggcaggagggcaacgtcttcagctgctccgtgatgcacgaggccctgcacaaccactacacccagaagtccctgagcctgagcctgggcaag

SEQ ID NO: 184 - protein, 109 aa, Mus musculus
QAVVTQESALTTSPGETVTLTCRSSTGAVTTSNYANWVQQKPDHLFTGLIGGTNKRAPGVPARFSGSLIGDKAALTITGAQTEDEAIYFCALWYSNLWVFGGGTKLTVL

SEQ ID NO: 185 - protein, 125 aa, Mus musculus
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS